(19) United States
(12) Patent Application Publication
(10) Pub. No.: US 2014/0249024 A1
(43) Pub. Date: Sep. 4, 2014

(54) Pesticidal Methods Using Substituted 3-pyridyl Thiazole Compounds and Derivatives for Combating Animal Pests II

(71) Applicants: Florian Kaiser, Mannheim (DE); Gemma Veitch, Basel (CH); Arun Narine, Mannheim (DE); Joachim Dickhaut, Heidelberg (DE); Karsten Körber, Eppelheim (DE); Prashant Deshmukh, Mannheim (DE); Nina Gertrud Bandur, Mannheim (DE)

(72) Inventors: Florian Kaiser, Mannheim (DE); Gemma Veitch, Basel (CH); Arun Narine, Mannheim (DE); Joachim Dickhaut, Heidelberg (DE); Karsten Körber, Eppelheim (DE); Prashant Deshmukh, Mannheim (DE); Nina Gertrud Bandur, Mannheim (DE)

(73) Assignee: BASF SE, Ludwigshafen (DE)

(21) Appl. No.: 14/232,428

(22) PCT Filed: Jul. 13, 2012
(86) PCT No.: PCT/EP2012/063819

§ 371 Date: Apr. 11, 2014

Related U.S. Application Data
(60) Provisional application No. 61/508,090, filed on Jul. 15, 2011.

(51) Int. Cl. A01N 43/78; C07F 5/02; C07D 417/04; C07D 417/14

(52) U.S. Cl. CPC A01N 43/78; C07D 417/14; C07F 5/025; C07D 417/04
USPC 504/100; 514/342; 546/270.4; 546/13; 514/275; 544/331; 514/256; 544/333; 514/333; 546/256; 544/296

(57) ABSTRACT

The present invention relates to pesticidal methods for the use and application of substituted 3-pyridyl thiazole compounds and the stereoisomers, salts, tautomers and N-oxides thereof and to compositions comprising the same. The invention also relates to insecticidal substituted 3-pyridyl thiazole compounds or of the compositions comprising such compounds for combating invertebrate pests and uses thereof.

The substituted 3-pyridyl thiazole compounds of the present invention are defined by the following general formula (I):

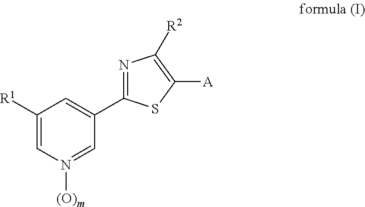

wherein R¹, R² A and m are defined as in the description.

The present invention relates to pesticidal methods for the use and application of substituted 3-pyridyl thiazole compounds and the stereoisomers, salts, tautomers and N-oxides thereof and to compositions comprising the same. The invention also relates to insecticidal substituted 3-pyridyl thiazole compounds or of the compositions comprising such compounds for combating invertebrate pests and uses thereof.

Invertebrate pests and in particular insects, arthropods and nematodes destroy growing and harvested crops and attack wooden dwelling and commercial structures, thereby causing large economic loss to the food supply and to property. While a large number of pesticidal agents are known, due to the ability of target pests to develop resistance to said agents, there is an ongoing need for new agents for combating invertebrate pests such as insects, arachnids and nematodes. It is therefore an object of the present invention to provide compounds having a good pesticidal activity and showing a broad activity spectrum against a large number of different invertebrate pests, especially against difficult to control insects, arachnids and nematodes.

It has been found that these objectives can be achieved by substituted 3-pyridyl thiazole compounds of the general formula (I), as defined below, including their stereoisomers, their salts, in particular their agriculturally or veterinarily acceptable salts, their tautomers and their N-oxides.

Therefore, in a first aspect the present invention relates to methods for using substituted 3-pyridyl thiazole compounds of formula (I):

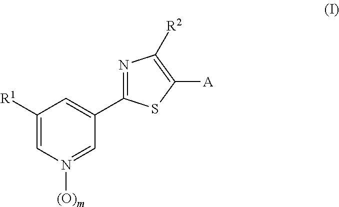

wherein
m is 0 or 1;
R¹ is selected from the group consisting of hydrogen, cyano or halogen;
R² is selected from the group consisting of halogen or C₁-C₆-haloalkyl, the latter may be partially or fully halogenated and may optionally be further substituted by 1, 2, 3 or 4 radicals R⁷;
A is a molecular group representing a nitrogen containing ring system selected from
   a 4-, 5-, 6- or 7-membered heterocyclic or heteroaromatic ring system, or
   a 8-10-membered heteroaromatic bicyclic ring system, wherein each ring is on its carbon atoms optionally substituted with 1, 2, 3, 4, 5 or 6 substituents R⁶, depending on the number of R⁶ present on the ring system and depending of the nature of R², wherein each
   R⁶, when R² is C₁-C₆-haloalkyl and the number of R⁶ present is 0, 1, 2, 3, 4, 5 or 6, is selected independently from one another from the group consisting of hydrogen, halogen, cyano, azido, nitro, SCN, SF₅, C₁-C₁₀-alkyl, C₃-C₈-cycloalkyl, C₂-C₁₀-alkenyl, C₂-C₁₀-alkinyl, and wherein the carbon atoms of the aforementioned aliphatic and cycloaliphatic radicals may optionally be further substituted independently from one another with one or more R⁷,
      OR⁸, NR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, Si(R¹¹)₂R¹²;
      phenyl, optionally substituted with 1, 2, 3, 4 or 5 substituents selected independently from R¹⁰;
      a 3-, 4-, 5-, 6- or 7-membered saturated, partly saturated or unsaturated aromatic heterocyclic ring comprising 1, 2, 3 or 4 heteroatoms selected from oxygen, nitrogen and/or sulfur, optionally substituted with 1, 2, 3, 4, or 5 substituents selected independently from R¹⁰, and wherein the nitrogen and/or the sulfur atom(s) of the heterocyclic ring may optionally be oxidized;
      or two R⁶ present on one ring carbon atom may together form ═O, ═CR¹³R¹⁴; ═S; ═S(O)ₙR¹⁶; ═S(O)ₙNR^{17a}R^{17b}, ═NR^{17a}, ═NOR¹⁶; ═NNR^{17a};
      or two R⁶ together form a C₂-C₇ alkylene chain, thus forming, together with the ring atoms to which they are bound, a 3-, 4-, 5-, 6-, 7- or 8-membered ring, where the alkylene chain may be interrupted by 1 or 2 O, S and/or NR^{17a} and/or 1 or 2 of the CH₂ groups of the alkylene chain may be replaced by a group C═O, C═S and/or C═NR^{17a}; and/or the alkylene chain may be substituted by one or more radicals selected from the group consisting of halogen, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R¹⁸, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁸;
      or,
   R⁶, when R² is halogen and the number of R⁶ present is 1, 2, 3, 4, 5 or 6, is selected from the group consisting of, halogen, cyano, azido, nitro, SCN, SF₅, C₁-C₁₀-alkyl, C₃-C₈-cycloalkyl, C₂-C₁₀-alkenyl, C₂-C₁₀-alkinyl, and wherein the carbon atoms of the aforementioned aliphatic and cycloaliphatic radicals may optionally be further substituted independently from one another with one or more R⁷, OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, Si(R¹¹)₂R¹²;
      or two R⁶ present on one ring carbon atom may together form ═O, ═CR¹³R¹⁴; ═S; ═NR^{17a}, ═NOR¹⁶; ═NNR^{17a};
      or two R⁶ together form a C₂-C₇ alkylene chain, thus forming, together with the ring atoms to which they are bound, a 3-, 4-, 5-, 6-, 7- or 8-membered ring, where the alkylene chain may be interrupted by 1 or 2 O, S and/or NR^{17a} and/or 1 or 2 of the CH₂ groups of the alkylene chain may be replaced by a group C═O, C═S and/or C═NR^{17a}; and/or the alkylene chain may be substituted by one or more radicals selected from the group consisting of halogen, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by one or more, e.g. 1, 2, 3, 4 or 5 radicals R¹⁸, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁸;
      or
   R⁶, when R² is halogen and the number of R⁶ present is 2, 3, 4, 5 or 6, is selected from the group consisting of, halogen, cyano, azido, nitro, SCN, SF₅, C₁-C₁₀-alkyl, C₃-C₈-cycloalkyl, C₂-C₁₀-alkenyl, C₂-C₁₀-alkinyl, and wherein the carbon atoms of the aforementioned aliphatic and cycloaliphatic radicals may optionally be further substituted independently from one another with one or more R⁷, OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, Si(R¹¹)₂R¹²;
      or two R⁶ present on one ring carbon atom may together form ═O, ═CR¹³R¹⁴; ═S; ═NR^{17a}, ═NOR¹⁶; ═NNR^{17a}; phenyl, optionally substituted with one or more, e.g. 1, 2, 3, 4, or 5 substituents selected independently from R¹⁰; or
      a 3-, 4-, 5-, 6- or 7-membered saturated, partly saturated or unsaturated aromatic heterocyclic ring comprising 1, 2, 3 or 4 heteroatoms selected from oxygen, nitrogen and/or sulfur, optionally substituted with one or more, e.g. 1, 2, 3, 4, or 5 substituents selected independently from R¹⁰, and wherein the nitrogen and/or the sulfur atom(s) of the heterocyclic ring may optionally be oxidized or two R⁶ together form a C₂-C₇ alkylene chain, thus forming, together with the ring atoms to which they are bound, a 3-, 4-, 5-, 6-, 7- or 8-membered ring, where the alkylene chain may be interrupted by 1 or 2 O, S and/or NR^{17a} and/or 1 or 2 of the CH₂ groups of the alkylene chain may be replaced by a group C═O, C═S and/or C═NR^{17a}; and/or the alkylene chain may be substituted by one or more radicals selected from the group consisting of halogen, C₁-C₆-haloalkyl, C₁-C₆-alkoxy, C₁-C₆-haloalkoxy, C₁-C₆-haloalkylthio, C₃-C₆-cycloalkyl, C₃-C₆-halocycloalkyl, C₂-C₆-alkenyl, C₂-C₆-haloalkenyl, C₂-C₆-alkynyl, C₂-C₆-haloalkynyl, phenyl, which may be substituted by one or more, e.g. 1, 2, 3, 4 or 5 radicals R¹⁸, and
      a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁸;
and wherein
R⁷ is each independently from one another selected from the group consisting of hydrogen, halogen, cyano, azido, nitro, —SCN, SF₅, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₆-cycloalkyl, C₃-C₈-halocycloalkyl, C₂-C₆-alkenyl, C₂-C₆-haloalkenyl, C₂-C₆ haloalkinyl, Si(R¹¹)₂R¹², OR¹⁶, OSO₂R¹⁶, S(O)ₙR¹⁶, S(O)ₙNR^{17a}R^{17b}, NR^{17a}R^{17b}, C(═O)NR^{17a}R^{17b}, C(═S)NR^{17a}R^{17b}, C(═O)OR¹⁶, phenyl, optionally substituted with 1, 2, 3, 4 or 5 substituents R¹⁸, which are independently selected from one another,
   a 3-, 4-, 5-, 6- or 7-membered saturated, partly saturated or unsaturated aromatic heterocyclic ring comprising 1, 2 or 3 heteroatoms selected from oxygen, nitrogen and/or sulfur, optionally substituted with 1, 2, 3 or 4, substituents R¹⁸, selected independently from one another, and wherein the nitrogen and/or the sulfur atom(s) of the heterocyclic ring may optionally be oxidized,
   or
   two R⁷ present on one carbon atom may together form ═O, ═CR¹³R¹⁴; ═S; ═S(O)ₙR¹⁶; ═S(O)ₙNR^{17a}R^{17b}, ═NR^{17a}, ═NOR¹⁶; ═NNR^{17a};
   or
   two R⁷ may form a 3-, 4-, 5-, 6-, 7- or 8-membered saturated or partly unsaturated carbocyclic or heterocyclic ring together with the carbon atoms to which the two R⁷ are bonded to;
R⁸ is each independently from one another selected from the group consisting of hydrogen, cyano, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₄-C₈-alkylcycloalkyl, C₃-C₈-halocycloalkyl, C₂-C₆-alkenyl, C₂-C₆-haloalkenyl, C₂-C₆-alkinyl, C₂-C₆ haloalkinyl, —Si(R¹¹)₂R¹², S(O)ₙR¹⁶, S(O)ₙNR^{17a}R^{17b}, NR^{17a}R^{17b}, —N═CR¹³R¹⁴, —C(═O)R¹⁵, C(═O)NR^{17a}R^{17b}, C(═S)NR^{17a}R^{17b}, C(═O)OR¹⁶,
   phenyl, optionally substituted with one or more substituents R¹⁸; which are selected independently from one another,
   a 3-, 4-, 5-, 6- or 7-membered saturated, partly saturated or unsaturated aromatic heterocyclic ring comprising 1, 2 or 3 heteroatoms selected from oxygen, nitrogen and/or sulfur, optionally substituted with 1, 2, 3 or 4, substituents R¹⁸, selected independently from one another, and wherein the nitrogen and/or the sulfur atom(s) of the heterocyclic ring may optionally be oxidized;
R^{9a}, R^{9b} are each independently from one another selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl,
   S(O)ₙR¹⁶, —S(O)ₙNR^{17a}R^{17b}, C(═O)R¹⁵, C(═O)OR¹⁶, C(═O)NR^{17a}R^{17b}, C(═S)R¹⁵, C(═S)SR¹⁶, C(═S)NR^{17a}R^{17b}, C(═NR^{17a})R¹⁵;
   phenyl, optionally substituted with 1, 2, 3 or 4, substituents R¹⁸, which are selected independently from one another;
   a 3-, 4-, 5-, 6- or 7-membered saturated, partly saturated or unsaturated aromatic heterocyclic ring comprising 1, 2, 3 or 4 heteroatoms selected from oxygen, nitrogen and/or sulfur, optionally substituted with 1, 2, 3 or 4, substituents R¹⁸, selected independently from one another, and wherein the nitrogen and/or the sulfur atom(s) of the heterocyclic ring may optionally be oxidized;
   or,
   R^{9a} and R^{9b} are together a C₂-C₇ alkylene chain and form a 3-, 4-, 5-, 6-, 7- or 8-membered saturated, partly saturated or unsaturated aromatic ring together with the nitrogen atom they are bonded to, wherein the alkylene chain may contain one or two heteroatoms selected from oxygen, sulfur or nitrogen, and may optionally be substituted with halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₆-cycloalkyl, C₃-C₈-halocycloalkyl, C₂-C₆-alkenyl, C₂-C₆-haloalkenyl, C₂-C₆-alkinyl, C₂-C₆ haloalkinyl,
      phenyl, optionally substituted with one or more substituents R¹⁸; which are selected independently from one another,
      a 3-, 4-, 5-, 6,- or 7-membered saturated, partly saturated or unsaturated aromatic heterocyclic ring comprising 1, 2 or 3 heteroatoms selected from oxygen, nitrogen and/or sulfur, optionally substituted with one or more substituents R¹⁸, selected independently from one another, and wherein the nitrogen and/or the sulfur atom(s) of the heterocyclic ring may optionally be oxidized;
   or
   R^{9a} and R^{9b} together may form a ═CR¹³R¹⁴, ═S(O)ₙ(R¹⁶)₂, ═NR¹⁷ or ═NOR¹⁶ radical;
R¹⁰ is each independently from one another selected from the group consisting of hydrogen, halogen, cyano, azido, nitro, SCN, SF₅, C₁-C₁₀-alkyl, C₃-C₆-cycloalkyl, C₂-C₁₀-alkenyl, C₂-C₁₀-alkinyl, wherein the carbon atoms of the aforementioned aliphatic and cycloaliphatic radicals may optionally be substituted with one or more R¹⁵, which are selected independently from one another,
   Si(R¹¹)₂R¹², OR¹⁶, OS(O)ₙR¹⁶, —S(O)ₙR¹⁶, S(O)ₙNR^{17a}R^{17b}, NR^{17a}R^{17b}, C(═O)R¹⁵, C(═O)OR¹⁶, —C(═NR^{17a})R¹⁵, C(═O)NR^{17a}R^{17b}, C(═S)NR^{17a}R^{17b}, phenyl, optionally substituted with halogen, cyano, nitro, C₁-C₆-alkyl, C₁-C₆-haloalkyl, C₁-C₆-alkoxy or C₁-C₆-haloalkoxy,
   a 3-, 4-, 5-, 6- or 7-membered saturated, partly saturated or unsaturated aromatic heterocyclic ring comprising 1, 2 or 3 heteroatoms selected from oxygen, nitrogen and/or sulfur, optionally substituted with one or more substituents selected independently from one another from halogen, cyano, NO₂, C₁-C₆-alkyl, C₁-C₆-haloalkyl, C₁-C₆-alkoxy or C₁-C₆-haloalkoxy, and wherein the nitrogen and/or the sulfur atom(s) of the heterocyclic ring may optionally be oxidized;
   or
   two R¹⁰ present together on one atom of a partly saturated heterocyclic may be ═O, ═CR¹³R¹⁴; ═NR^{17a}, ═NOR¹⁶ or ═NNR^{17a}; or, two R¹⁰ on adjacent carbon atoms may be a bridge selected from CH₂CH₂CH₂CH₂, CH═CH—CH═CH, N═CH—CH═CH, CH═N—CH═CH, N═CH—N═CH, OCH₂CH₂CH₂, OCH═CHCH₂, CH₂OCH₂CH₂, OCH₂CH₂O, OCH₂OCH₂, CH₂CH₂CH₂, CH═CHCH₂, CH₂CH₂O, CH═CHO, CH₂OCH₂, CH₂C(═O)O, C(═O)OCH₂, O(CH₂)O, SCH₂CH₂CH₂, SCH═CHCH₂, CH₂SCH₂CH₂, SCH₂CH₂S, SCH₂SCH₂, CH₂CH₂S, CH═CHS, CH₂SCH₂, CH₂C(═S)S, C(═S)SCH₂, S(CH₂)S, CH₂CH₂NR^{17a}, CH₂CH═N, CH═CH—NR^{17a}, OCH═N, SCH═N and form together with the carbon atoms to which the two R¹⁰ are bonded to a 5-membered or 6-membered partly saturated or unsaturated, aromatic carbocyclic or heterocyclic ring, wherein the ring may optionally be substituted with one or two substituents selected from ═O, OH, CH₃, OCH₃, halogen, cyano, halomethyl or halomethoxy;
R¹¹, R¹² are each independently from one another selected from the group consisting of hydrogen, halogen, C₁-C₆ alkyl, C₁-C₆ haloalkyl, C₁-C₆ alkoxy, C₁-C₆ alkoxyalkyl, C₂-C₆ alkenyl, C₂-C₆ haloalkenyl, C₂-C₆ alkinyl, C₂-C₆ haloalkinyl, C₃-C₆ cycloalkyl, C₃-C₆ halocycloalkyl, C₁-C₆ alkoxyalkyl, C₁-C₆ haloalkoxyalkyl and
   phenyl, optionally substituted with one or more substituents R¹⁸;
   which are selected independently from one another;
R¹³, R¹⁴ are each independently from one another selected from the group consisting of hydrogen, C₁-C₄ alkyl, C₁-C₆ cycloalkyl, C₁-C₄ alkoxyalkyl, phenyl and benzyl;
R¹⁵ is each independently from one another selected from the group consisting of hydrogen, halogen, cyano, nitro, OH, SH, SCN, SF₅, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl, C₁-C₆-alkyl, C₂-C₆-alkenyl, C₂-C₆-alkinyl, C₃-C₈-cycloalkyl, wherein the four last mentioned aliphatic and cycloaliphatic radicals may be unsubstituted, partially or fully halogenated and/or oxgenated and/or may carry 1 or 2 radicals selected from C₁-C₄ alkoxy;
   phenyl, benzyl, pyridyl, phenoxy, wherein the last four radicals may be unsubstituted, partially or fully halogenated and/or to carry 1, 2 or 3 substituents selected from C₁-C₆-alkyl, C₁-C₆-haloalkyl, C₁-C₆-alkoxy, C₁-C₆ haloalkoxy, (C₁-C₆-alkoxy)carbonyl, (C₁-C₆-alkyl)amino or di-(C₁-C₆-alkyl)amino, or
   two R¹⁵ present on the same carbon atom may together be ═O, ═CH(C₁-C₄), ═C(C₁-C₄-alkyl)C₁-C₄-alkyl, ═N(C₁-C₆-alkyl) or ═NO(C₁-C₆-alkyl);
R¹⁶ is each independently from one another selected from the group consisting of hydrogen, cyano, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl,
   C₁-C₆-alkyl, C₂-C₆-alkenyl, C₂-C₆-alkinyl, C₃-C₈-cycloalkyl, wherein the four last mentioned radicals may be unsubstituted, partially or fully halogenated and/or oxygenated and/or may carry 1 or 2 radicals selected from C₁-C₄ alkoxy,
   phenyl, benzyl, pyridyl, phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy or (C₁-C₆-alkoxy)carbonyl;
R^{17a}, R^{17b} are each independently from one another selected from the group consisting of hydrogen, cyano, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl,
   C₁-C₆-alkyl, C₂-C₆-alkenyl, C₂-C₆-alkinyl, C₃-C₈-cycloalkyl, wherein the four last mentioned aliphatic and cycloaliphatic radicals may be unsubstituted, partially or fully halogenated and/or oxygenated and/or may carry 1 or 2 radicals selected from C₁-C₄-alkoxy,
   phenyl, benzyl, pyridyl, phenoxy, wherein the four last mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy or (C₁-C₆-alkoxy)carbonyl, or,
   R^{17a} and R^{17b} may together be a C₂-C₆ alkylene chain forming a 3- to 7-membered saturated, partly saturated or unsaturated ring together with the nitrogen atom R^{17a} and R^{17b} are bonded to, wherein the alkylene chain may contain 1 or 2 heteroatoms selected from oxygen, sulfur or nitrogen, and may optionally be substituted with halogen, C₁-C₄-haloalkyl, C₁-C₄-alkoxy or C₁-C₄-haloalkoxy, and wherein the nitrogen and/or the sulfur atom(s) of the heterocyclic ring may optionally be oxidized;
R¹⁸ is each independently from one another selected from the group consisting of hydrogen, halogen, nitro, cyano, OH, SH, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl, C₁-C₆-alkyl, C₂-C₆-alkenyl, C₂-C₆-alkinyl, C₃-C₈-cycloalkyl, wherein the four last mentioned aliphatic and cycloaliphatic radicals may be unsubstituted, partially or fully halogenated and/or oxygenated and/or may carry 1 or 2 radicals selected from C₁-C₄-alkoxy,
   phenyl, benzyl, pyridyl, phenoxy, wherein the four last mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy); (C₁-C₆-alkoxy)carbonyl;
   or
   two R¹⁸ present together on one atom of a partly saturated atom may be ═O, ═S, ═N(C₁-C₆-alkyl), ═NO(C₁-C₆-alkyl), ═CH(C₁-C₄-alkyl) or ═C(C₁-C₄-alkyl)C₁-C₄-alkyl;
   or,
   two R¹⁸ on two adjacent carbon atoms may be together a C₂-C₆ alkylene chain, which form together with the carbon atom they are bonded to a 3-, 4-, 5-, 6- or 7-membered saturated, partly saturated or unsaturated aromatic, wherein the alkylene chain may contain 1 or 2 heteroatoms selected from oxygen, sulfur or nitrogen, and may optionally be substituted with halogen, C₁-C₄-alkoxy or C₁-C₄-haloalkoxy, and wherein the nitrogen and/or the sulfur atom(s) of the heterocyclic ring may optionally be oxidized;
n is 0, 1 or 2;
and/or an enantiomer, diastereomer or agriculturally or veterinarily acceptable salts thereof.

One embodiment of the present invention is a method for combating or controlling invertebrate pests comprising contacting the invertebrate pests, or their food supply, habitat or breeding grounds with a substituted 3-pyridyl thiazole compound of the general formula (I) as defined above or a composition comprising at least one compound of formula (I) as defined above.

One embodiment of the present invention is a method for protecting crops, plants, plant propagation material and/or growing plants from attack or infestation by invertebrate pests comprising contacting or treating the crops, plants, plant propagation material and growing plants, or soil, material, surface, space, area or water in which the crops, plants, plant propagation material is stored or the plant is growing, with a substituted 3-pyridyl thiazole compound of the general formula (I) as defined above or a composition comprising at least one compound of formula (I) as defined above.

One embodiment of the present invention is a method for treating, controlling, preventing or protecting animals against infestation or infection by parasites by administering or applying orally, topically or parenterally to the animals a substituted 3-pyridyl thiazole compound of the general formula (I) as defined above or a composition comprising at least one compound of formula (I) as defined above.

In another aspect, the present inventions relates to pesticidal substituted 3-pyridyl thiazole compounds of formula (I)

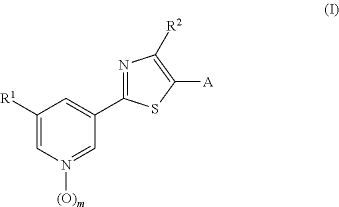

wherein m, A, R¹ and R² are as defined above;
and/or an enantiomer, diastereomer or agriculturally or veterinarily acceptable salts thereof.

Furthermore, the invention relates to processes for the synthesis of compounds of formula (I) according to the present invention and to intermediate compounds for the synthesis of compounds of formula (I).

One embodiment of the present invention is an intermediate compound of the formula (I-4)

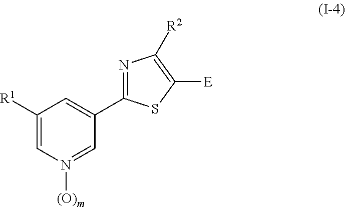

wherein
m is 0 or 1;
R¹ is selected from the group consisting of hydrogen or fluoro;
R² is selected from the group consisting of halogen or C₁-C₆-haloalkyl, the latter may be partially or fully halogenated and may optionally be further substituted by 1, 2, 3 or 4 radicals R⁷ as defined above;
E is selected from Cl, Br, I, the molecular group E1 or the molecular group E2, wherein
   the molecular group E1 is

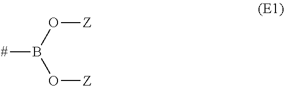

wherein # denotes the bond to the thiazole ring in formula (I-4), and each Z is independently from one another hydrogen or C₁-C₄ alkyl; and
the molecular group (E2) is

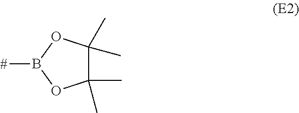

wherein # denotes the bond to the thiazole ring in formula (I-4).

Another embodiment of the present invention is a process for the preparation of compounds of formula (I), wherein an intermediate compound of formula (I-4) is used.

The compounds of the present invention, i.e. the compounds of formula (I), their stereoisomers, their salts or their N-oxides, are particularly useful for controlling invertebrate pests, in particular for controlling arthropods and nematodes and especially insects. Therefore, the invention relates to the use of a compound of the present invention, for combating or controlling invertebrate pests, in particular invertebrate pests of the group of insects, arachnids or nematodes.

The term “compound(s) according to the invention” or “compound(s) of formula (I)” comprises the compound(s) as defined herein as well as a stereoisomer, salt, tautomer or N-oxide thereof. The term “compound(s) of the present invention” is to be understood as equivalent to the term “compound(s) according to the invention”, therefore also comprising a stereoisomer, salt, tautomer or N-oxide thereof.

The term “composition(s) according to the invention” or “composition(s) of the present invention” encompasses composition(s) comprising at least one compound of formula (I) according to the invention as defined above.

The present invention relates to a composition comprising at least one compound according to the invention, including a stereoisomer, salt, tautomer or N-oxide thereof, and at least one inert liquid and/or solid carrier. In particular, the invention relates to an agricultural or veterinary composition comprising at least one compound according to the invention including a stereoisomer, an agriculturally or veterinarily acceptable salt, tautomer or an N-oxide thereof, and at least one liquid and/or solid carrier.

The present invention relates to a method for combating or controlling invertebrate pests of the group of insects, arachnids or nematodes, which method comprises contacting said pest or its food supply, habitat or breeding grounds with a pesticidally effective amount of at least one compound according to the invention including a stereoisomer, salt, tautomer or N-oxide thereof or a composition according to the invention.

The present invention also relates to a method for protecting growing plants from attack or infestation by invertebrate pests of the group of insects, arachnids or nematodes, which method comprises contacting a plant, or soil or water in which the plant is growing or may grow, with a pesticidally effective amount of at least one compound according to the invention including a stereoisomer, salt, tautomer or N-oxide thereof or a composition according to the invention.

The present invention also relates to a method for the protection of plant propagation material, preferably seeds, from soil insects and of the seedlings' roots and shoots from soil and foliar insects comprising contacting the seeds before sowing and/or after pregermination with at least one compound according to the invention including a stereoisomer, salt, tautomer or N-oxide thereof or a composition according to the invention.

The present invention also relates to plant propagation material, preferably seed, comprising a compound according to the invention including a stereoisomer, salt, tautomer or N-oxide thereof.

The present invention also relates to the use of a compound according to the invention including a stereoisomer, salt, tautomer or N-oxide thereof or a composition according to the invention for combating or controlling invertebrate pests of the group of insects, arachnids or nematodes.

The present invention also relates to the use of a compound according to the invention including a stereoisomer, salt or N-oxide thereof or a composition according to the invention for protecting growing plants from attack or infestation by invertebrate pests of the group of insects, arachnids or nematodes.

The present invention also relates to the use of a compound according to the invention including a stereoisomer, veterinarily acceptable salt, tautomer or N-oxide thereof or a composition according to the invention for combating or controlling invertebrate parasites in and on animals.

The present invention also relates to a method for treating an animal infested or infected by parasites or for preventing animals from getting infested or infected by parasites or for protecting an animal against infestation or infection by parasites which comprises orally, topically or parenterally administering or applying to the animal a parasiticidally effective amount of a compound according to the invention including a stereoisomer, veterinarily acceptable salt, tautomer or N-oxide thereof or a composition according to the invention.

The present invention also relates to the use of a compound according to the invention including a stereoisomer, veterinarily acceptable salt or N-oxide thereof or a composition according to the invention for the manufacture of a medicament for protecting an animal against infestation or infection by parasites or treating an animal infested or infected by parasites.

The present invention also relates to a process for the preparation of a composition for treating animals infested or infected by parasites, for preventing animals of getting infected or infested by parasites or protecting animals against infestation or infection by parasites which comprises a compound according to the invention including a stereoisomer, veterinarily acceptable salt, tautomer or N-oxide thereof.

The present invention also relates to a compound according to the invention including a stereoisomer, veterinarily acceptable salt, tautomer or N-oxide thereof for use as a veterinary medicament.

The present invention also relates to a compound according to the invention including a stereoisomer, veterinarily acceptable salt, tautomer or N-oxide thereof for use in the treatment, control, prevention or protection of animals against infestation or infection by parasites.

Substituted 3-pyridyl thiazole compounds according to the present invention have not yet been described for pesticidal uses or pesticidal applications in agricultural industry or veterinary practice.

Certain diaryl-thiazoles and substituted pyridyl thiazole heterocycles are disclosed in Bioorganic & Medicinal Chemistry Letters 2012, 22(9), 3083-3088, in WO 2012021696, in WO 2011133733, in EP 117082, in EP 149884 and WO 2001010865 as enzyme inhibitors and pharmaceutical agents.

None of these documents discloses substituted 3-pyridyl thiazole compounds showing insectividal activity or their use insecticidal methods.

WO 2010006713 describes pyridyl thiazole-subsituted heterocycle derivatives and their use as pesticides. However, this document does not disclose halogen- or haloalkyl substituted thiazoles as in this present invention. Other pyridyl thiazole-subsituted heterocycle pesticidal compounds are likewise disclosed in WO 2011134964, WO 2011138285 and WO 2012000896.

WO 2010129497 describes pyridyl thiazole amines and their applications as pesticides. Similar pesticidal compounds are likewise disclosed in WO 2011128304 and WO 2012030681. Pesticidal 3-pyridyl thiazole carboxamides have been described in the U.S. Pat. No. 4,260,765. WO 2009149858 describes pyridyl thiazole carboxamide derivatives and their applications as pesticide. Similar pesticidal carboxamide compounds are likewise disclosed in WO 2011128304.

4-haloalkyl-3-heterocyclylpyridines as pesticides are disclosed in WO 9857969. Similar compounds are likewise disclosed in WO 2000035285 and US 20030162812. Heterocyclyl-substituted thiazole derivatives and their use as fungicides have been described in WO 2007033780. Substituted haloalkyl thiazole derivatives and their use as insecticides are disclosed in WO 2004056177.

However, substituted 3-pyridyl thiazole compounds with the characteristic substitution pattern as in this present invention have not yet been described.

Depending on the substitution pattern, the compounds of the formula (I) may have one or more centers of chirality, in which case they are present as mixtures of enantiomers or diastereomers. The invention provides both the single pure enantiomers or pure diastereomers of the compounds of formula (I), and their mixtures and the use according to the invention of the pure enantiomers or pure diastereomers of the compound of formula (I) or its mixtures. Suitable compounds of the formula (I) also include all possible geometrical stereoisomers (cis/trans isomers) and mixtures thereof. Cis/trans isomers may be present with respect to an alkene, carbon-nitrogen double-bond or amide group. The term “stereoisomer(s)” encompasses both optical isomers, such as enantiomers or diastereomers, the latter existing due to more than one center of chirality in the molecule, as well as geometrical isomers (cis/trans isomers). The present invention relates to every possible stereoisomer of the compounds of formula I, i.e. to single enantiomers or diastereomers, as well as to mixtures thereof.

Depending on the substitution pattern, the compounds of the formulae (I) may be present in the form of their tautomers. Hence the invention also relates to the tautomers of the formula (I) and the stereoisomers, salts, tautomers and N-oxides of said tautomers. The compounds of the present invention may be amorphous or may exist in one ore more different crystalline states (polymorphs) or modifications which may have a different macroscopic properties such as stability or show different biological properties such as activities. The present invention includes both amorphous and crystalline compounds of the formula I, mixtures of different crystalline states or modifications of the respective compound I, as well as amorphous or crystalline salts thereof.

Salts of the compounds of the formula I are preferably agriculturally and/or veterinary acceptable salts. They can be formed in a customary method, e.g. by reacting the compound with an acid of the anion in question if the compound of formula I has a basic functionality or by reacting an acidic compound of formula I with a suitable base.

Suitable agriculturally or veterinary useful salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethyl-ammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting the compounds of the formulae I with an acid of the corresponding anion, preferably of hydrochloric acid, hydrobromic acid, sulfuric acid, phosphoric acid or nitric acid.

The term “N-oxide” includes any compound of the present invention which has at least one tertiary nitrogen atom that is oxidized to an N-oxide moiety.

The organic moieties mentioned in the above definitions of the variables are—like the term halogen—collective terms for individual listings of the individual group members. The prefix Cₙ-Cₘ indicates in each case the possible number of carbon atoms in the group.

“Halogen” will be taken to mean fluoro, chloro, bromo and iodo.

The term “partially or fully halogenated” will be taken to mean that 1 or more, e.g. 1, 2, 3, 4 or 5 or all of the hydrogen atoms of a given radical have been replaced by a halogen atom, in particular by fluorine or chlorine.

The term “Cₙ-Cₘ-alkyl” as used herein (and also in Cₙ-Cₘ-alkylamino, Cₙ-Cₘ-alkylaminocarbonyl, di-(Cₙ-Cₘ-alkylamino)carbonyl, Cₙ-Cₘ-alkylthio, Cₙ-Cₘ-alkylsulfinyl and Cₙ-Cₘ-alkylsulfonyl) refers to a branched or unbranched saturated hydrocarbon group having n to m, e.g. 1 to 10 carbon atoms, preferably 1 to 6 carbon atoms, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, nonyl and decyl and their isomers. C₁-C₄-alkyl means for example methyl, ethyl, propyl, 1-methylethyl, butyl, 1-methylpropyl, 2-methylpropyl or 1,1-dimethylethyl.

The term “Cₙ-Cₘ-haloalkyl” as used herein (and also in Cₙ-Cₘ-haloalkylsulfinyl and Cₙ-Cₘ-haloalkylsulfonyl) refers to a straight-chain or branched alkyl group having n to m carbon atoms, e.g. 1 to 10 in particular 1 to 6 carbon atoms (as mentioned above), where some or all of the hydrogen atoms in these groups may be replaced by halogen atoms as mentioned above, for example C₁-C₄-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like. The term C₁-C₁₀-haloalkyl in particular comprises C₁-C₂-fluoroalkyl, which is synonym with methyl or ethyl, wherein 1, 2, 3, 4 or 5 hydrogen atoms are substituted by fluorine atoms, such as fluoromethyl, difluoromethyl, trifluoromethyl, 1-fluoroethyl, 2-fluoroethyl, 2,2-difluoroethyl, 2,2,2-trifluoroethyl and pentafluoromethyl.

Similarly, “Cₙ-Cₘ-alkoxy” and “Cₙ-Cₘ-alkylthio” (or Cₙ-Cₘ-alkylsulfenyl, respectively) refer to straight-chain or branched alkyl groups having n to m carbon atoms, e.g. 1 to 10, in particular 1 to 6 or 1 to 4 carbon atoms (as mentioned above) bonded through oxygen or sulfur linkages, respectively, at any bond in the alkyl group. Examples include C₁-C₄-alkoxy such as methoxy, ethoxy, propoxy, isopropoxy, butoxy, sec-butoxy, isobutoxy and tert-butoxy, further C₁-C₄-alkylthio such as methylthio, ethylthio, propylthio, isopropylthio, and n-butylthio.

Accordingly, the terms “Cₙ-Cₘ-haloalkoxy” and “Cₙ-Cₘ-haloalkylthio” (or Cₙ-Cₘ-haloalkylsulfenyl, respectively) refer to straight-chain or branched alkyl groups having n to m carbon atoms, e.g. 1 to 10, in particular 1 to 6 or 1 to 4 carbon atoms (as mentioned above) bonded through oxygen or sulfur linkages, respectively, at any bond in the alkyl group, where some or all of the hydrogen atoms in these groups may be replaced by halogen atoms as mentioned above, for example C₁-C₂-haloalkoxy, such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chloro-difluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy and pentafluoroethoxy, further C₁-C₂-haloalkylthio, such as chloromethylthio, bromomethylthio, dichloro-methylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio and pentafluoroethylthio and the like. Similarly the terms C₁-C₂-fluoroalkoxy and C₁-C₂-fluoroalkylthio refer to C₁-C₂-fluoroalkyl which is bound to the remainder of the molecule via an oxygen atom or a sulfur atom, respectively.

The term “C₂-Cₘ-alkenyl” as used herein intends a branched or unbranched unsaturated hydrocarbon group having 2 to m, e.g. 2 to 10 or 2 to 6 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term “C₂-Cₘ-alkynyl” as used herein refers to a branched or unbranched unsaturated hydrocarbon group having 2 to m, e.g. 2 to 10 or 2 to 6 carbon atoms and containing at least one triple bond, such as ethynyl, propynyl, 1-butynyl, 2-butynyl, and the like.

The term “C₁-C₄-alkoxy-C₁-C₄-alkyl” as used herein refers to alkyl having 1 to 4 carbon atoms, e.g. like specific examples mentioned above, wherein one hydrogen atom of the alkyl radical is replaced by an C₁-C₄-alkoxy group.

The term “C₃-Cₘ-cycloalkyl” as used herein refers to a monocyclic 3- to m-membered saturated cycloaliphatic radicals, e.g. cyclopropyl, cyclobutyl, cyclopentyl, cyclohexyl, cycloheptyl, cyclooctyl and cyclodecyl.

The term “aryl” as used herein refers to an aromatic hydrocarbon radical such as naphthyl or in particular phenyl.

The term “3- to 6-membered carbocyclic ring” as used herein refers to cyclopropane, cyclobutane, cyclopentane and cyclohexane rings.

The term “3-, 4-, 5-, 6- or 7-membered saturated, partially unsaturated or aromatic heterocyclic ring containing 1, 2 or 3 heteroatoms” or “containing heteroatom groups”, wherein those heteroatom(s) (group(s)) are selected from N, O, S, NO, SO and SO₂ and are ring members, as used herein refers to monocyclic radicals, the monocyclic radicals being saturated, partially unsaturated or aromatic. The heterocyclic radical may be attached to the remainder of the molecule via a carbon ring member or via a nitrogen ring member.

Examples of 3-, 4-, 5-, 6- or 7-membered saturated heterocyclyl or heterocyclic rings include: Oxiranyl, aziridinyl, azetidinyl, 2 tetrahydrofuranyl, 3-tetrahydrofuranyl, 2 tetrahydrothienyl, 3 tetrahydrothienyl, 2-pyrrolidinyl, 3-pyrrolidinyl, 3 pyrazolidinyl, 4 pyrazolidinyl, 5-pyrazolidinyl, 2 imidazolidinyl, 4 imidazolidinyl, 2-oxazolidinyl, 4-oxazolidinyl, 5 oxazolidinyl, 3-isoxazolidinyl, 4 isoxazolidinyl, 5 isoxazolidinyl, 2 thiazolidinyl, 4-thiazolidinyl, 5-thiazolidinyl, 3 isothiazolidinyl, 4-isothiazolidinyl, 5 isothiazolidinyl, 1,2,4-oxadiazolidin-3-yl, 1,2,4 oxadiazolidin 5 yl, 1,2,4-thiadiazolidin-3-yl, 1,2,4 thiadiazolidin-5-yl, 1,2,4 triazolidin-3-yl, 1,3,4-oxadiazolidin-2-yl, 1,3,4 thiadiazolidin-2-yl, 1,3,4 triazolidin-2-yl, 2-tetrahydropyranyl, 4 tetrahydropyranyl, 1,3-dioxan-5-yl, 1,4-dioxan-2-yl, 2-piperidinyl, 3-piperidinyl, 4-piperidinyl, 3-hexahydropyridazinyl, 4 hexahydropyridazinyl, 2-hexahydropyrimidinyl, 4-hexahydropyrimidinyl, 5 hexahydropyrimidinyl, 2-piperazinyl, 1,3,5-hexahydrotriazin-2-yl and 1,2,4 hexahydrotriazin-3-yl, 2-morpholinyl, 3-morpholinyl, 2-thiomorpholinyl, 3-thiomorpholinyl, 1-oxothiomorpholin-2-yl, 1-oxothiomorpholin-3-yl, 1,1-dioxothiomorpholin-2-yl, 1,1-dioxothiomorpholin-3-yl, hexa-hydroazepin-1-, -2-, -3- or -4-yl, hexahydrooxepinyl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl and the like.

Examples of 3-, 4-, 5-, 6- or 7-membered partially unsaturated heterocyclyl or heterocyclic rings include: 2,3-dihydrofur-2-yl, 2,3-dihydrofur-3-yl, 2,4-dihydrofur-2-yl, 2,4-dihydrofur-3-yl, 2,3-dihydrothien-2-yl, 2,3 dihydrothien-3-yl, 2,4 dihydrothien-2-yl, 2,4-dihydrothien-3-yl, 2-pyrrolin-2-yl, 2-pyrrolin-3-yl, 3 pyrrolin-2-yl, 3-pyrrolin-3-yl, 2-isoxazolin-3-yl, 3-isoxazolin-3-yl, 4 isoxazolin 3 yl, 2-isoxazolin-4-yl, 3-isoxazolin-4-yl, 4-isoxazolin-4-yl, 2 isoxazolin-5-yl, 3-isoxazolin-5-yl, 4-isoxazolin-5-yl, 2-isothiazolin-3-yl, 3 isothiazolin-3-yl, 4-isothiazolin-3-yl, 2-isothiazolin-4-yl, 3-isothiazolin-4-yl, 4 isothiazolin-4-yl, 2-isothiazolin-5-yl, 3-isothiazolin-5-yl, 4-isothiazolin-5-yl, 2,3 dihydropyrazol-1-yl, 2,3-dihydropyrazol-2-yl, 2,3-dihydropyrazol-3-yl, 2,3 dihydropyrazol-4-yl, 2,3-dihydropyrazol-5-yl, 3,4-dihydropyrazol-1-yl, 3,4 dihydropyrazol-3-yl, 3,4-dihydropyrazol-4-yl, 3,4-dihydropyrazol-5-yl, 4,5 dihydropyrazol-1-yl, 4,5-dihydropyrazol-3-yl, 4,5-dihydropyrazol-4-yl, 4,5 dihydropyrazol-5-yl, 2,3-dihydrooxazol-2-yl, 2,3-dihydrooxazol-3-yl, 2,3 dihydrooxazol-4-yl, 2,3-dihydrooxazol-5-yl, 3,4-dihydrooxazol-2-yl, 3,4 dihydrooxazol-3-yl, 3,4-dihydrooxazol-4-yl, 3,4-dihydrooxazol-5-yl, 3,4 dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4 di- or tetrahydropyridazinyl, 2-di- or tetrahydropyrimidinyl, 4-di- or tetrahydropyrimidinyl, 5 di- or tetrahydropyrimidinyl, di- or tetrahydropyrazinyl, 1,3,5-di- or tetrahydrotriazin-2-yl, 1,2,4-di- or tetrahydrotriazin-3-yl, 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7 tetrahydro[1H]azepin-1-, -2-, -3-, -4-, -5-, -6- or -7-yl, 2,3,6,7 tetrahydro[1H]azepin-1-, -2-, -3-, -4-, -5-, -6- or -7-yl, tetrahydrooxepinyl, such as 2,3,4,5-tetrahydro[1H]oxepin-2-, -3-, -4-, -5-, -6- or -7-yl, 2,3,4,7 tetrahydro[1H]oxepin-2-, -3-, -4-, -5-, -6- or -7-yl, 2,3,6,7 tetrahydro[1H]oxepin-2-, -3-, -4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl and tetrahydro-1,4-dioxepinyl.

Examples of 5- or 6-membered aromatic heterocyclyl (hetaryl) or heteroaromatic rings are: 2-furyl, 3-furyl, 2-thienyl, 3-thienyl, 2-pyrrolyl, 3-pyrrolyl, 3-pyrazolyl, 4-pyrazo [symbol] lyl, 5-pyrazolyl, 2-oxazolyl, 4-oxazolyl, 5-oxazolyl, 2-thiazolyl, 4 thiazolyl, 5-thiazo [symbol] lyl, 2-imidazolyl, 4-imidazolyl, 1,3,4-triazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

A “C₂-Cₘ-alkylene” is divalent branched or preferably unbranched saturated aliphatic chain having 2 to m, e.g. 2 to 7 carbon atoms, for example CH₂CH₂, —CH(CH₃)—, CH₂CH₂CH₂, CH(CH₃)CH₂, CH₂CH(CH₃), CH₂CH₂CH₂CH₂, CH₂CH₂CH₂CH₂CH₂, CH₂CH₂CH₂CH₂CH₂CH₂, and CH₂CH₂CH₂CH₂CH₂CH₂CH₂.

### Preferences

Embodiments and preferred compounds of the present invention for use in pesticidal methods and for insecticidal application purposes are outlined in the following paragraphs.

The description concerning the preferred substituents and the remarks made below concerning preferred embodiments of the variables of the compounds of formula I, especially with respect to their substituents A, R¹ and R² are valid both on their own and, in particular, in every possible combination with each other.

These preferences apply to the pesticidal compounds of formula (I) as such, as well, as to the methods using such preferred compounds.

Preferred are substituted 3-pyridyl thiazole compounds of the general formula (I) of the present invention, wherein

R¹ is selected from the group consisting of hydrogen or fluoro.

Preferred are substituted 3-pyridyl thiazole compounds of the general formula (I) of the present invention, wherein
R² is selected from the group consisting of partially or fully halogenated C₁-C₄ haloalkyl, wherein the C₁-C₄ haloalkyl is not further substituted with R⁷.

Especially preferred are substituted 3-pyridyl thiazole compounds of the general formula (I) of the present invention, wherein
R¹ is selected from the group consisting of hydrogen or fluoro;
and
R² is selected from the group consisting of CHF₂, CF₃, CHCl₂, CCl₃ and C₂-C₄ haloalkyl.

Preferred are substituted 3-pyridyl thiazole compounds of the general formula (I-3) of the present invention.

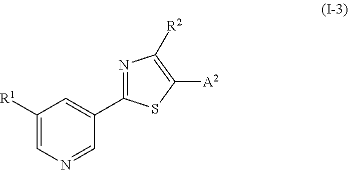

### Wherein

R² is selected from the group consisting of partially or fully halogenated C₁-C₄ haloalkyl;
A² is a nitrogen containing 4-, 5-, or 6-membered heterocyclic or hetero-aromatic ring system, optionally substituted with 1, 2, 3, 4, 5 or 6 substituents R⁶, which are selected independently from one another, and wherein
   R⁶ is independently selected from the group consisting of hydrogen, halogen, cyano, nitro, SCN, C₁-C₆-alkyl, C₃-C₆-cycloalkyl, C₂-C₆-alkenyl, C₂-C₆-alkinyl, wherein the aforementioned aliphatic and cycloaliphatic radicals may optionally be substituted with one or more R⁷, which are selected independently from one another,
      OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b};
      phenyl, optionally substituted with one or more, e.g. 1, 2, 3, 4, or 5 substituents selected independently from R¹⁰;
      a 3-, 4-, 5-, 6- or 7-membered saturated, partly saturated or unsaturated aromatic heterocyclic ring comprising 1, 2, 3 or 4 heteroatoms selected from oxygen, nitrogen and/or sulfur, optionally substituted with one or more, e.g. 1, 2, 3, 4, or 5 substituents selected independently from R¹⁰, and wherein the nitrogen and/or the sulfur atom(s) of the heterocyclic ring may optionally be oxidized;
      or
      two R⁶ present on one ring carbon atom may together form ═O, ═S, ═NR^{17a}.

Especially more preferred are substituted 3-pyridyl thiazole compounds of the general formula (I-3) of the present invention, wherein
R¹ is selected from the group consisting of hydrogen or fluoro;
R² is selected from the group consisting of CHF₂, CF₃, CHCl₂, CCl₃ and C₂-C₄ haloalkyl;
A² is a nitrogen containing 4-, 5-, or 6-membered heterocyclic or hetero-aromatic ring system, optionally substituted with one or more, e.g. 1, 2, 3, 4, 5, or 6 substituents R⁶, which are selected independently from one another; and wherein
   R⁶ is independently selected from the group consisting of hydrogen, halogen, cyano, nitro, SCN, C₁-C₆-alkyl, C₃-C₆-cycloalkyl, C₂-C₆-alkenyl, C₂-C₆-alkinyl, wherein the aforementioned aliphatic and cycloaliphatic radicals may optionally be substituted with one or more R⁷, which are selected independently from one another,
      OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b};
      phenyl, optionally substituted with one or more, e.g. 1, 2, 3, 4, or 5 substituents selected independently from R¹⁰;
      a 3-, 4-, 5-, 6- or 7-membered saturated, partly saturated or unsaturated aromatic heterocyclic ring comprising 1, 2, 3 or 4 heteroatoms selected from oxygen, nitrogen and/or sulfur, optionally substituted with one or more, e.g. 1, 2, 3, 4, or 5 substituents selected independently from R¹⁰, and wherein the nitrogen and/or the sulfur atom(s) of the heterocyclic ring may optionally be oxidized;
      or
      two R⁶ present on one ring carbon atom may together form ═O, ═S, ═NR^{17a};

Especially preferred are substituted 3-pyridyl thiazole compounds of the general formula (I) of the present invention, wherein
A² is a nitrogen containing 4-, 5-, or 6-membered heterocyclic or hetero-aromatic ring system selected from any of the following ring systems D-1-D-135:

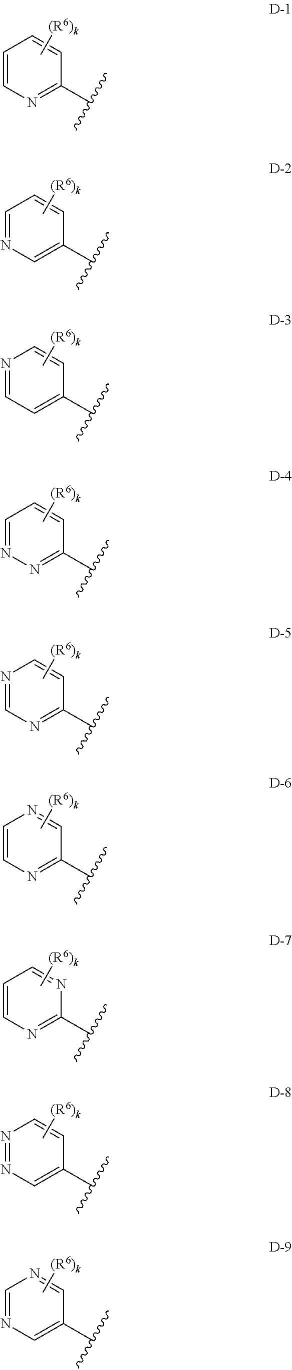

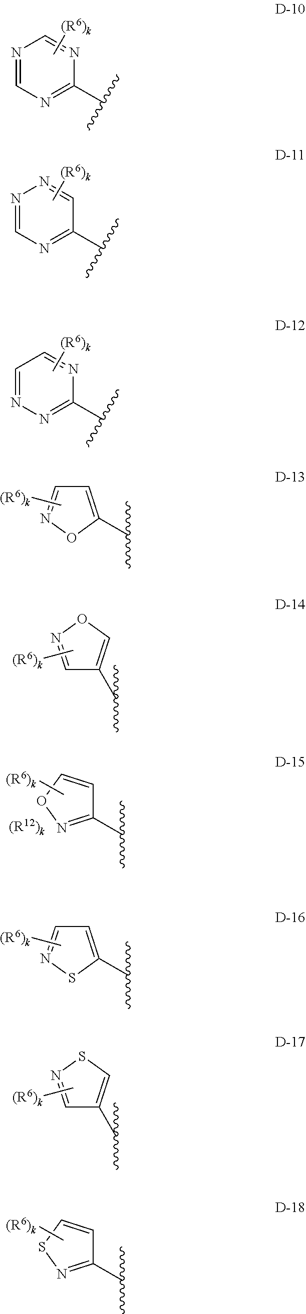

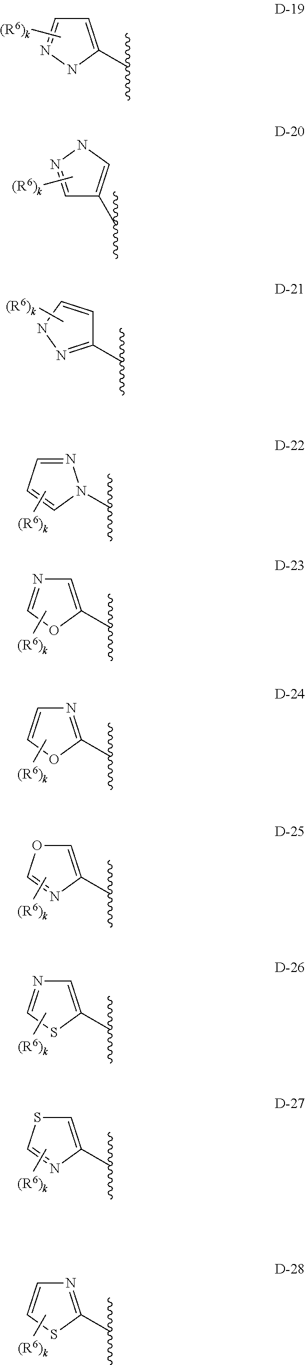

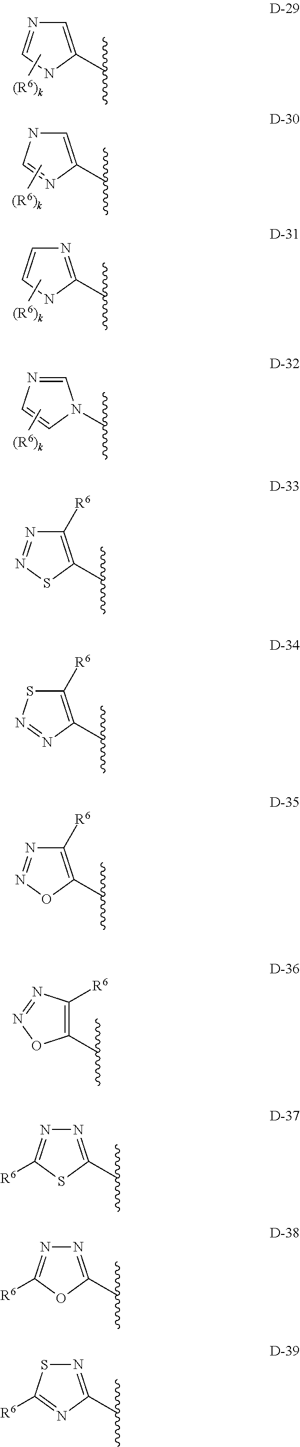

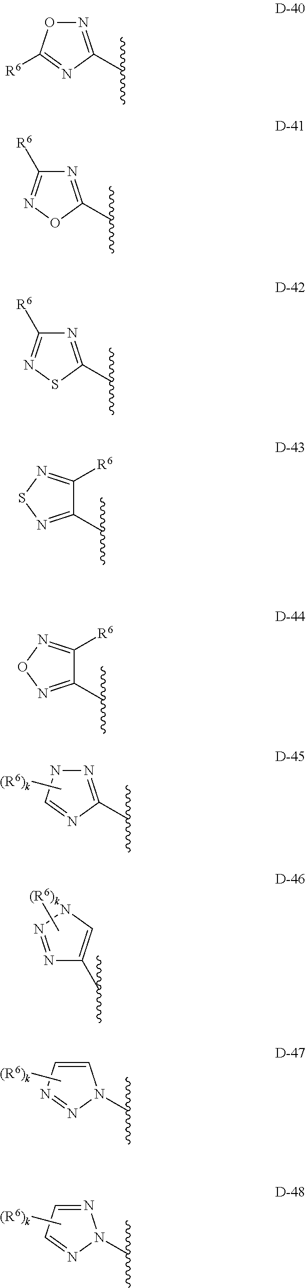

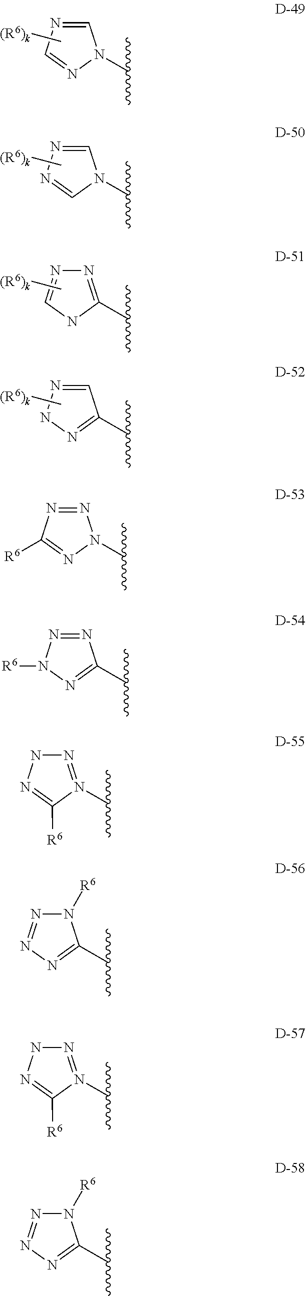

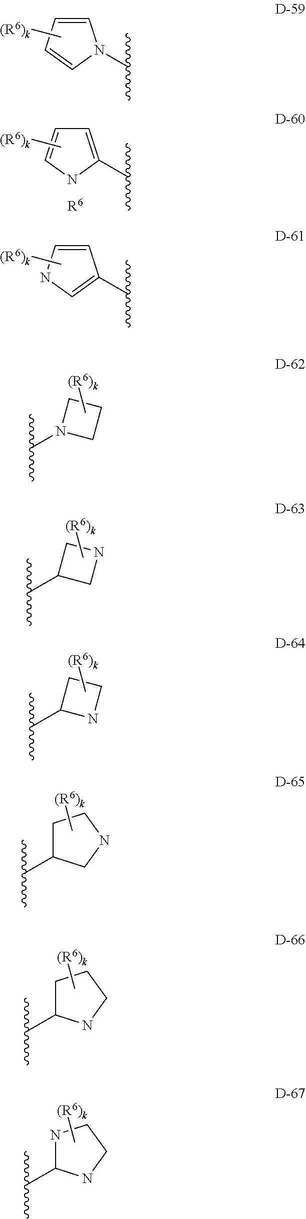

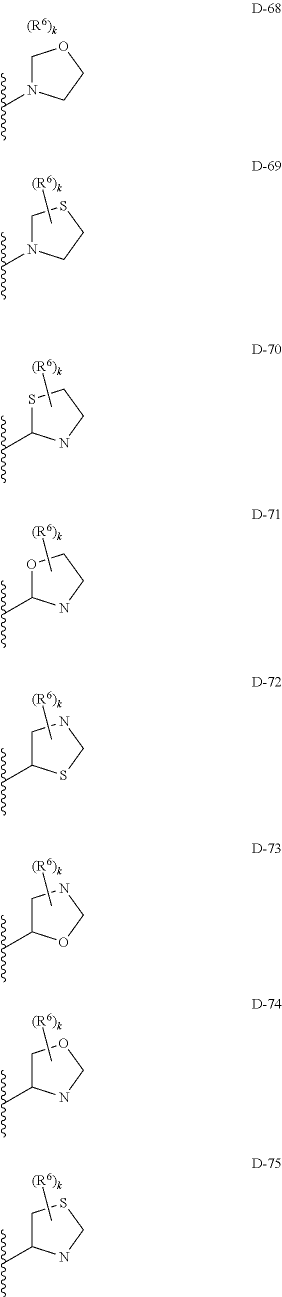

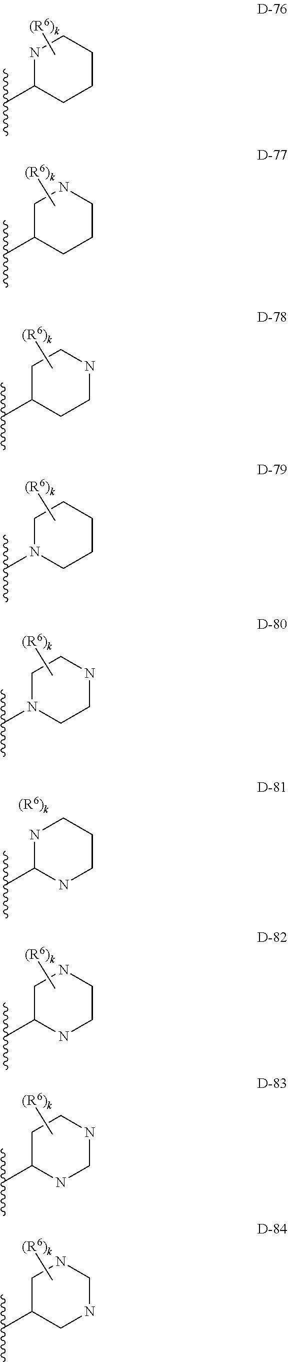

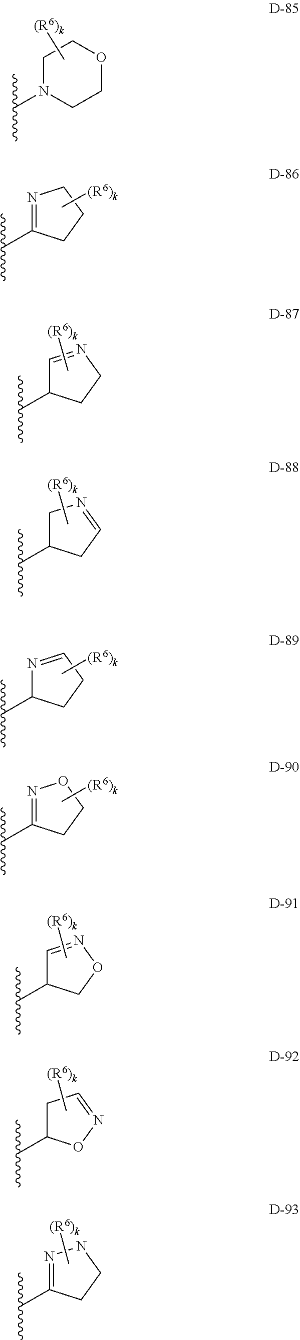

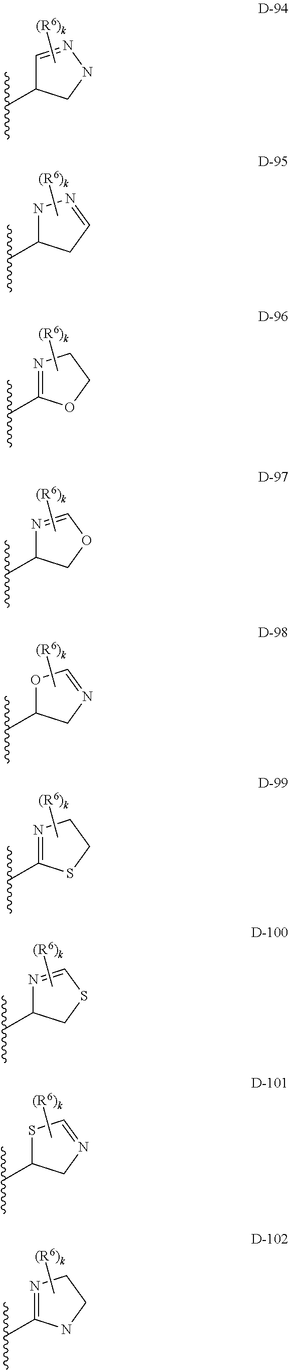

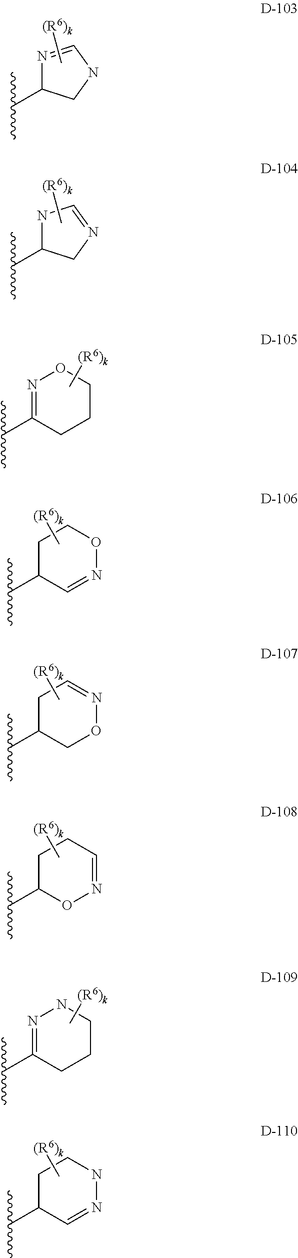

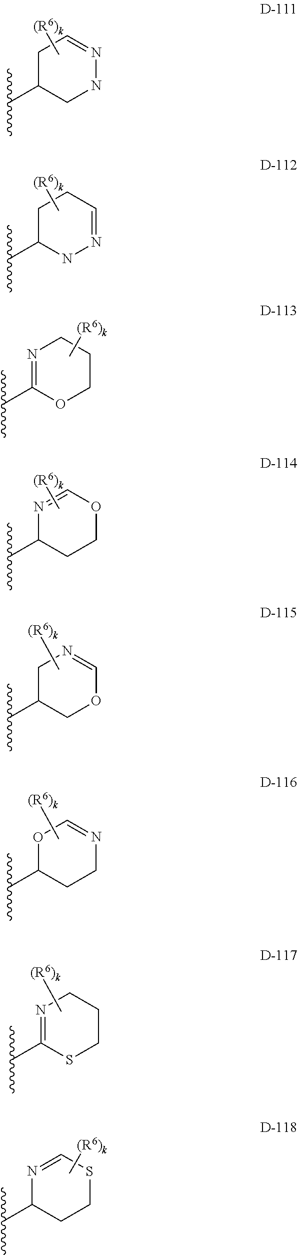

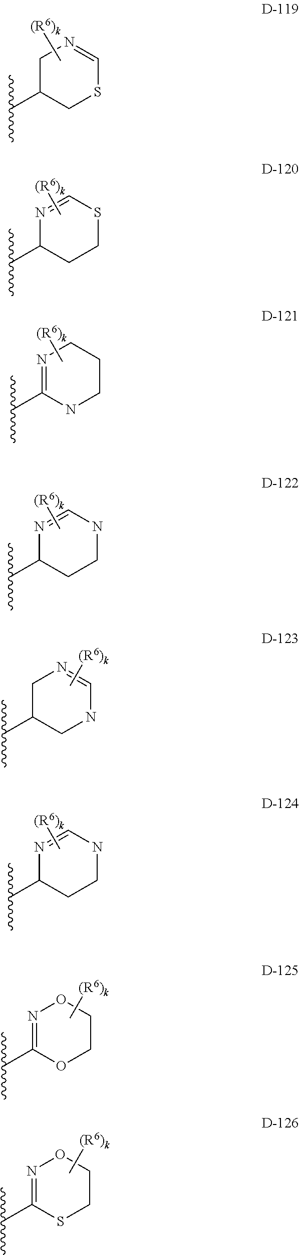

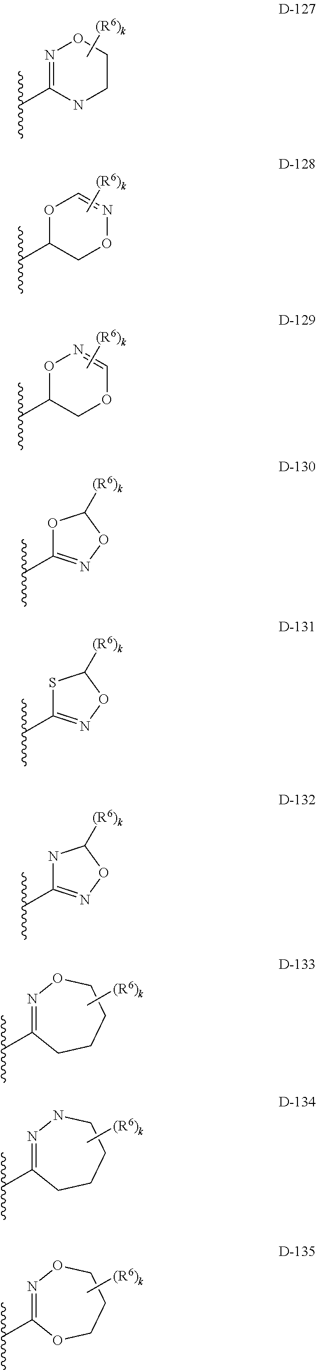

wherein the zigzag line denotes the bond to the thiazole ring of formula (I) and k is an integer selected from 0, 1, 2, 3, 4, 5 or 6 and R⁶ is defined dependently of the nature of R² and dependently of the integer of k as described herein above.

Especially more preferred are substituted 3-pyridyl thiazole compounds of the general formula (I) of the present invention, wherein
R² is selected from the group consisting of partially or fully halogenated C₁-C₄-haloalkyl.
A² is the nitrogen containing 4-, 5-, or 6-membered heterocyclic or hetero-aromatic ring system is selected from any of the following ring systems (as defined above) D-1, D-2, D-3, D-5, D-7, D-9, D-19, D-21, D-22, D-26, D-27, D-28, D-29, D-30, D-31, D-32, D-37, D-45, D-49, D-50, D-51, D-90, D-93, D-96, D-99, D-102, D-113, D-117, D-121, D-125, D-126, D-127, D-130, D-131, D-132, D-135, and wherein each
   R⁶ is independently selected from the group consisting of hydrogen, halogen, cyano, nitro, C₁-C₆-alkyl, C₃-C₆-cycloalkyl, wherein the carbon atoms of the aforementioned aliphatic and cycloaliphatic radicals may optionally be partially or fully halogenated and/or may carry 1 or 2 radicals R¹⁵, which are selected independently from one another,
      OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶;
      phenyl, pyridyl or phenoxy, each optionally substituted with one or more, e.g. 1, 2, 3, 4, or 5 substituents selected independently from R¹⁸;
      or
      two R⁶ present on one ring carbon atom may together form ═O, ═S, ═NR^{17a}; and wherein
   k is 0, 1, 2 or 3.

Especially preferred are substituted 3-pyridyl thiazole compounds of the general formula (I) of the present invention, wherein
R¹ is hydrogen;
R² is selected from the group consisting of CHF₂, CF₃, CHCl₂, CCl₃ and C₂-C₄ haloalkyl.
A² is a nitrogen containing 4-, 5-, or 6-membered heterocyclic or hetero-aromatic ring system selected from any of the following ring systems:
   D-1, D-2, D-3, D-5, D-7, D-9, D-19, D-21, D-22, D-26, D-27, D-28, D-29, D-30, D-31, D-32, D-37, D-45, D-49, D-50, D-51, D-90, D-93, D-96, D-99, D-102, D-113, D-117, D-121, D-125, D-126, D-127, D-130, D-131, D-132, D-135 as defined in claim 11, and wherein each
   R⁶ is independently selected from the group consisting of hydrogen, halogen, cyano, nitro, C₁-C₆-alkyl, C₃-C₆-cycloalkyl, wherein the carbon atoms of the aforementioned aliphatic and cycloaliphatic radicals may optionally be partially or fully halogenated and/or may carry 1 or 2 radicals R¹⁵, which are selected independently from one another,
      OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶;
      phenyl, pyridyl or phenoxy, each optionally substituted with one or more, e.g. 1, 2, 3, 4, or 5 substituents selected independently from R¹⁸; or two R⁶ present on one ring carbon atom may together form ═O, ═S, ═NR^{17a};
      and wherein
   k is 0, 1, 2 or 3.

Especially more preferred are substituted 3-pyridyl thiazole compounds of the general formula (I) of the present invention, wherein
R² is fluoro, chloro or bromo;
A² is the nitrogen containing 4-, 5-, or 6-membered heterocyclic or hetero-aromatic ring system is selected from any of the following ring systems (as defined above) D-1, D-2, D-3, D-5, D-7, D-9, D-19, D-21, D-22, D-26, D-27, D-28, D-29, D-30, D-31, D-32, D-37, D-45, D-49, D-50, D-51, D-90, D-93, D-96, D-99, D-102, D-113, D-117, D-121, D-125, D-126, D-127, D-130, D-131, D-132, D-135, and wherein each
   R⁶ is independently from one another selected from the group consisting of, halogen, cyano, C₁-C₁₆-alkyl, C₃-C₆-cycloalkyl, C₂-C₆-alkenyl, C₂-C₆-alkinyl, and wherein the carbon atoms of the aforementioned aliphatic and cycloaliphatic radicals may optionally be further substituted independently from one another with one or more R¹⁵,
      OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶;
      or two R⁶ present on one ring carbon atom may together form ═O, ═CR¹³R¹⁴; ═S; ═NR^{17a}, ═NOR¹⁶; ═NNR^{17a};
      and wherein
   k is 1, 2 or 3.

Especially more preferred are substituted 3-pyridyl thiazole compounds of the general formula (I) of the present invention, wherein
R² is fluoro, chloro or bromo;
A² is the nitrogen containing 4-, 5-, or 6-membered heterocyclic or hetero-aromatic ring system is selected from any of the following ring systems (as defined above) D-1, D-2, D-3, D-5, D-7, D-9, D-19, D-21, D-22, D-26, D-27, D-28, D-29, D-30, D-31, D-32, D-37, D-45, D-49, D-50, D-51, D-90, D-93, D-96, D-99, D-102, D-113, D-117, D-121, D-125, D-126, D-127, D-130, D-131, D-132, D-135, and wherein each
   R⁶ is independently from one another selected from the group consisting of, halogen, cyano, C₁-C₁₆-alkyl, C₃-C₆-cycloalkyl, C₂-C₆-alkenyl, C₂-C₆-alkinyl, and wherein the carbon atoms of the aforementioned aliphatic and cycloaliphatic radicals may optionally be further substituted independently from one another with one or more R¹⁵,
      OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶;
      phenyl, optionally substituted with one or more, e.g. 1, 2, or 3, substituents selected independently from R¹⁰; or a 5- or 6-membered saturated, partly saturated or unsaturated aromatic heterocyclic ring comprising 1, 2, or 3 heteroatoms selected from oxygen, nitrogen and/or sulfur, optionally substituted with one or more, e.g. 1, 2, or 3 substituents selected independently from R¹⁰, and wherein the nitrogen and/or the sulfur atom(s) of the heterocyclic ring may optionally be oxidized,
      or two R⁶ present on one ring carbon atom may together form ═O, ═CR¹³R¹⁴; ═S; ═NR^{17a}, ═NOR¹⁶; ═NNR^{17a};
      and wherein
   k is 2 or 3.

Further examples of especially preferred compounds of formula I for the purposes of the present invention are given herein below, without imposing any limitation to this invention.

A preferred embodiment of the present invention are compounds of the following 12 formulae II-1 to II-12, wherein the variables R² and R⁶ have one of the general or preferred meanings given above.

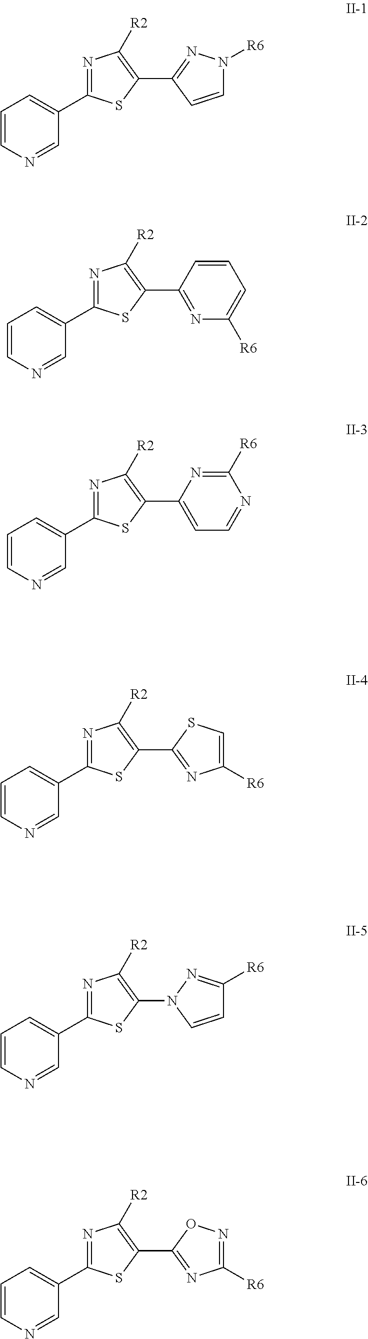

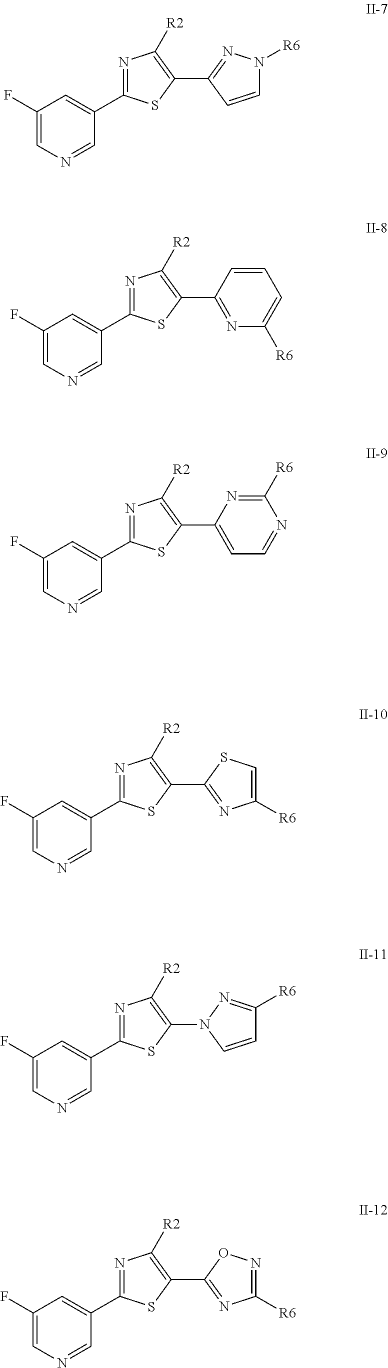

Specific examples of especially preferred compounds for the purposes of the present invention are represented by the formulae II-1 to II-12 in combination with table C.I (part I) hereinafter defining R² and R⁶.

The meaning of both substituents, R² and R⁶, are defined by their combination as given in one row of table C.I (part I), thereby showing individual preferred compounds compiled in table C.I. (part I) herein below.

TABLE C.I
(part I):

| Compound | R² | R⁶ |
|---|---|---|
| C.I.1 | Cl | H |
| C.I.2 | F | H |
| C.I.3 | Br | H |
| C.I.4 | CF₃ | H |
| C.I.5 | CF₂H | H |
| C.I.6 | CF₂CF₃ | H |
| C.I.7 | Cl | CF₃ |
| C.I.8 | F | CF₃ |
| C.I.9 | Br | CF₃ |
| C.I.10 | CF₃ | CF₃ |
| C.I.11 | CF₂H | CF₃ |
| C.I.12 | CF₂CF₃ | CF₃ |
| C.I.13 | Cl | Cl |
| C.I.14 | F | Cl |
| C.I.15 | Br | Cl |
| C.I.16 | CF₃ | Cl |
| C.I.17 | CF₂H | Cl |
| C.I.18 | CF₂CF₃ | Cl |
| C.I.19 | Cl | Br |
| C.I.20 | F | Br |
| C.I.21 | Br | Br |
| C.I.22 | CF₃ | Br |
| C.I.23 | CF₂H | Br |
| C.I.24 | CF₂CF₃ | Br |
| C.I.25 | Cl | CF₂H |
| C.I.26 | F | CF₂H |
| C.I.27 | Br | CF₂H |
| C.I.28 | CF₃ | CF₂H |
| C.I.29 | CF₂H | CF₂H |
| C.I.30 | CF₂CF₃ | CF₂H |
| C.I.31 | Cl | CF₂H |
| C.I.32 | F | CF₂H |
| C.I.33 | Br | CF₂H |
| C.I.34 | CF₃ | CF₂H |
| C.I.35 | CF₂H | CF₂H |
| C.I.36 | CF₂CF₃ | CF₂H |
| C.I.37 | Cl | CH₃ |
| C.I.38 | F | CH₃ |
| C.I.39 | Br | CH₃ |
| C.I.40 | CF₃ | CH₃ |
| C.I.41 | CF₂H | CH₃ |
| C.I.42 | CF₂CF₃ | CH₃ |
| C.I.43 | Cl | CH₂CH₃ |
| C.I.44 | F | CH₂CH₃ |
| C.I.45 | Br | CH₂CH₃ |
| C.I.46 | CF₃ | CH₂CH₃ |
| C.I.47 | CF₂H | CH₂CH₃ |
| C.I.48 | CF₂CF₃ | CH₂CH₃ |
| C.I.49 | Cl | C(CH₃)₃ |
| C.I.50 | F | C(CH₃)₃ |
| C.I.51 | Br | C(CH₃)₃ |
| C.I.52 | CF₃ | C(CH₃)₃ |
| C.I.53 | CF₂H | C(CH₃)₃ |
| C.I.54 | CF₂CF₃ | C(CH₃)₃ |
| C.I.55 | Cl | CF(CF₃)₂ |
| C.I.56 | F | CF(CF₃)₂ |
| C.I.57 | Br | CF(CF₃)₂ |
| C.I.58 | CF₃ | CF(CF₃)₂ |
| C.I.59 | CF₂H | CF(CF₃)₂ |
| C.I.60 | CF₂CF₃ | CF(CF₃)₂ |
| C.I.61 | Cl | F |
| C.I.62 | F | F |
| C.I.63 | Br | F |
| C.I.64 | CF₃ | F |
| C.I.65 | CF₂H | F |
| C.I.66 | CF₂CF₃ | F |
| C.I.67 | Cl | N(CH₃)₂ |
| C.I.68 | F | N(CH₃)₂ |
| C.I.69 | Br | N(CH₃)₂ |
| C.I.70 | CF₃ | N(CH₃)₂ |
| C.I.71 | CF₂H | N(CH₃)₂ |
| C.I.72 | CF₂CF₃ | N(CH₃)₂ |
| C.I.73 | Cl | OCF₃ |
| C.I.74 | F | OCF₃ |
| C.I.75 | Br | OCF₃ |
| C.I.76 | CF₃ | OCF₃ |
| C.I.77 | CF₂H | OCF₃ |
| C.I.78 | CF₂CF₃ | OCF₃ |
| C.I.79 | Cl | 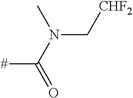 |
| C.I.80 | F | 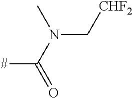 |
| C.I.81 | Br | 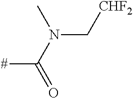 |
| C.I.82 | CF₃ | 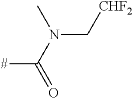 |
| C.I.83 | CF₂H | 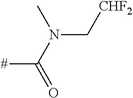 |
| C.I.84 | CF₂CF₃ | 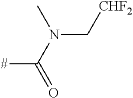 |
| C.I.85 | Cl | 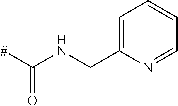 |
| C.I.86 | F | 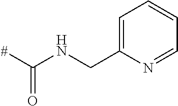 |
| C.I.87 | Br | 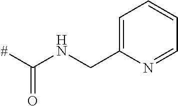 |
| C.I.88 | CF₃ | 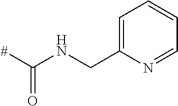 |
| C.I.89 | CF₂H | 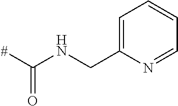 |
| C.I.90 | CF₂CF₃ | 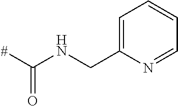 |
| C.I.91 | Cl | 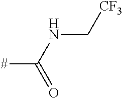 |
| C.I.92 | F | 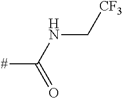 |
| C.I.93 | Br | 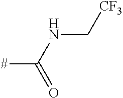 |
| C.I.94 | CF₃ | 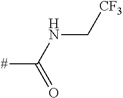 |
| C.I.95 | CF₂H | 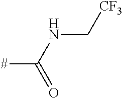 |
| C.I.96 | CF₂CF₃ | 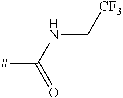 |
| C.I.97 | Cl | 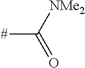 |
| C.I.98 | F | 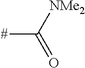 |
| C.I.99 | Br | 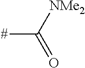 |
| C.I.100 | CF₃ | 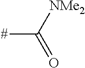 |
| C.I.101 | CF₂H | 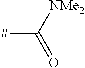 |
| C.I.102 | CF₂CF₃ | 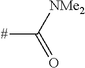 |
| C.I.103 | Cl | 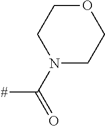 |
| C.I.104 | F | 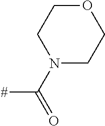 |
| C.I.105 | Br | 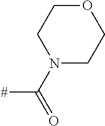 |
| C.I.106 | CF₃ | 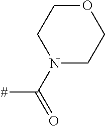 |
| C.I.107 | CF₂H | 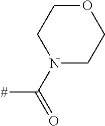 |
| C.I.108 | CF₂CF₃ | 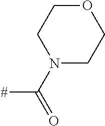 |
| C.I.109 | Cl | 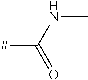 |
| C.I.110 | F | 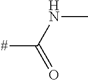 |
| C.I.111 | Br | 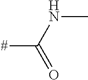 |
| C.I.112 | CF₃ | 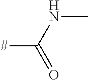 |
| C.I.113 | CF₂H | 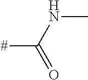 |
| C.I.114 | CF₂CF₃ | 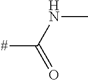 |
| C.I.115 | Cl | 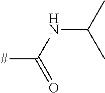 |
| C.I.116 | F | 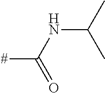 |
| C.I.117 | Br | 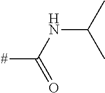 |
| C.I.118 | CF₃ | 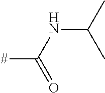 |
| C.I.119 | CF₂H | 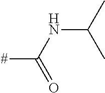 |
| C.I.120 | CF₂CF₃ | 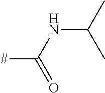 |
| C.I.121 | Cl | 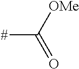 |
| C.I.122 | F | 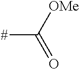 |
| C.I.123 | Br | 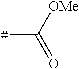 |
| C.I.124 | CF₃ | 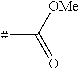 |
| C.I.125 | CF₂H | 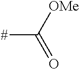 |
| C.I.126 | CF₂CF₃ | 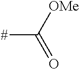 |
| C.I.127 | Cl | 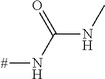 |
| C.I.128 | F | 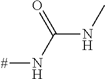 |
| C.I.129 | Br | 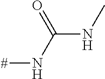 |
| C.I.130 | CF₃ | 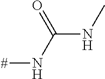 |
| C.I.131 | CF₂H | 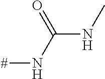 |
| C.I.132 | CF₂CF₃ | 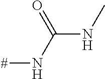 |
| C.I.133 | Cl | 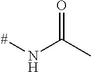 |
| C.I.134 | F | 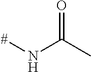 |
| C.I.135 | Br | 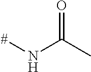 |
| C.I.136 | CF₃ | 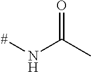 |
| C.I.137 | CF₂H | 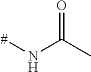 |
| C.I.138 | CF₂CF₃ | 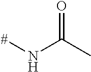 |
| C.I.139 | Cl | 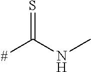 |
| C.I.140 | F | 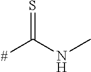 |
| C.I.141 | Br | 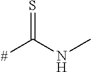 |
| C.I.142 | CF₃ | 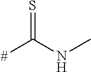 |
| C.I.143 | CF₂H | 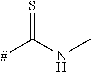 |
| C.I.144 | CF₂CF₃ | 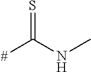 |
| C.I.145 | Cl | 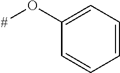 |
| C.I.146 | F | 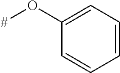 |
| C.I.147 | Br | 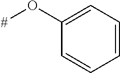 |
| C.I.148 | CF₃ | 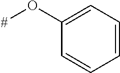 |
| C.I.149 | CF₂H | 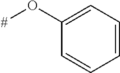 |
| C.I.150 | CF₂CF₃ | 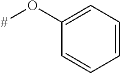 |
| C.I.151 | Cl | 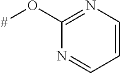 |
| C.I.152 | F | 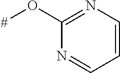 |
| C.I.153 | Br | 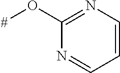 |
| C.I.154 | CF₃ | 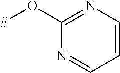 |
| C.I.155 | CF₂H | 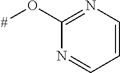 |
| C.I.156 | CF₂CF₃ | 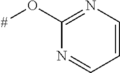 |

wherein # of R⁶ denotes the bond in the molecule

For example, synthesis example S.1 herein further below shows the preparation of 4-methyl-5-(1H-pyrazol-3-yl)-2-(3-pyridyl)thiazole, alternatively termed 3-[5-(1H-Pyrazol-3-yl)-4-trifluoromethyl-thiazol-2-yl]-pyridine:

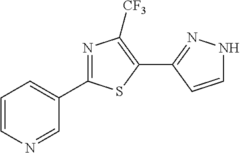 [SMILES: FC(F)(F)c1nc(-c2cccnc2)sc1-c1cc[nH]n1]

which corresponds to compound example C.I.4 of table C.I. with formula II-1.

Another preferred embodiment of the present invention are compounds of the following 12 formulae II-13 to II-24, wherein the variables R² and R⁶ have one of the general or preferred meanings given above.

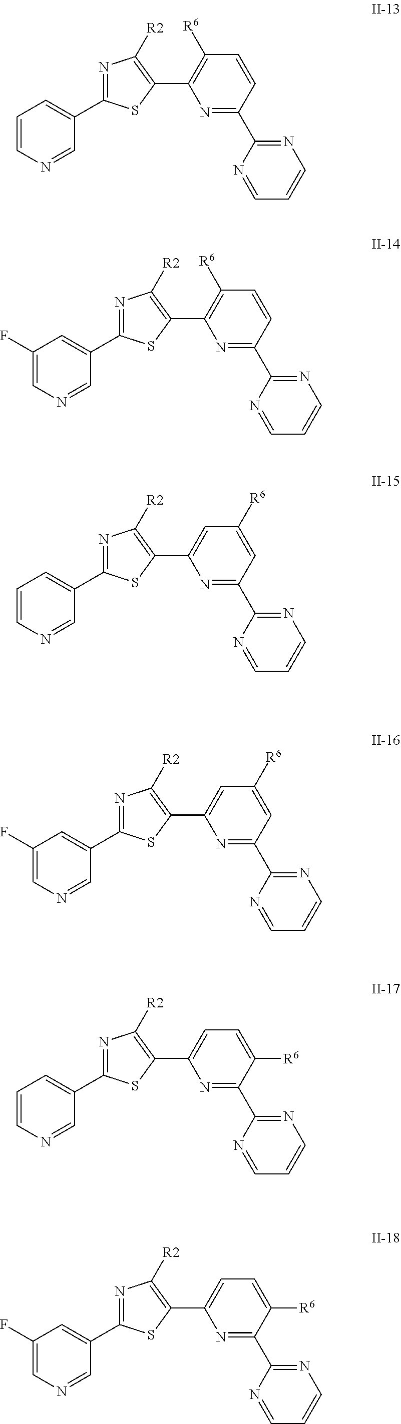

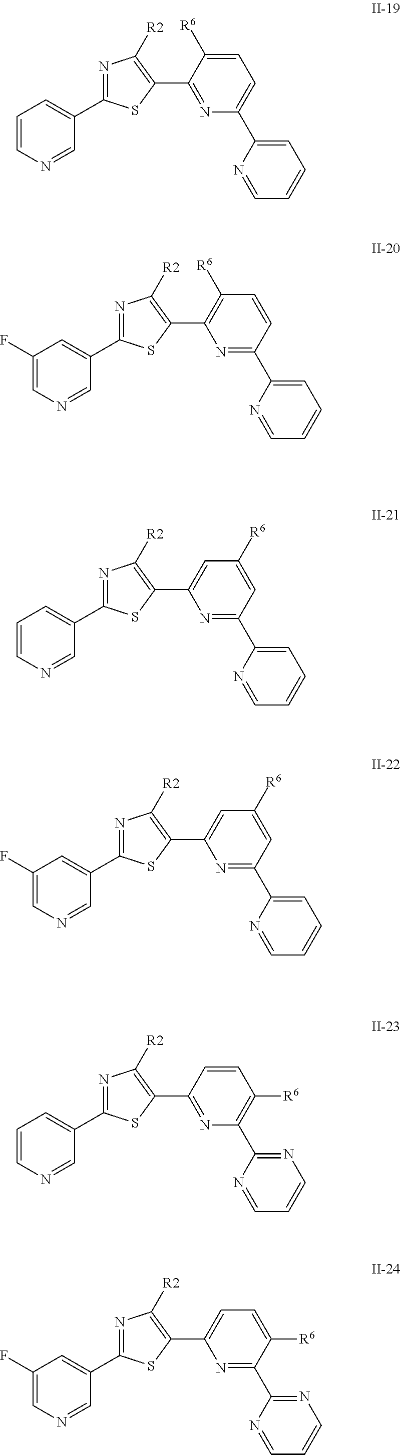

Specific examples of especially preferred compounds for the purposes of the present invention are represented by the formulae II-13 to II-24 in combination with the table C.I (part II) hereinafter defining R² and R⁶.

The meaning of both substituents, R² and R⁶, are defined by their combination as given in one row of table C.I (part II), thereby showing individual preferred compounds compiled in the of table C.I. (part II)

TABLE C.I.
(part II)

| Compound | R² | R⁶ |
|---|---|---|
| C.I.157 | Cl | F |
| C.I.158 | F | F |
| C.I.159 | Br | F |
| C.I.160 | CF₃ | F |
| C.I.161 | CF₂H | F |
| C.I.162 | CF₂CF₃ | F |
| C.I.163 | Cl | CF₃ |
| C.I.164 | F | CF₃ |
| C.I.165 | Br | CF₃ |
| C.I.166 | CF₃ | CF₃ |
| C.I.167 | CF₂H | CF₃ |
| C.I.168 | CF₂CF₃ | CF₃ |
| C.I.169 | Cl | Cl |
| C.I.170 | F | Cl |
| C.I.171 | Br | Cl |
| C.I.172 | CF₃ | Cl |
| C.I.173 | CF₂H | Cl |
| C.I.174 | CF₂CF₃ | Cl |
| C.I.175 | Cl | Br |
| C.I.176 | F | Br |
| C.I.177 | Br | Br |
| C.I.178 | CF₃ | Br |
| C.I.179 | CF₂H | Br |
| C.I.180 | CF₂CF₃ | Br |
| C.I.181 | Cl | CF₂H |
| C.I.182 | F | CF₂H |
| C.I.183 | Br | CF₂H |
| C.I.184 | CF₃ | CF₂H |
| C.I.185 | CF₂H | CF₂H |
| C.I.186 | CF₂CF₃ | CF₂H |
| C.I.187 | Cl | CF₂H |
| C.I.188 | F | CF₂H |
| C.I.189 | Br | CF₂H |
| C.I.190 | CF₃ | CF₂H |
| C.I.191 | CF₂H | CF₂H |
| C.I.192 | CF₂CF₃ | CF₂H |
| C.I.193 | Cl | CH₃ |
| C.I.194 | F | CH₃ |
| C.I.195 | Br | CH₃ |
| C.I.196 | CF₃ | CH₃ |
| C.I.197 | CF₂H | CH₃ |
| C.I.198 | CF₂CF₃ | CH₃ |
| C.I.199 | Cl | CH₂CH₃ |
| C.I.200 | F | CH₂CH₃ |
| C.I.201 | Br | CH₂CH₃ |
| C.I.202 | CF₃ | CH₂CH₃ |
| C.I.203 | CF₂H | CH₂CH₃ |
| C.I.204 | CF₂CF₃ | CH₂CH₃ |
| C.I.205 | Cl | C(CH₃)₃ |
| C.I.206 | F | C(CH₃)₃ |
| C.I.207 | Br | C(CH₃)₃ |
| C.I.208 | CF₃ | C(CH₃)₃ |
| C.I.209 | CF₂H | C(CH₃)₃ |
| C.I.210 | CF₂CF₃ | C(CH₃)₃ |
| C.I.211 | Cl | CF(CF₃)₂ |
| C.I.212 | F | CF(CF₃)₂ |
| C.I.213 | Br | CF(CF₃)₂ |
| C.I.214 | CF₃ | CF(CF₃)₂ |
| C.I.215 | CF₂H | CF(CF₃)₂ |
| C.I.216 | CF₂CF₃ | CF(CF₃)₂ |
| C.I.217 | Cl | OCH₃ |
| C.I.218 | F | OCH₃ |
| C.I.219 | Br | OCH₃ |
| C.I.220 | CF₃ | OCH₃ |
| C.I.221 | CF₂H | OCH₃ |
| C.I.222 | CF₂CF₃ | OCH₃ |
| C.I.223 | Cl | N(CH₃)₂ |
| C.I.224 | F | N(CH₃)₂ |
| C.I.225 | Br | N(CH₃)₂ |
| C.I.226 | CF₃ | N(CH₃)₂ |
| C.I.227 | CF₂H | N(CH₃)₂ |
| C.I.228 | CF₂CF₃ | N(CH₃)₂ |
| C.I.229 | Cl | OCF₃ |
| C.I.230 | F | OCF₃ |
| C.I.231 | Br | OCF₃ |
| C.I.232 | CF₃ | OCF₃ |
| C.I.233 | CF₂H | OCF₃ |
| C.I.234 | CF₂CF₃ | OCF₃ |
| C.I.235 | Cl | 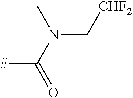 |
| C.I.236 | F | 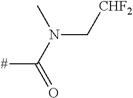 |
| C.I.237 | Br | 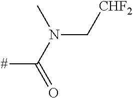 |
| C.I.238 | CF₃ | 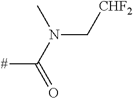 |
| C.I.239 | CF₂H | 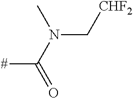 |
| C.I.240 | CF₂CF₃ | 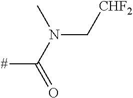 |
| C.I.241 | Cl | 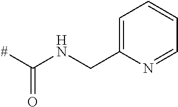 |
| C.I.242 | F | 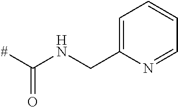 |
| C.I.243 | Br | 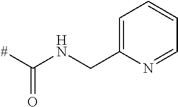 |
| C.I.244 | CF₃ | 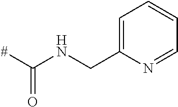 |
| C.I.245 | CF₂H | 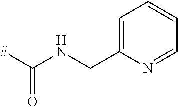 |
| C.I.246 | CF₂CF₃ | 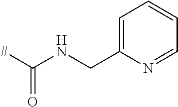 |
| C.I.247 | Cl | 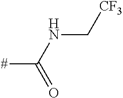 |
| C.I.248 | F | 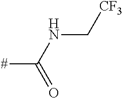 |
| C.I.249 | Br | 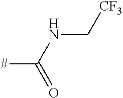 |
| C.I.250 | CF₃ | 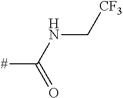 |
| C.I.251 | CF₂H | 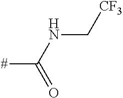 |
| C.I.252 | CF₂CF₃ | 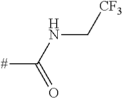 |
| C.I.253 | Cl | 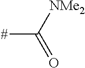 |
| C.I.254 | F | 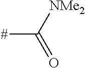 |
| C.I.255 | Br | 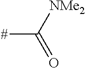 |
| C.I.256 | CF₃ | 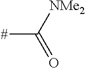 |
| C.I.257 | CF₂H | 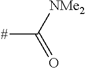 |
| C.I.258 | CF₂CF₃ | 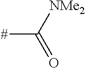 |
| C.I.259 | Cl | 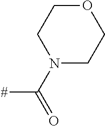 |
| C.I.260 | F | 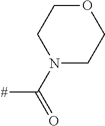 |
| C.I.261 | Br | 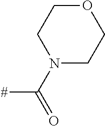 |
| C.I.262 | CF₃ | 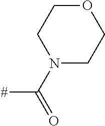 |
| C.I.263 | CF₂H | 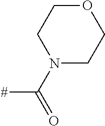 |
| C.I.264 | CF₂CF₃ | 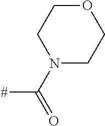 |
| C.I.265 | Cl | 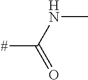 |
| C.I.266 | F | 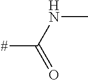 |
| C.I.267 | Br | 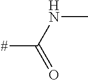 |
| C.I.268 | CF₃ | 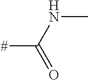 |
| C.I.269 | CF₂H | 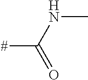 |
| C.I.270 | CF₂CF₃ | 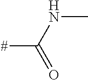 |
| C.I.271 | Cl | 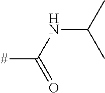 |
| C.I.272 | F | 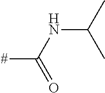 |
| C.I.273 | Br | 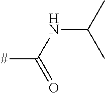 |
| C.I.274 | CF₃ | 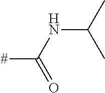 |
| C.I.275 | CF₂H | 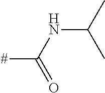 |
| C.I.276 | CF₂CF₃ | 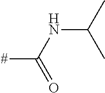 |
| C.I.277 | Cl | 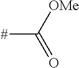 |
| C.I.278 | F | 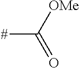 |
| C.I.279 | Br | 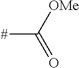 |
| C.I.280 | CF₃ | 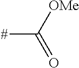 |
| C.I.281 | CF₂H | 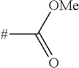 |
| C.I.282 | CF₂CF₃ | 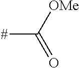 |
| C.I.283 | Cl | 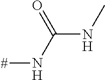 |
| C.I.284 | F | 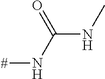 |
| C.I.285 | Br | 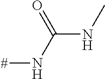 |
| C.I.286 | CF₃ | 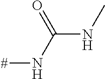 |
| C.I.287 | CF₂H | 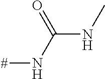 |
| C.I.288 | CF₂CF₃ | 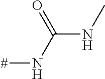 |
| C.I.289 | Cl | 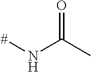 |
| C.I.290 | F | 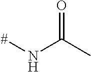 |
| C.I.291 | Br | 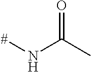 |
| C.I.292 | CF₃ | 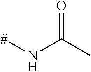 |
| C.I.293 | CF₂H | 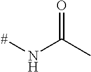 |
| C.I.294 | CF₂CF₃ | 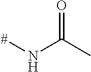 |
| C.I.295 | Cl | 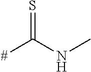 |
| C.I.296 | F | 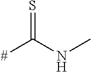 |
| C.I.297 | Br | 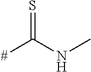 |
| C.I.298 | CF₃ | 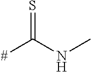 |
| C.I.299 | CF₂H | 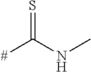 |
| C.I.300 | CF₂CF₃ | 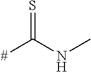 |

wherein # of R⁶ denotes the bond in the molecule

Moreover, the meanings mentioned for those individual variables in the tables are per se, independently of the combination in which they are mentioned, a particularly preferred embodiment of the substituents in question.

### Preparation Methods

Compound of formula (I) according to the present invention can be prepared according to the following synthesis routes, e.g. according the preparation methods and preparation schemes as described below.

Compounds of formula (I) according to the present invention can generally be prepared by standard methods of organic chemistry e.g. by the preparation methods and preparation schemes as described below. If not otherwise specified for defined conditions, the definitions of A, R¹, R², and R⁶ of the molecular structures given in the schemes are as defined above. Room temperature means a temperature range between about 20 and 25° C.

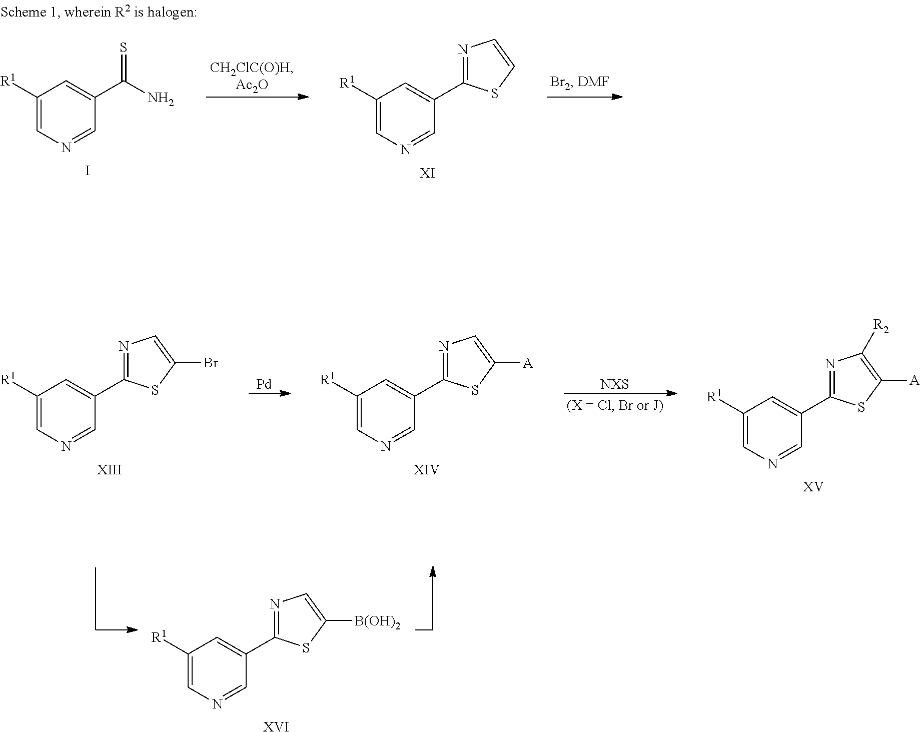

Thioamides of formula I can be reacted with acetic anhydride and chloroacetaldehyde as described in WO 2010006713 to provide 2-substituted thiazoles of formula XII (Scheme 1). Bromination of such compounds using for example bromine in a solvent such as dimethylformamide in analogy to J. Med. Chem., 2006, 49 (5) 1730, can then provide access to derivatives of formula XIII. The introduction of heterocycles to provide compounds of formula XIV can be carried out using for example copper(I) iodide and a pyrazole compound (N-linked pyrazoles WO 2010006713), using for example pyridine boronic acids in the presence of a Pd catalyst (see Bioorg. Med. Chem. Let., 2010, 20 (9) 2828-2831), pyridazine boronic acids in the presence of a Pd catalyst (see WO 2007041632), pyrimidine boronic acids in the presence of a Pd catalyst (see WO 2009149858).

Alternatively, compounds of formula XIII can be converted to the corresponding boronic acid (XVI) upon treatment with for example lithium diisopropylamide and trimethoxyborane (see WO 2008017688. Boronic acids of formula XVI can then undergo Suzuki couplings under Pd catalysis to afford derivatives of formula XIV (see for example WO 2008017688 and WO 2007071436 for similar examples).

4-halothiazoles of formula XV can be prepared from the compounds of formula XIV by treatment with an appropriate halogenating reagent such as N-halosuccinimides (NXS) as for example N-chlorosuccinimide (NCS) or N-bromosuccinimide (NBS) see WO 20100129497).

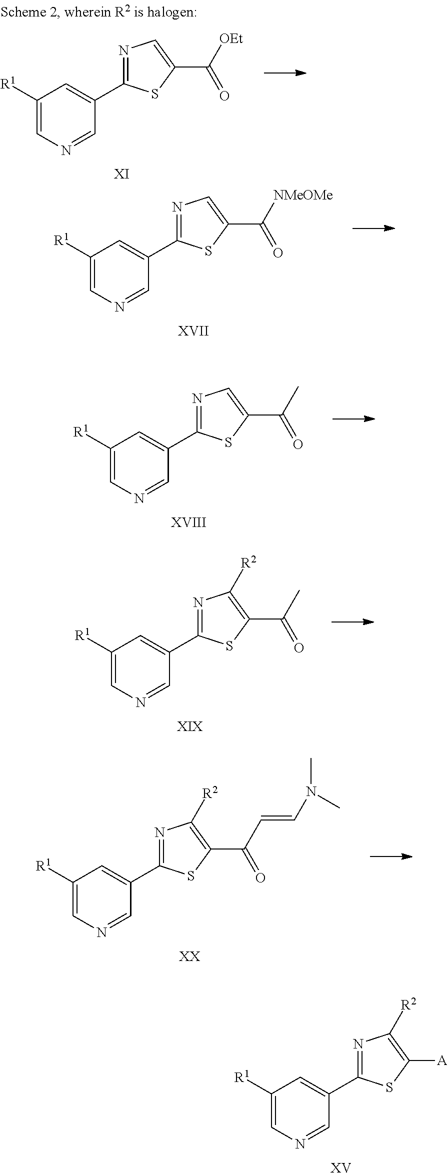

An alternative strategy for the synthesis of compounds of formula XV is depicted in Scheme 2. Ester derivatives of formula XI can be converted to the corresponding acetyl compounds of formula XVIII via the Weinreb amide (XVII) in analogy to WO 201006713. Halogenation of compounds with formula XVIII can then be carried out using standard halogenating reagents such as for example N-halosucccinimides (in analogy to WO 20100129497). Treatment of XIX with DMF acetal in analogy to WO 201006713 affords XX. Compounds of formula XX can be converted to heterocycles such as for example C-linked pyrazoles upon treatment with hydrazines. or for example pyrimidines upon treatment with amidines (see WO 2010006713).

Certain compounds which fall under the formula XV may be accessed via condensation chemistry starting from the carboxylic acid derivative IV (Scheme 3). See for example WO 2010006713.

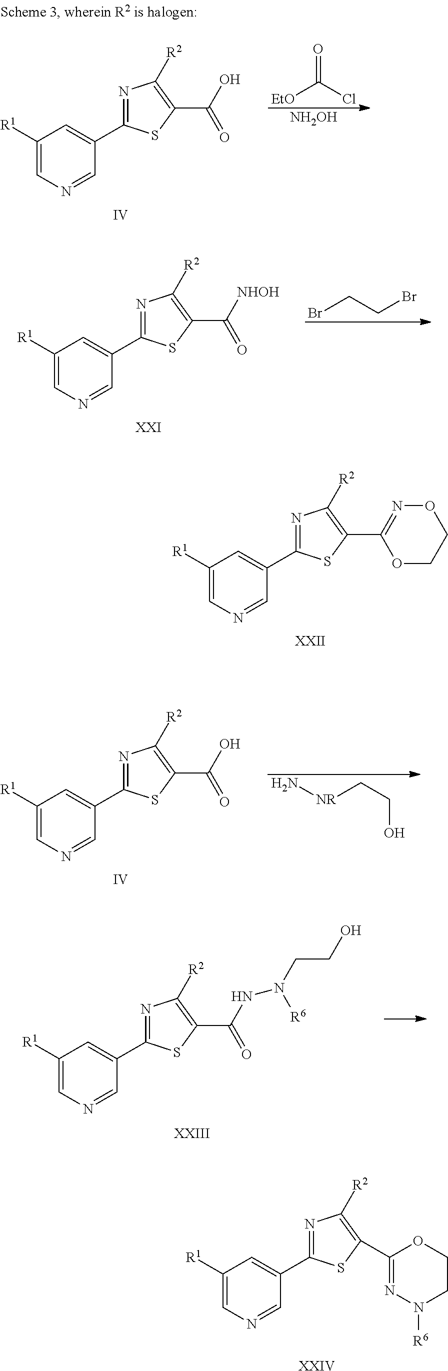

For the case where R²=haloalkyl, compounds of formula XXVIII may be prepared according to Scheme 4. The preparation of thiazoles with formula III can be achieved starting from thioamides of formula I via reaction with 2-chloro-3-oxo-butyric acid ethyl ester derivatives (II) in analogy to WO 2010012947

Ester derivatives of formula III can be converted to the corresponding vinylogous amide derivatives XXVII in a 3-step sequence in analogy to WO 201006713. Compounds of formula XXVII can be converted to heterocycles such as for example C-linked pyrazoles upon treatment with hydrazines. or for example pyrimidines upon treatment with amidines (see WO 2010006713).

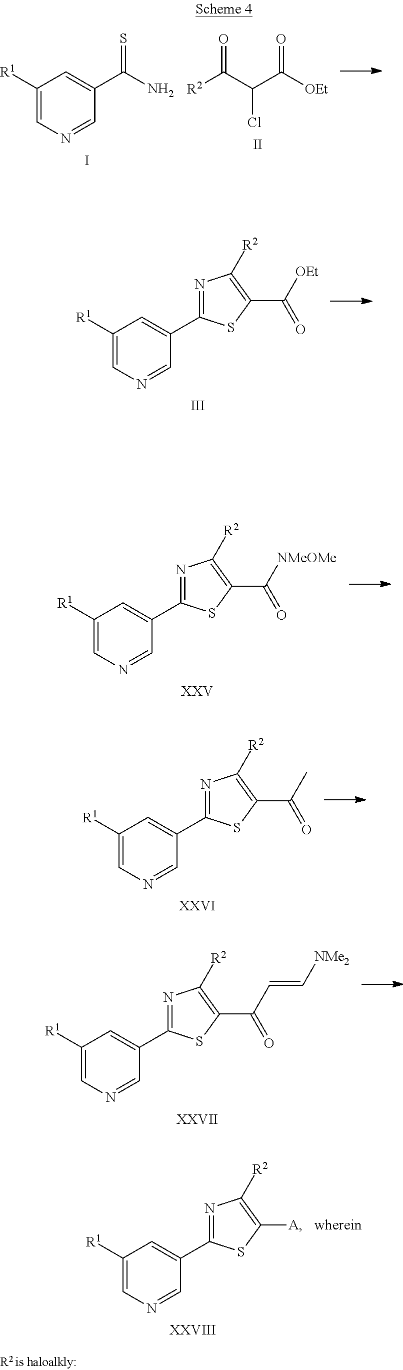

An alternative approach to the synthesis of compounds of formula XXVIII is depicted in Scheme 5. Derivatives of formula XXIX may be prepared from the esters III via decarboxylation according to several methods such as for example silver(I) carbonate and acetic acid in a solvent such as dimethylsulfoxide (Org. Lett., 2009, 11(24), 5710), for example aqueous phosphoric acid (Bioorg. Med. Chem. 2007,15(20), 6574) or for example hydrochloric acid (J. Chem. Soc., Perkin Trans. 1, 1983, (2), 341). Compounds of formula XXIX may then be brominated using for example bromine (J. Med. Chem., 2006, 49 (5) 1730) to provide XXX. Compounds of formula XXX may then themselves undergo transition-metal catalysed coupling reactions (see Scheme 1 and associated text) or first be converted to the corresponding boronic acid derivatives of formula XXXI which then undergo such transition-metal mediated coupling reactions (see Scheme 1 and associated text).

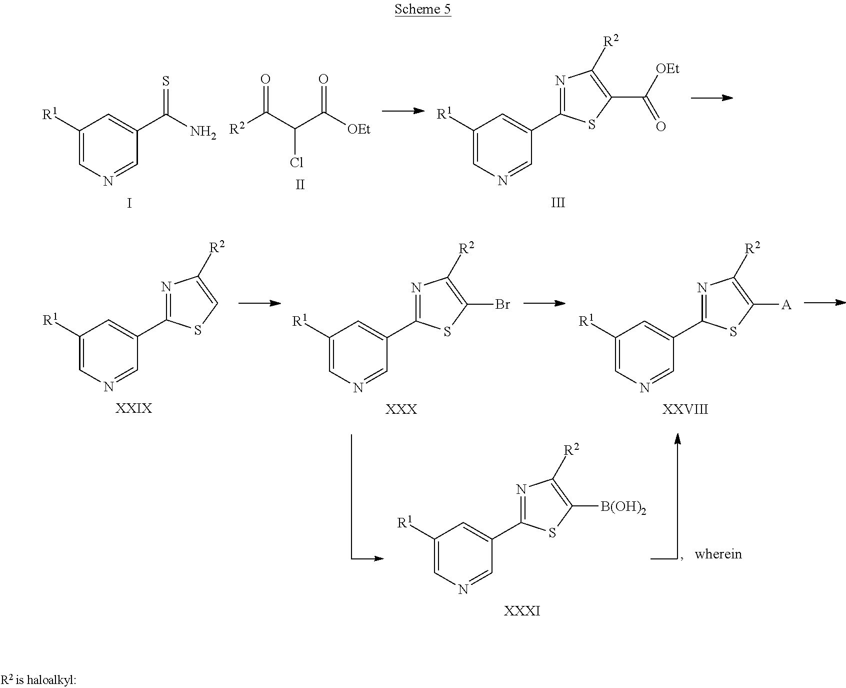

Certain compounds which fall under the formula XV may be accessed via condensation chemistry starting from ethyl ester derivative IV (Scheme 6) see, for example WO 2010006713.

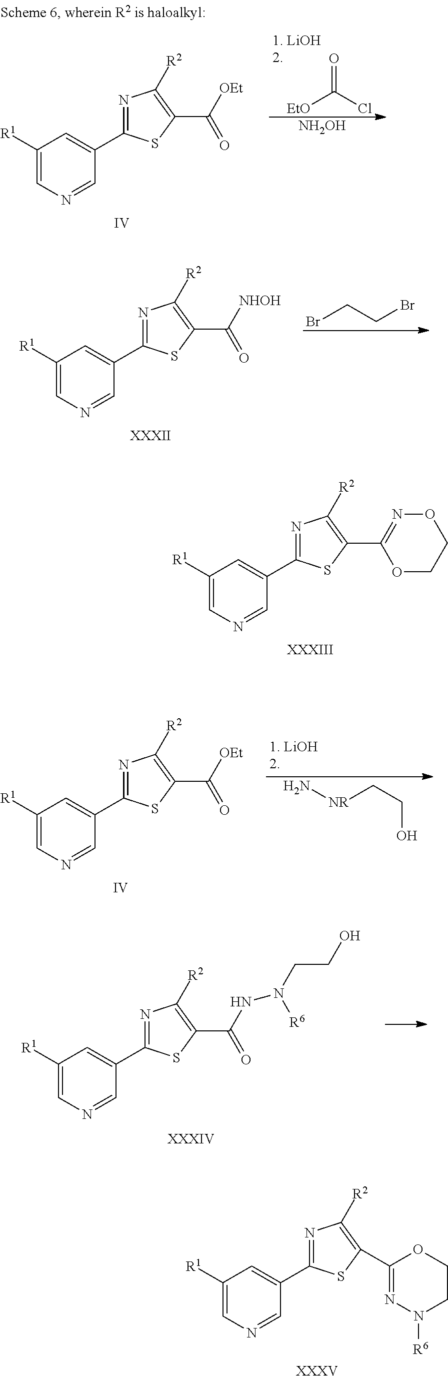

If individual compounds cannot be prepared via the above-described routes, they can be prepared by derivatization of other compounds (I) or by customary modifications of the synthesis routes described.

For example, in individual cases, certain compounds of formula (I) can advantageously be prepared from other compounds of formula (I) by derivatization, e.g. by ester hydrolysis, amidation, esterification, ether cleavage, olefination, reduction, oxidation and the like, or by customary modifications of the synthesis routes described.

The reaction mixtures are worked up in the customary manner, for example by mixing with water, separating the phases, and, if appropriate, purifying the crude products by chromatography, for example on alumina or silica gel. Some of the intermediates and end products may be obtained in the form of colorless or pale brown viscous oils, which are freed or purified from volatile components under reduced pressure and at moderately elevated temperature. If the intermediates and end products are obtained as solids, they may be purified by recrystallization, trituration or digestion.

### Pests

The term “invertebrate pest” as used herein encompasses animal populations, such as arthropode pests, including insects and arachnids, as well as nematodes, which may attack plants thereby causing substantial damage to the plants attacked, as well as ectoparasites which may infest animals, in particular warm blooded animals such as e.g. mammals or birds, or other higher animals such as reptiles, amphibians or fish, thereby causing substantial damage to the animals infested.

The compounds of the formula I, and their salts are in particular suitable for efficiently controlling arthropodal pests such as arachnids, myriapedes and insects as well as nematodes.

The compounds of the formula I are especially suitable for efficiently combating the following pests:

Insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Cheimatobia brumata, Choristoneura fumiferana, Choristoneura occidentalis, Cirphis unipuncta, Cydia pomonella, Dendrolimus Diaphania nitidalis, Diatraea grandiosella, Earias insulana, Elasmopalpus lignosellus, Eupoecilia ambiguella, Evetria bouliana, Feltia subterranea, Gallena mellonella, Grapholitha funebrana, Grapholitha molesta, Heliothis armigera, Heliothis virescens, Heliothis zea, Hellula undalis, Hibernia defoliaria, Hyphantna cunea, Hyponomeuta Keifena lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, TortriX viridana, Trichoplusia ni and Zeiraphera canadensis;

beetles (Coleoptera), for example Agrilus sinuatus, Agriotes lineatus, Agriotes obscurus, Amphimallus solstitialis, Anisandrus dispar, Anthonomus grandis, Anthonomus pomorum, Aphthona euphoridae, Athous haemorrhoidalis, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Cetonia aurata, Ceuthorrhynchus assimilis, Ceuthorrhynchus nap, Chaetocnema Conoderus vespertinus, Crioceris asparagi, Ctenicera ssp., Diabrotica longicornis, Diabrotica semipunctata, Diabrotica 12-punctata Diabrotica speciosa, Diabrotica virgifera, Epilachna varivestis, EpitriX hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Otiorrhynchus sulcatus, Otiorrhynchus ovatus, Phaedon cochleanae, Phyllobius pyri, Phyllotreta chrysocephala, Phyllophaga sp., Phyllopertha horticola, Phyllotreta nemorum, Phyllotreta striolata, Popillia japonica, Sitona lineatus and Sitophilus granaria;

flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora Ceratitis capitata, Chiysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza Lucilia caprin, Lucilia cuprina, Lucilia sericata, Lycoria pectoralis, Mansonia titillanus, Mayetiola destructor, Musca autumnalis, Musca domesfica, Muscina stabulans, Oestrus ovis, Opomyza forum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga spp., Simulium vittatum, Stomoxys calcitrans, Tabanus bovinus, Tabanus atratus, Tabanus lineola, and Tabanus similis, Tipula oleracea, and Tipula paludosa;

trips (Thysanoptera), e.g. Dichromothrips corbetti, Dichromothrips ssp., Frankliniella fusca, Frankliniella occidentalis, Frankliniella tritici, Scirtothrips citri, Thrips oryzae, Thrips palmi and Thrips tabaci,

termites (Isoptera), e.g. Calotermes flavicollis, Leucotermes flavipes, Heterotermes aureus, Reticulitermes flavipes, Reticulitermes virginicus, Reticulitermes lucifugus, Reticulitermes santonensis, Reticulitermes grassei, Termes natalensis, and Coptotermes formosanus;

cockroaches (Blattaria—Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,

bugs, aphids, leafhoppers, whiteflies, scale insects, cicadas (Hemiptera), e.g. Acrosternum hilare, Blissus leucopterus, Cyrtopeltis notatus, Dysdercus angulatus, Dysdercus intermedius, Eurygaster integriceps, Euschistus impictiventris, Leptoglossus phyllopus, Lygus lineolaris, Lygus pratensis, Nezara vindula, Piesma quadrata, Solubea insularis, Thyanta perditor, Acyrthosiphon onobrychis, Adelges laricis, Aphidula nasturtii, Aphis fabae, Aphis fortesi, Aphis pomi, Aphis gossypii, Aphis grossulanae, Aphis schneiden, Aphis spiraecola, Aphis sambuci, Acyrthosiphon pisum, Aulacorthum solani, Bemisia argentifolf, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis gyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzus persicae, Myzus ascalonicus, Myzus ceras Myzus varians, Nasonovia nbis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mak, Schizaphis graminum, Schizoneura lanuginosa, Sitobion avenae, Tnaleurodes vaporariorum, Toxoptera aurantii and, Viteus vitifolii, Cimex lectularius, Cimex hemipterus, Reduvius senilis, Triatoma spp., and Arilus critatus;

ants, bees, wasps, sawflies (Hymenoptera), e.g. Athalia rosae, Atta cephalotes, Atta capiguara, Atta cephalotes, Atta laevigata, Atta robusta, Atta sexdens, Atta texana, Crematogaster spp., Hoplocampa minuta, Hoplocampa testudinea, Lasius niger, Monomorium pharaonis, Solenopsis geminata, Solenopsis invicta, Solenopsis richteri, Soienopsis xyloni, Pogonomyrmex barbatus, Pogonomyrmex californicus, Pheidole megacephala, Dasymutilla occidentalis, Bombus spp., Vespula squamosa, Paravespula vulgaris, Paravespula pennsylvanica, Paravespula germanica, Dolichovespula maculata, Vespa crabro, Polistes rubiginosa, Camponotus floridanus, and Linepithema humile;

crickets, grasshoppers, locusts (Orthoptera), e.g. Acheta domestica, Gryllotalpa gryllotalpa, Locusta migratoria, Melanoplus bivittatus, Melanoplus femurrubrum, Melanoplus mexicanus, Melanoplus sanguinipes, Melanoplus spretus, Nomadacris septemfasciata, Schistocerca americana, Schistocerca gregaria, Dociostaurus maroccanus, Tachycines asynamorus, Oedaleus senegalensis, Zonozerus variegatus, Hieroglyphus daganensis, Kraussaria angulifera, Calliptamus italicus, Chortoicetes terminifera, and Locustana pardalina;

arachnids (Arachnoidea), such as acarians (Acarina), e.g. of the families Argasidae, Ixodidae and Sarcoptidae, such as Amblyomma americanum, Amblyomma variegatum, Ambryomma maculatum, Argas persicus, Boophllus annulatus, Boophllus decoloratus, Boophllus microplus, Dermacentor silvarum, Dermacentor andersoni, Dermacentor variabilis, Hyalomma truncatum, Ixodes ricinus, Ixodes rubicundus, Ixodes scapularis, Ixodes holocyclus, Ixodes pacificus, Ornithodorus moubata, Ornithodorus hermsi, Ornithodorus turicata, Ornithonyssus bacoti, Otobius megnini Dermanyssus gallinae, Psoroptes ovis, Rhipicephalus sanguineus, Rhipicephalus appendiculatus, Rhipicephalus evertsi, Sarcoptes scabiei, and Eriophyidae spp. such as Aculus schlechtendali Phyllocoptrata oleivora and Eriophyes sheldoni, Tarsonemidae spp. such as Phytonemus pallidus and Polyphagotarsonemus latus; Tenuipalpidae spp. such as Brevipalpus phoenicis Tetranychidae spp. such as Tetranychus cinnabarinus, Tetranychus kanzawai, Tetranychus pacificus, Tetranychus telarius and Tetranychus urticae, Panonychus ulmi, Panonychus citri, and Oligonychus pratensis; Araneida, e.g. Latrodectus mactans, and Loxosceles reclusa;

fleas (Siphonaptera), e.g. Ctenocephalides Ctenocephalides canis, Xenopsylla cheopis, Pulex irritans, Tunga penetrans, and Nosopsyllus fasciatus,

silverfish, firebrat (Thysanura), e.g. Lepisma saccharin and Thermobia domestica,

centipedes (Chilopoda), e.g. Scutigera coleoptrata,

millipedes (Diplopoda), e.g. Narceus spp.,

Earwigs (Dermaptera), e.g. forficula auricularia,

lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.

Collembola (springtails), e.g. Onychiurus ssp.

They are also suitable for controlling Nematodes: plant parasitic nematodes such as root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursapheienchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

Compounds of the formula I are particularly useful for controlling insects, preferably sucking or piercing insects such as insects from the genera Thysanoptera, Diptera and Hemiptera, in particular the following species:

Thysanoptera: Frankliniella fusca, Frankliniella occidentalis, Frankliniella tritici, Scirtothrips citri, Thrips oryzae, Thrips palmi and Thrips tabaci,

Diptera, e.g. Aedes aegypti, Aedes albopictus, Aedes vexans, Anastrepha ludens, Anopheles maculipennis, Anopheles crucians, Anopheles albimanus, Anopheles gambiae, Anopheles freeborn, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia caprina, Lucllia cuprina, Lucilia sericata, Lycoria pectoralis, Mansonia titillanus, Mayetiola destructor, Musca autumnalis, Musca domestica, Muscina stabulans, Oestrus ovis, Opomyza forum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga spp., Simulium vittatum, Stomoxys calcitrans, Tabanus bovinus, Tabanus atratus, Tabanus lineola, and Tabanus similis, Tipula oleracea, and Tipula paludosa;

Hemiptera, in particular aphids: Acyrthosiphon onobrychis, Adelges laricis, Aphidula nasturtii, Aphis fabae, Aphis forbesi, Aphis porni, Aphis gossypii, Aphis grossulanae, Aphis schneideri, Aphis spiraecola, Aphis sambuci, Acyrthosiphon pisum, Aulacorthum solani, Brachycaudus cardui, Brachycaudus helichlysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis gyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mall, Schizaphis graminum, Schizoneura lanuginosa, Sitobion avenae, Tnaleurodes vaporariorum, Toxoptera aurantiland, and Viteus vitifolii.

Compounds of the formula I are particularly useful for controlling insects of the orders Hemiptera and Thysanoptera.

### Formulations

The invention also relates to agrochemical compositions comprising an auxiliary and at least one compound I according to the invention.

An agrochemical composition comprises a pesticidally effective amount of a compound I. The term “effective amount” denotes an amount of the composition or of the compounds I, which is sufficient for controlling harmful pests on cultivated plants or in the protection of materials and which does not result in a substantial damage to the treated plants. Such an amount can vary in a broad range and is dependent on various factors, such as the animal pests species to be controlled, the treated cultivated plant or material, the climatic conditions and the specific compound I used.

The compounds I, their N-oxides and salts can be converted into customary types of agrochemical compositions, e. g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Mono-graph No. 2, 6th Ed. May 2008, CropLife International.

The compositions are prepared in a known manner, such as described by Mollet and Grube-mann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e.g. ethanol, propanol, butanol, benzylalcohol, cyclo [symbol] hexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, lime-stone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. ce-real meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyhnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Exam-pies of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides.

Examples of polymeric surfactants are home- or copolymers of vinylpyrrolidone, vinyl-alcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the compound I on the target. Examples are surfactants, mineral or vegetable oils, and other auxilaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), anorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanofer-rate) and organic colorants (e.g. alizarin-, azo- and phthalocyanine colorants).

Suitable tackifiers or binders are polyvinylpyrrolidons, polyvinylacetates, polyvinyl alcohofs, pol-yacrylates, biological or synthetic waxes, and cellulose ethers.

Examples for composition types and their preparation are:
i) Water-soluble concentrates (SL, LS)
   10-60 wt % of a compound I according to the invention and 5-15 wt % wetting agent (e.g. alcohol alkoxylates) are dissolved in water and/or in a water-soluble solvent (e.g. alcohols) ad 100 wt %. The active substance dissolves upon dilution with water.
ii) Dispersible concentrates (DC)
   5-25 wt % of a compound I according to the invention and 1-10 wt % dispersant (e. g. poly-vinylpyrrolidone) are dissolved in organic solvent (e.g. cyclohexanone) ad 100 wt %. Dilution with water gives a dispersion.
iii) Emulsifiable concentrates (EC)
   15-70 wt % of a compound I according to the invention and 5-10 wt % emulsifiers (e.g. calcium dodecylbenzenesulfonate and castor oil ethoxylate) are dissolved in water-insoluble organic solvent (e.g. aromatic hydrocarbon) ad 100 wt %. Dilution with water gives an emulsion.
iv) Emulsions (EW, EO, ES)
   5-40 wt % of a compound I according to the invention and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into water ad 100 wt % by means of an emulsifying machine and made into a homogeneous emulsion. Dilution with water gives an emulsion.
v) Suspensions (SC, OD, FS)
   In an agitated ball mill, 20-60 wt % of a compound I according to the invention are comminuted with addition of 2-10 wt % dispersants and wetting agents (e.g. sodium lignosulfonate and alcohol ethoxylate), 0.1-2 wt % thickener (e.g. xanthan gum) and water ad 100 wt % to give a fine active substance suspension. Dilution with water gives a stable suspension of the active substance. For FS type composition up to 40 wt % binder (e.g. polyvinylalcohol) is added.
vi) Water-dispersible granules and water-soluble granules (WG, SG)
   50-80 wt % of a compound I according to the invention are ground finely with addition of dispersants and wetting agents (e.g. sodium lignosulfonate and alcohol ethoxylate) ad 100 wt % and prepared as water-dispersible or water-soluble granules by means of technical appliances (e. g. extrusion, spray tower, fluidized bed). Dilution with water gives a stable dispersion or solution of the active substance.
vii) Water-dispersible powders and water-soluble powders (WP, SP, WS)
   50-80 wt % of a compound I according to the invention are ground in a rotor-stator mill with addition of 1-5 wt % dispersants (e.g. sodium lignosulfonate), 1-3 wt % wetting agents (e.g. alcohol ethoxylate) and solid carrier (e.g. silica gel) ad 100 wt %. Dilution with water gives a stable dispersion or solution of the active substance.
viii) Gel (GW, GF)
   In an agitated ball mill, 5-25 wt % of a compound I according to the invention are comminuted with addition of 3-10 wt % dispersants (e.g. sodium lignosulfonate), 1-5 wt % thickener (e.g. carboxymethylcellulose) and water ad 100 wt % to give a fine suspension of the active substance. Dilution with water gives a stable suspension of the active substance.
iv) Microemulsion (ME)
   5-20 wt % of a compound I according to the invention are added to 5-30 wt % organic solvent blend (e.g. fatty acid dimethylamide and cyclohexanone), 10-25 wt % surfactant blend (e.g. alkohol ethoxylate and arylphenol ethoxylate), and water ad 100%. This mixture is stirred for 1 h to produce spontaneously a thermodynamically stable microemulsion.
iv) Microcapsules (CS)
   An oil phase comprising 5-50 wt % of a compound I according to the invention, 0-40 wt % water insoluble organic solvent (e.g. aromatic hydrocarbon), 2-15 wt % acrylic monomers (e.g. methylmethacrylate, methacrylic acid and a di- or triacry-late) are dispersed into an aqueous solution of a protective colloid (e.g. polyvinyl alcohol). Radical polymerization initiated by a radical initiator results in the formation of poly(meth)acrylate microcapsules. Alternatively, an oil phase comprising 5-50 wt % of a compound I according to the invention, 0-40 wt % water insoluble organic solvent (e.g. aromatic hydrocarbon), and an isocyanate monomer (e.g. diphenylmethene-4,4′-diisocyanatae) are dispersed into an aqueous solution of a protective colloid (e.g. polyvinyl alcohol). The addition of a polyamine (e.g. hexa-methylenediamine) results in the formation of a polyurea microcapsules. The monomers amount to 1-10 wt %. The wt % relate to the total CS composition.
ix) Dustable powders (DP, DS)
   1-10 wt % of a compound I according to the invention are ground finely and mixed intimately with solid carrier (e.g. finely divided kaolin) ad 100 wt %.
x) Granules (GR, FG)
   0.5-30 wt % of a compound I according to the invention is ground finely and associated with solid carrier (e.g. silicate) ad 100 wt %. Granulation is achieved by extrusion, spray-drying or the fluidized bed.
xi) Ultra-low volume liquids (UL)
   1-50 wt % of a compound I according to the invention are dissolved in organic solvent (e.g. aromatic hydrocarbon) ad 100 wt %.

The compositions types i) to xi) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, and in particular between 0.5 and 75%, by weight of active substance. The active substances are employed in a purity of from 90% to 100%, preferably from 95% to 100% (according to NMR spectrum).

Solutions for seed treatment (LS), Suspo-emulsions (SE), flowable concentrates (FS), powders for dry treatment (DS), water-dispersible powders for slurry treatment (WS), water-soluble pow-ders (SS), emulsions (ES), emulsifiable concentrates (EC) and gels (GF) are usually employed for the purposes of treatment of plant propagation materials, particularly seeds. The compositions in question give, after two-to-tenfold dilution, active substance concentrations of from 0.01 to 60% by weight, preferably from 0.1 to 40% by weight, in the ready-to-use preparations. Application can be carried out before or during sowing. Methods for applying compound I and compositions thereof, respectively, on to plant propagation material, especially seeds include dressing, coating, pelleting, dusting, soaking and in-furrow application methods of the propagation material. Preferably, compound I or the compositions thereof, respectively, are applied on to the plant propagation material by a method such that germination is not induced, e. g. by seed dressing, pelleting, coating and dusting.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, and in particular from 0.1 to 0.75 kg per ha.

In treatment of plant propagation materials such as seeds, e. g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seeds) are generally required. When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e.g. herbicides, insecticides, fungicides, growth regulators, safeners) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

The user applies the composition according to the invention usually from a predosage device, a knapsack sprayer, a spray tank, a spray plane, or an irrigation system. Usually, the agrochemi-cal composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank and further auxiliaries may be added, if appropriate.

In a further embodiment, either individual components of the composition according to the invention or partially premixed components, e. g. components comprising compounds I, may be mixed by the user in a spray tank and further auxiliaries and additives may be added, if appropriate.

In a further embodiment, either individual components of the composition according to the invention or partially premixed components, e. g. components comprising compounds I, can be applied jointly (e.g. after tank mix) or consecutively.

### Mixtures

According to one embodiment of the present invention, individual components of the composition according to the invention such as parts of a kit or parts of a binary or ternary mixture may be mixed by the user himself in a spray tank and further auxiliaries may be added, if appropriate.

In a further embodiment, either individual components of the composition according to the invention or partially premixed components, e. g. components comprising compounds I and/or active substances from the groups M.1 to M.UN.X or F.I to F.XII, may be mixed by the user in a spray tank and further auxiliaries and additives may be added, if appropriate.

In a further embodiment, either individual components of the composition according to the invention or partially premixed components, e. g. components comprising compounds I and/or active substances from the groups M.1 to M.UN.X or F.I to F.XII, can be applied jointly (e.g. after tank mix) or consecutively.

The following list M of pesticides, grouped according the Mode of Action Classification of the Insecticide Resistance Action Committee (IRAC), together with which the compounds according to the invention can be used and with which potential synergistic effects might be produced, is intended to illustrate the possible combinations, but not to impose any limitation:

M.1 Acetylcholine esterase (AChE) inhibitors from the class of

M.1A carbamates, for example aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb and triazamate; or from the class of

M.1B organophosphates, for example acephate, azamethiphos, azinphos-ethyl, azinphosmethyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothio-phosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon and vamidothion;

M.2. GABA-gated chloride channel antagonists such as:

M.2A cyclodiene organochlorine compounds, as for example endosulfan or chlordane; or

M.2B fiproles (phenylpyrazoles), as for example ethiprole, fipronil, flufiprole, pyrafluprole and pyriprole;

M.3 Sodium channel modulators from the class of

M.3A pyrethroids, for example acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, meperfluthrin, metofluthrin, permethrin, phenothrin, prallethrin, profluthrin, pyrethrin (pyrethrum), resmethrin, silafluofen, tefluthrin, tetramethylfluthrin, tetramethrin, tralomethrin and transfluthrin; or

M.3B sodium channel modulators such as DDT or methoxychlor;

M.4 Nicotinic acetylcholine receptor agonists (nAChR) from the class of

M.4A neonicotinoids, for example acteamiprid, chlothianidin, dinotefuran, imidacloprid, nitenpyram, thiacloprid and thiamethoxam; or M.4B nicotine.

M.5 Nicotinic acetylcholine receptor allosteric activators from the class of spinosyns, for example spinosad or spinetoram;

M.6 Chloride channel activators from the class of avermectins and milbemycins, for example abamectin, emamectin benzoate, ivermectin, lepimectin or milbemectin;

M.7 Juvenile hormone mimics, such as

M.7A juvenile hormone analogues as hydroprene, kinoprene and methoprene; or others as M.7B fenoxycarb or M.7C pyriproxyfen;

M.8 miscellaneous non-specific (multi-site) inhibitors, for example

M.8A alkyl halides as methyl bromide and other alkyl halides, or

M.8B chloropicrin, or M.8C sulfuryl fluoride, or M.8D borax, or M.8E tartar emetic;

M.9 Selective homopteran feeding blockers, for example

M.9B pymetrozine, or M.9C flonicamid;

M.10 Mite growth inhibitors, for example

M.10A clofentezine, hexythiazox and diflovidazin, or M.10B etoxazole;

M.11 Microbial disruptors of insect midgut membranes, for example bacillus thuringiensis or bacillus sphaericus and the insecticdal proteins they produce such as bacillus thuringiensis subsp. israelensis, bacillus sphaericus, bacillus thuringiensis subsp. aizawai, bacillus thuringiensis subsp. kurstaki and bacillus thuringiensis subsp. tenebrionis, or the Bt crop proteins: Cry1Ab, Cry1Ac, Cry1Fa, Cry2Ab, mCry3A, Cry3Ab, Cry3Bb and Cry34/35Ab1;

M.12 Inhibitors of mitochondrial ATP synthase, for example

M.12A diafenthiuron, or

M.12B organotin miticides such as azocyclotin, cyhexatin or fenbutatin oxide, or M.12C propargite, or M.12D tetradifon;

M.13 Uncouplers of oxidative phosphorylation via disruption of the proton gradient, for example chlorfenapyr, DNOC or sulfluramid;

M.14 Nicotinic acetylcholine receptor (nAChR) channel blockers, for example nereistoxin analogues as bensultap, cartap hydrochloride, thiocyclam or thiosultap sodium;

M.15 Inhibitors of the chitin biosynthesis type 0, such as benzoylureas as for example bistrifluron, chlorfluazuron, diflubenzuron, flucycloxuron, flufenoxuron, hexaflumuron, lufenuron, novaluron, noviflumuron, teflubenzuron or triflumuron;

M.16 Inhibitors of the chitin biosynthesis type 1, as for example buprofezin;

M.17 Moulting disruptors, Dipteran, as for example cyromazine;

M.18 Ecdyson receptor agonists such as diacylhydrazines, for example methoxyfenozide, tebufenozide, halofenozide, fufenozide or chromafenozide;

M.19 Octopamin receptor agonists, as for example amitraz;

M.20 Mitochondrial complex III electron transport inhibitors, for example

M.20A hydramethylnon, or M.20B acequinocyl, or M.20C fluacrypyrim;

M.21 Mitochondrial complex I electron transport inhibitors, for example

M.21A METI acaricides and insecticides such as fenazaquin, fenpyroximate, pyrimidifen, pyridaben, tebufenpyrad or tolfenpyrad, or M.21B rotenone;

M.22 Voltage-dependent sodium channel blockers, for example

M.22A indoxacarb, or M.22B metaflumizone;

M.23 Inhibitors of the of acetyl CoA carboxylase, such as Tetronic and Tetramic acid derivatives, for example spirodiclofen, spiromesifen or spirotetramat;

M.24 Mitochondrial complex IV electron transport inhibitors, for example

M.24A phosphine such as aluminium phosphide, calcium phosphide, phosphine or zinc phosphide, or M.24B cyanide.

M.25 Mitochondrial complex II electron transport inhibitors, such as betaketonitrile derivatives, for example cyenopyrafen or cyflumetofen;

M.28 Ryanodine receptor-modulators from the class of diamides, as for example flubendiamide, chloranthraniliprole (Rynaxypyr®), cyanthraniliprole (Cyazypyr®), or the phthalamide compounds
M.28.1: (R)-3-Chlor-N1-{2-methyl-4-[1,2,2,2-tetrafluor-1-(trifluormethyl)ethyl]phenyl}-N2-(1-methyl-2-methylsulfonylethyl)phthalamid and
M.28.2: (S)-3-Chlor-N1-{2-methyl-4-[1,2,2,2-tetrafluor-1-(trifluormethyl)ethyl]phenyl}-N2-(1-methyl-2-methylsulfonylethyl)phthalamid, or the compound
M.28.3: 3-bromo-N-{2-bromo-4-chloro-6-[(1-cyclopropylethyl)carbamoyl]phenyl}-1-(3-chlorpyridin-2-yl)-1H-pyrazole-5-carboxamide, or the compound
M.28.4: methyl-2-[3,5-dibromo-2-({[3-bromo-1-(3-chlorpyridin-2-yl)-1H-pyrazol-5-yl]carbonyl}amino)benzoyl]-1,2-dimethylhydrazinecarboxylate;

M.UN.X insecticidal active compounds of unknown or uncertain mode of action, as for example azadirachtin, amidoflumet, benzoximate, bifenazate, bromopropylate, chinomethionat, cryolite, dicofol, flufenerim, flometoquin, fluensulfone, flupyradifurone, piperonyl butoxide, pyridalyl, pyrifluquinazon, sulfoxaflor, or the compound
M.X.1: 4-[5-(3,5-Dichloro-phenyl)-5-trifluoromethyl-4,5-dihydro-isoxazol-3-yl]-2-methyl-N-[(2,2,2-trifluoro-ethylcarbamoyl)-methyl]-benzamide, or the compound
M.X.2: cyclopropaneacetic acid, 1,1′-[(3S,4R,4aR,6S,6aS,12R,12aS,12bS)-4-[[(2-cyclopropylacetyl)oxy]methyl]-1,3,4,4a,5,6,6a,12,12a,12b-decahydro-12-hydroxy-4,6a,12b-trimethyl-11-oxo-9-(3-pyridinyl)-2H,11H-naphtho[2,1-b]pyrano[3,4-e]pyran-3,6-diyl]ester, or the compound
M.X.3: 11-(4-chloro-2,6-dimethylphenyl)-12-hydroxy-1,4-dioxa-9-azadispiro[4.2.4.2]-tetradec-11-en-10-one, or the compound
M.X.4: 3-(4′-fluoro-2,4-dimethylbiphenyl-3-yl)-4-hydroxy-8-oxa-1-azaspiro[4.5]dec-3-en-2-one, or the compound
M.X.5: 1-[2-fluoro-4-methyl-5-[(2,2,2-trifluoroethyl)sulfinyl]phenyl]-3-(trifluoromethyl)-1H-1,2,4-triazole-5-amine, or actives on basis of bacillus firmus (Votivo, I-1582).

The commercially available compounds of the group M listed above may be found in The Pesticide Manual, 15th Edition, C. D. S. Tomlin, British Crop Protection Council (2011) among other publications.

The phthalamides M.28.1 and M.28.2 are both known from WO 2007/101540. The anthranilamide M.28.3 has been described in WO2005/077943. The hydrazide compound M.28.4 has been described in WO 2007/043677. The quinoline derivative flometoquin is shown in WO2006/013896. The aminofuranone compounds flupyradifurone is known from WO 2007/115644. The sulfoximine compound sulfoxaflor is known from WO2007/149134. The isoxazoline compound M.X.1 has been described in WO2005/085216. The pyripyropene derivative M.X.2 has been described in WO 2006/129714. The spiroketal-substituted cyclic ketoenol derivative M.X.3 is known from WO2006/089633 and the biphenyl-substituted spirocyclic ketoenol derivative M.X.4 from WO2008/067911. Finally triazoylphenylsulfide like M.X.5 have been described in WO2006/043635 and biological control agents on basis of bacillus firmus in WO2009/124707.

The following list of active fungicidal substances, in conjunction with which the compounds according to the invention can be used, is intended to illustrate the possible combinations but does not limit them:

F.I) Respiration Inhibitors

F.I-1) Inhibitors of complex III at Qo site (e.g. strobilurins)

strobilurins: azoxystrobin, dimoxystrobin, enestroburin, fluoxastrobin, kresoximmethyl, metominostrobin, orysastrobin, picoxystrobin, pyraclostrobin, pyrametostrobin, pyraoxystrobin, pyribencarb, trifloxystrobin, methyl(2-chloro-5[1-(3-methylbenzyloxyimino)ethyl]benzyl)carbamate and 2 (2-(3-(2,6-dichlorophenyl)-1-methyl-allylideneaminooxymethyl)-phenyl)-2-methoxyimino-N methyl-acetamide; oxazolidinediones and imidazolinones: famoxadone, fenamidone;

F.I-2) Inhibitors of complex II (e.g. carboxamides):

carboxanilides: benodanil, bixafen, boscalid, carboxin, fenfuram, fenhexamid, fluopyram, flutolanil, furametpyr, isopyrazam, isotianil, mepronil, oxycarboxin, penflufen, penthiopyrad, sedaxane, tecloftalam, thifluzamide, tiadinil, 2-amino-4 methyl-thiazole-5-carboxanilide, N-(3′,4′,5′ trifluorobiphenyl-2 yl)-3-difluoromethyl-1-methyl-1H-pyrazole-4 carboxamide, N-(4′-trifluoromethylthiobiphenyl-2-yl)-3 difluoromethyl-1-methyl-1H pyrazole-4-carboxamide and N-(2-(1,3,3-trimethyl-butyl)-phenyl)-1,3-dimethyl-5 fluoro-1H-pyrazole-4 carboxamide;

F.I-3) Inhibitors of complex III at Qi site: cyazofamid, amisulbrom;

F.I-4) Other respiration inhibitors (complex I, uncouplers)

diflumetorim; tecnazen; ferimzone; ametoctradin; silthiofam;

nitrophenyl derivates: binapacryl, dinobuton, dinocap, fluazinam, nitrthal-isopropyl, organometal compounds: fentin salts, such as fentin-acetate, fentin chloride or fentin hydroxide;

F.II) Sterol biosynthesis inhibitors (SBI fungicides)

F.II-1) C14 demethylase inhibitors (DMI fungicides, e.g. triazoles, imidazoles)

triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, paclobutrazole, penconazole, propiconazole, prothioconazole, simeconazole, tebuconazole, tetraconazole, triadimefon, triadimenol, triticonazole, uniconazole; imidazoles: imazalil, pefurazoate, oxpoconazole, prochloraz, triflumizole; pyrimidines, pyridines and piperazines: fenarimol, nuarimol, pyrifenox, triforine;

F.II-2) Delta14-reductase inhitors (Amines, e.g. morpholines, piperidines)

morpholines: aldimorph, dodemorph, dodemorph-acetate, fenpropimorph, tridemorph;

piperidines: fenpropidin, piperalin;

spiroketalamines: spiroxamine;

F.II-3) Inhibitors of 3-keto reductase: hydroxyanilides: fenhexamid;

F.III) Nucleic acid synthesis inhibitors

F.III-1) RNA, DNA synthesis

phenylamides or acyl amino acid fungicides: benalaxyl, benalaxyl-M, kiralaxyl, metalaxyl, metalaxyl-M (mefenoxam), ofurace, oxadixyl;

isoxazoles and iosothiazolones: hymexazole, octhilinone;

F.III-2) DNA topisomerase inhibitors: oxolinic acid;

F.III-3) Nucleotide metabolism (e.g. adenosin-deaminase)

hydroxy (2-amino)-pyrimidines: bupirimate;

F.IV) Inhibitors of cell division and or cytoskeleton

F.IV-1) Tubulin inhibitors: benzimidazoles and thiophanates: benomyl, carbendazim, fuberidazole, thiabendazole, thiophanatemethyl;

triazolopyrimidines: 5-chloro-7 (4-methylpiperidin-1-yl)-6-(2,4,6-trifluorophenyl)-[1,2,4]triazolo[1,5 a]pyrimidine

F.IV-2) Other cell division inhibitors

benzamides and phenyl acetamides: diethofencarb, ethaboxam, pencycuron, fluopicolide, zoxamide;

F.IV-3) Actin inhibitors: benzophenones: metrafenone;

F.V) Inhibitors of amino acid and protein synthesis

F.V-1) Mmethionine synthesis inhibitors (anilino-pyrimidines) anilino-pyrimidines: cyprodinil, mepanipyrim, nitrapyrin, pyrimethanil;

F.V-2) Protein synthesis inhibitors (anilino-pyrimidines)

antibiotics: blasticidin-S, kasugamycin, kasugamycin hydrochloride-hydrate, mildiomycin, streptomycin, oxytetracyclin, polyoxine, validamycin A;

F.VI) Signal transduction inhibitors

F.VI-1) MAP/Histidine kinase inhibitors (e.g. anilino-pyrimidines)

dicarboximides: fluoroimid, iprodione, procymidone, vinclozolin;

phenylpyrroles: fenpiclonil, fludioxonil;

F.VI-2) G protein inhibitors: quinolines: quinoxyfen;

F.VII) Lipid and membrane synthesis inhibitors

F.VII-1) Phospholipid biosynthesis inhibitors

organophosphorus compounds: edifenphos, iprobenfos, pyrazophos;

dithiolanes: isoprothiolane;

F.VII-2) Lipid peroxidation

aromatic hydrocarbons: dicloran, quintozene, tecnazene, tolclofos-methyl, biphenyl, chloroneb, etridiazole;

F.VII-3) Carboxyl acid amides (CAA fungicides)

cinnamic or mandelic acid amides: dimethomorph, flumorph, mandiproamid, pyrimorph; valinamide carbamates: benthiavalicarb, iprovalicarb, pyribencarb, valifenalate and N-(1-(1-(4-cyano-phenyl)ethanesulfonyl)-but-2-yl) carbamic acid-(4-fluorophenyl) ester;

F.VII-4) Compounds affecting cell membrane permeability and fatty acides carbamates: propamocarb, propamocarbhydrochlorid

F.VIII) Inhibitors with Multi Site Action

F.VIII-1) Inorganic active substances: Bordeaux mixture, copper acetate, copper hydroxide, copper oxychloride, basic copper sulfate, sulfur;

F.VIII-2) Thio- and dithiocarbamates: ferbam, mancozeb, maneb, metam, methasulphocarb, metiram, propineb, thiram, zineb, ziram;

F.VIII-3) Organochlorine compounds (e.g. phthalimides, sulfamides, chloronitriles): anilazine, chlorothalonil, captafol, captan, folpet, dichlofluanid, dichlorophen, flusulfamide, hexachlorobenzene, pentachlorphenole and its salts, phthalide, tolylfluanid, N-(4-chloro-2-nitro-phenyl)-N-ethyl-4-methyl-benzenesulfonamide;

F.VIII-4) Guanidines: guanidine, dodine, dodine free base, guazatine, guazatine-acetate, iminoctadine, iminoctadine-triacetate, iminoctadine-tris(albesilate);

F.VIII-5) Ahtraquinones: dithianon;

F.IX) Cell wall synthesis inhibitors

F.IX-1) Inhibitors of glucan synthesis: validamycin, polyoxin B;

F.IX-2) Melanin synthesis inhibitors: pyroquilon, tricyclazole, carpropamide, dicyclomet, fenoxanil;

F.X) Plant defence inducers

F.X-1) Salicylic acid pathway: acibenzolar-S-methyl;

F.X-2) Others: probenazole, isotianil, tiadinil, prohexadione-calcium; phosphonates: fosetyl, fosetyl-aluminum, phosphorous acid and its salts;

F.XI) Unknown mode of action:

bronopol, chinomethionat, cyflufenamid, cymoxanil, dazomet, debacarb, diclomezine, difenzoquat, difenzoquat-methylsulfate, diphenylamin, flumetover, flusulfamide, flutianil, methasulfocarb, oxin-copper, proquinazid, tebufloquin, tecloftalam, triazoxide, 2-butoxy-6-iodo-3-propylchromen-4-one, N-(cyclopropylmethoxyimino-(6-difluoromethoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N methyl formamidine, N′ (4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethylsilanyl-propoxy)-phenyl)-N-ethyl-N-methyl formamidine, N′-(5-difluoromethyl-2 methyl-4-(3-trimethylsilanyl-propoxy)-phenyl)-N-ethyl-N-methyl formamidine, 2-{1-[2-(5-methyl-3-trifluoromethyl-pyrazole-1-yl)-acetyl]-piperidin-4-yl}-thiazole-4-carboxylic acid methyl-(1,2,3,4-tetrahydro-naphthalen-1-yl)-amide, 2-{1-[2-(5-methyl-3-trifluoromethyl-pyrazole-1-yl)-acetyl]-piperidin-4-yl}-thiazole-4-carboxylic acid methyl-(R)-1,2,3,4-tetrahydro-naphthalen-1-yl-amide, methoxyacetic acid 6-tert-butyl-8-fluoro-2,3-dimethyl-quinolin-4-yl ester and N-Methyl-2-{1-[(5-methyl-3-trifluoromethyl-1H-pyrazol-1-yl)-acetyl]-piperidin-4-yl}-N-[(1R)-1,2,3,4-tetrahydronaphthalen-1-yl]-4-thiazolecarboxamide, 3-[5-(4-chloro-phenyl)-2,3-dimethyl-isoxazolidin-3 yl]-pyridine, 3-[5-(4-methyl-phenyl)-2,3-dimethyl-isoxazolidin-3-yl]-pyridine, 5-amino-2-isopropyl-3-oxo-4-ortho-tolyl-2,3-dihydro-pyrazole-1 carbothioic acid S-allyl ester, N-(6-methoxy-pyridin-3-yl)cyclopropanecarboxylic acid amide, 5-chloro-1 (4,6-dimethoxy-pyrimidin-2-yl)-2-methyl-1H-benzoimidazole, 2-(4-chloro-phenyl)-N-[4-(3,4-dimethoxy-phenyl)-isoxazol-5-yl]-2-prop-2-ynyloxy-acetamide;

F.XI) Growth regulators:

abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N 6 benzyladenine, paclobutrazol, prohexadione (prohexadione-calcium), prohydrojasmon, thidiazuron, triapenthenol, tributyl phosphorotrithioate, 2,3,5 tri iodobenzoic acid, trinexapacethyl and uniconazole;

F.XII) Biological control agents

antifungal biocontrol agents: Bacillus substilis strain with NRRL No. B-21661 (e.g. RHAPSODY®, SERENADE® MAX and SERENADE® ASO from AgraQuest, Inc., USA.), Bacillus pumilus strain with NRRL No. B-30087 (e.g. SONATA® and BALLAD® Plus from AgraQuest, Inc., USA), Ulocladium oudemansii (e.g. the product BOTRY-ZEN from BotriZen Ltd., New Zealand), Chitosan (e.g. ARMOUR-ZEN from BotriZen Ltd., New Zealand).

### Applications

The animal pest, i.e. the insects, arachnids and nematodes, the plant, soil or water in which the plant is growing can be contacted with the present compounds of formula I or composition(s) containing them by any application method known in the art. As such, “contacting” includes both direct contact (applying the compounds/compositions directly on the animal pest or plant—typically to the foliage, stem or roots of the plant) and indirect contact (applying the compounds/compositions to the locus of the animal pest or plant).

The compounds of formula I or the pesticidal compositions comprising them may be used to protect growing plants and crops from attack or infestation by animal pests, especially insects, acaridae or arachnids by contacting the plant/crop with a pesticidally effective amount of compounds of formula I. The term “crop” refers both to growing and harvested crops.

The compounds of the present invention and the compositions comprising them are particularly important in the control of a multitude of insects on various cultivated plants, such as cereal, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

The compounds of the present invention are employed as such or in form of compositions by treating the insects or the plants, plant propagation materials, such as seeds, soil, surfaces, materials or rooms to be protected from insecticidal attack with a insecticidally effective amount of the active compounds. The application can be carried out both before and after the infection of the plants, plant propagation materials, such as seeds, soil, surfaces, materials or rooms by the insects.

The present invention also includes a method of combating animal pests which comprises contacting the animal pests, their habit, breeding ground, food supply, cultivated plants, seed, soil, area, material or environment in which the animal pests are growing or may grow, or the materials, plants, seeds, soils, surfaces or spaces to be protected from animal attack or infestation with a pesticidally effective amount of a mixture of at least one active compound I.

Moreover, animal pests may be controlled by contacting the target pest, its food supply, habitat, breeding ground or its locus with a pesticidally effective amount of compounds of formula I. As such, the application may be carried out before or after the infection of the locus, growing crops, or harvested crops by the pest.

The compounds of the invention can also be applied preventively to places at which occurrence of the pests is expected.

The compounds of formula I may be also used to protect growing plants from attack or infestation by pests by contacting the plant with a pesticidally effective amount of compounds of formula I. As such, “contacting” includes both direct contact (applying the compounds/compositions directly on the pest and/or plant—typically to the foliage, stem or roots of the plant) and indirect contact (applying the compounds/compositions to the locus of the pest and/or plant).

“Locus” means a habitat, breeding ground, plant, seed, soil, area, material or environment in which a pest or parasite is growing or may grow.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “cultivated plants” is to be understood as including plants which have been modified by breeding, mutagenesis or genetic engineering. Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-transtional modification of protein(s) (oligo- or polypeptides) poly for example by glycosylation or polymer additions such as prenylated, acetylated or farnesylated moieties or PEG moieties (e.g. as disclosed in Biotechnol Prog. 2001 July-August; 17(4):720-8., Protein Eng Des Sel. 2004 January; 17(1):57-66, Nat Protoc. 2007;2(5):1225-35., Curr Opin Chem Biol. 2006 October; 10(5):487-91. Epub 2006 Aug. 28., Biomaterials. 2001 March; 22(5):405-17, Bioconjug Chem. 2005 January-February; 16(1):113-21).

The term “cultivated plants” is to be understood also including plants that have been rendered tolerant to applications of specific classes of herbicides, such as hydroxyphenylpyruvate dioxygenase (HPPD) inhibitors; acetolactate synthase (ALS) inhibitors, such as sulfonyl ureas (see e. g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073) or imidazolinones (see e. g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073); enolpyruvylshikimate-3-phosphate synthase (EPSPS) inhibitors, such as glyphosate (see e. g. WO 92/00377); glutamine synthetase (GS) inhibitors, such as glufosinate (see e. g. EP-A-0242236, EP-A-242246) or oxynil herbicides (see e. g. U.S. Pat. No. 5,559,024) as a result of conventional methods of breeding or genetic engineering. Several cultivated plants have been rendered tolerant to herbicides by conventional methods of breeding (mutagenesis), for example Clearfield® summer rape (Canola) being tolerant to imidazolinones, e. g. imazamox. Genetic engineering methods have been used to render cultivated plants, such as soybean, cotton, corn, beets and rape, tolerant to herbicides, such as glyphosate and glufosinate, some of which are commercially available under the trade names RoundupReady® (glyphosate) and LibertyLink® (glufosinate).

The term “cultivated plants” is to be understood also including plants that are by the use of recombinant DNA techniques capable to synthesize one or more insecticidal proteins, especially those known from the bacterial genus Bacillus, particularly from Bacillus thuringiensis, such as ä-endotoxins, e. g. CryIA(b), CryIA(c), CryIF, CryIF(a2), CryIIA(b), CryIIIA, CryIIIB(b1) or Cry9c; vegetative insecticidal proteins (VIP), e. g. VIP1, VIP2, VIP3 or VIP3A; insecticidal proteins of bacteria colonizing nematodes, for example Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, for example WO 02/015701). Further examples of such toxins or genetically-modified plants capable of synthesizing such toxins are dis-closed, for example, in EP-A 374 753, WO 93/007278, WO 95/34656, EP-A 427 529, EP-A 451 878, WO 03/018810 and WO 03/052073. The methods for producing such genetically modified plants are generally known to the person skilled in the art and are described, for example, in the publications mentioned above. These insecticidal proteins contained in the genetically modified plants impart to the plants producing these proteins protection from harmful pests from certain taxonomic groups of arthropods, particularly to beetles (Coleoptera), flies (Diptera), and butterflies and moths (Lepidoptera) and to plant parasitic nematodes (Nematoda).

The term “cultivated plants” is to be understood also including plants that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, for example EP-A 0 392 225), plant disease resistance genes (for example potato cultivars, which express resistance genes acting against Phytophthora infestans derived from the mexican wild potato Solanum bulbocastanum) or T4-lyso-zym (e. g. potato cultivars capable of synthesizing these proteins with increased resistance against bacteria such as Erwinia amylvora). The methods for producing such genetically modified plants are generally known to the person skilled in the art and are described, for example, in the publications mentioned above.

The term “cultivated plants” is to be understood also including plants that are by the use of recombinant DNA techniques capable to synthesize one or more proteins to increase the productivity (e. g. bio mass production, grain yield, starch content, oil content or protein content), tolerance to drought, salinity or other growth-limiting environmental factors or tolerance to pests and fungal, bacterial or viral pathogens of those plants.

The term “cultivated plants” is to be understood also including plants that contain by the use of recombinant DNA techniques a modified amount of substances of content or new substances of content, specifically to improve human or animal nutrition, for ex-ample oil crops that produce health-promoting long-chain omega-3 fatty acids or unsaturated omega-9 fatty acids (e. g. Nexera® rape).

The term “cultivated plants” is to be understood also including plants that contain by the use of recombinant DNA techniques a modified amount of substances of content or new substances of content, specifically to improve raw material production, for example potatoes that produce increased amounts of amylopectin (e. g. Amflora® potato).

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

For use in treating crop plants, the rate of application of the active ingredients of this invention may be in the range of 0.1 g to 4000 g per hectare, desirably from 25 g to 600 g per hectare, more desirably from 50 g to 500 g per hectare.

The compounds of formula I are effective through both contact (via soil, glass, wall, bed net, carpet, plant parts or animal parts), and ingestion (bait, or plant part).

The compounds of the invention may also be applied against non-crop insect pests, such as ants, termites, wasps, flies, mosquitos, crickets, or cockroaches. For use against said non-crop pests, compounds of formula I are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickyness, moisture retention or aging characteristics.

The bait employed in the composition is a product, which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

For use in bait compositions, the typical content of active ingredient is from 0.001 weight % to 15 weight %, desirably from 0.001 weight % to 5% weight % of active compound.

Formulations of compounds of formula I as aerosols (e.g in spray cans), oil sprays or pump sprays are highly suitable for the non-professional user for controlling pests such as flies, fleas, ticks, mosquitos or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulfoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

For use in spray compositions, the content of active ingredient is from 0.001 to 80 weights %, preferably from 0.01 to 50 weight % and most preferably from 0.01 to 15 weight %.

The compounds of formula I and its respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of formula I and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder. Suitable repellents for example are N,N-Diethyl-meta-toluamide (DEET), N,N-diethylphenylacetamide (DEPA), 1-(3-cyclohexan-1-yl-carbonyl)-2-methylpiperine, (2-hydroxymethylcyclohexyl) acetic acid lactone, 2-ethyl-1,3-hexandiol, indalone, Methylneodecanamide (MNDA), a pyrethroid not used for insect control such as {(+/−)-3-allyl-2-methyl-4-oxocyclopent-2-(+)-enyl-(+)-trans-chrysantemate (Esbiothrin), a repellent derived from or identical with plant extracts like limonene, eugenol, (+)-Eucamalol (1), (−)-1-epi-eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono- and di-ethylenically unsaturated hydrocarbons, such as styrene, and aliphatic diens, such as butadiene.

The impregnation of curtains and bednets is done in general by dipping the textile material into emulsions or dispersions of the insecticide or spraying them onto the nets.

The compounds of formula I and its compositions can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of formula I are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

### Seed Treatment

The compounds of formula I are also suitable for the treatment of seeds in order to protect the seed from insect pest, in particular from soil-living insect pests and the resulting plant's roots and shoots against soil pests and foliar insects.

The compounds of formula I are particularly useful for the protection of the seed from soil pests and the resulting plant's roots and shoots against soil pests and foliar insects. The protection of the resulting plant's roots and shoots is preferred. More preferred is the protection of resulting plant's shoots from piercing and sucking insects, wherein the protection from aphids is most preferred.

The present invention therefore comprises a method for the protection of seeds from insects, in particular from soil insects and of the seedling's roots and shoots from insects, in particular from soil and foliar insects, said method comprising contacting the seeds before sowing and/or after pregermination with a compound of the general formula I or a salt thereof. Particularly preferred is a method, wherein the plant's roots and shoots are protected, more preferably a method, wherein the plants shoots are protected form piercing and sucking insects, most preferably aa method, wherein the plants shoots are protected from aphids.

The term seed embraces seeds and plant propagules of all kinds including but not limited to true seeds, seed pieces, suckers, corms, bulbs, fruit, tubers, grains, cuttings, cut shoots and the like and means in a preferred embodiment true seeds.

The term seed treatment comprises all suitable seed treatment techniques known in the art, such as seed dressing, seed coating, seed dusting, seed soaking and seed pelleting.

The present invention also comprises seeds coated with or containing the active compound.

The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the active compound may also be used for the treatment seeds from plants, which tolerate the action of herbicides or fungicides or insecticides owing to breeding, including genetic engineering methods.

For example, the active compound can be employed in treatment of seeds from plants, which are resistant to herbicides from the group consisting of the sulfonylureas, imidazolinones, glufosinate-ammonium or glyphosate-isopropylammonium and analogous active substances (see for example, EP-A-0242236, EP-A-242246) (WO 92/00377) (EP-A-0257993, U.S. Pat. No. 5,013,659) or in transgenic crop plants, for example cotton, with the capability of producing Bacillus thuringiensis toxins (Bt toxins) which make the plants resistant to certain pests (EP-A-0142924, EP-A-0193259),

Furthermore, the active compound can be used also for the treatment of seeds from plants, which have modified characteristics in comparison with existing plants consist, which can be generated for example by traditional breeding methods and/or the generation of mutants, or by recombinant procedures). For example, a number of cases have been described of recombinant modifications of crop plants for the purpose of modifying the starch synthesized in the plants (e.g. WO 92/11376, WO 92/14827, WO 91/19806) or of transgenic crop plants having a modified fatty acid composition (WO 91/13972).

The seed treatment application of the active compound is carried out by spraying or by dusting the seeds before sowing of the plants and before emergence of the plants.

Compositions which are especially useful for seed treatment are e.g.:

A Soluble concentrates (SL, LS)

D Emulsions (EW, EO, ES)

E Suspensions (SC, OD, FS)

F Water-dispersible granules and water-soluble granules (WG, SG)

G Water-dispersible powders and water-soluble powders (WP, SP, WS)

H Gel-Formulations (GF)

I Dustable powders (DP, DS)

Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter

In a preferred embodiment a FS formulation is used for seed treatment. Typcially, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Especially preferred FS formulations of compounds of formula I for seed treatment usually comprise from 0.1 to 80% by weight (1 to 800 g/l) of the active ingredient, from 0.1 to 20% by weight (1 to 200 g/l) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

Seed Treatment formulations may additionally also comprise binders and optionally colorants.

Binders can be added to improve the adhesion of the active materials on the seeds after treatment. Suitable binders are homo- and copolymers from alkylene oxides like ethylene oxide or propylene oxide, polyvinylacetate, polyvinylalcohols, polyvinylpyrrolidones, and copolymers thereof, ethylene-vinyl acetate copolymers, acrylic homo- and copolymers, polyethyleneamines, polyethyleneamides and polyethyleneimines, polysaccharides like celluloses, tylose and starch, polyolefin homo- and copolymers like olefin/maleic anhydride copolymers, polyurethanes, polyesters, polystyrene homo and copolymers

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of a gelling agent is carrageen (Satiagel®)

In the treatment of seed, the application rates of the compounds I are generally from 0.1 g to 10 kg per 100 kg of seed, preferably from 1 g to 5 kg per 100 kg of seed, more preferably from 1 g to 1000 g per 100 kg of seed and in particular from 1 g to 200 g per 100 kg of seed.

The invention therefore also relates to seed comprising a compound of the formula I, or an agriculturally useful salt of I, as defined herein. The amount of the compound I or the agriculturally useful salt thereof will in general vary from 0.1 g to 10 kg per 100 kg of seed, preferably from 1 g to 5 kg per 100 kg of seed, in particular from 1 g to 1000 g per 100 kg of seed. For specific crops such as lettuce the rate can be higher.

### Animal Health

The compounds of formula I or the enantiomers or veterinarily acceptable salts thereof are in particular also suitable for being used for combating parasites in and on animals.

An object of the present invention is therfore also to provide new methods to control parasites in and on animals. Another object of the invention is to provide safer pesticides for animals. Another object of the invention is further to provide pesticides for animals that may be used in lower doses than existing pesticides. And another object of the invention is to provide pesticides for animals, which provide a long residual control of the parasites.

The invention also relates to compositions containing a parasiticidally effective amount of compounds of formula I or the enantiomers or veterinarily acceptable salts thereof and an acceptable carrier, for combating parasites in and on animals.

The present invention also provides a method for treating, controlling, preventing and protecting animals against infestation and infection by parasites, which comprises orally, topically or parenterally administering or applying to the animals a parasiticidally effective amount of a compound of formula I or the enantiomers or veterinarily acceptable salts thereof or a composition comprising it.

The invention also provides a process for the preparation of a composition for treating, controlling, preventing or protecting animals against infestation or infection by parasites which comprises a parasiticidally effective amount of a compound of formula I or the enantiomers or veterinarily acceptable salts thereof or a composition comprising it.

Activity of compounds against agricultural pests does not suggest their suitability for control of endo- and ectoparasites in and on animals which requires, for example, low, non-emetic dosages in the case of oral application, metabolic compatibility with the animal, low toxicity, and a safe handling.

Surprisingly it has now been found that compounds of formula I are suitable for combating endo- and ectoparasites in and on animals.

Compounds of formula I or the enantiomers or veterinarily acceptable salts thereof and compositions comprising them are preferably used for controlling and preventing infestations and infections animals including warm-blooded animals (including humans) and fish. They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels.

Compounds of formula I or the enantiomers or veterinarily acceptable salts thereof and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of formula I or the enantiomers or veterinarily acceptable salts thereof and compositions comprising them are suitable for systemic and/or non-systemic control of ecto- and/or endoparasites. They are active against all or some stages of development.

The compounds of formula I are especially useful for combating ectoparasites.

The compounds of formula I are especially useful for combating parasites of the following orders and species, respectively:

fleas (Siphonaptera), e.g. Ctenocephalides felis, Ctenocephalides canis, Xenopsylla cheopis, Pulex irritans, Tunga penetrans, and Nosopsyllus fasciatus, cockroaches (Blattaria—Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,

flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrirnaculatus, Calliphora vicina, Chrysomya bezziana, Chrysomya hominivorax, Chrysomya macellana, Chrysops Chrysops silacea, Chrysops atlanticus, Cochliompa hominivorax, Cordylobia anthropophaga, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophllus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosirnulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis,

lice (Phthiraptera), e.g. Pediculus humanus capitis, Pediculus humanus corporis, Pthirus Haematopinus eurysternus, Haematopinus suis, Linognathus vituli, Bovicola bovis, Menopon gallinae, Menacanthus stramineus and Solenopotes capillatus.

ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor vanabllis, Amblyomma americanum, Ambryomma maculatum, Ornithodorus hermsi, Ornithodorus turicata and parasitic mites (Mesostigmata), e.g. Ornithonyssus Bacoti and Dermanyssus gallinae,

Actinedida (Prostigmata) and Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp,

Bugs (Heteropterida): Cimex lectularius, Cimex hemipterus, Reduvius senilis, Triatoma spp., Rhodnius ssp., Panstrongylus ssp. and Arilus critatus,

Anoplurida, e.g. Haematopinus spp., Linognathus spp., Pediculus spp., Phtirus spp., and Solenopotes spp,

Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp,

Roundworms Nematoda:

Wipeworms and Trichinosis (Trichosyringida), e.g. Trichinellidae (Trichinella spp.), (Trichuridae) Trichuris spp., Capillaria spp,

Rhabditida, e.g. Rhabditis spp, Strongyloides spp., Hekephalobus spp,

Strongylida, e.g. Strongylus spp., Ancylostoma spp., Necator americanus, Bunostomum spp. (Hookworm), Trichostrongylus spp., Haemonchus contortus., Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinana spp., Globocephalus spp., Necator spp., Metastrongylus spp., Muellerius capillaris, Protostrongylus spp., Angiostrongylus spp., Parelaphostrongylus spp. Aleurostrongylus abstrusus, and Dioctophyma resale,

Intestinal roundworms (Ascaridida), e.g. Ascaris lumbricoides, Ascaris suum, Ascandia galli, Parascaris equorum, Enterobius vermicularis (Threadworm), Toxocara canis, Toxascaris leonine, Skrjabinema spp., and Oxyuris equi,

Camallanida, e.g. Dracunculus medinensis (guinea worm)

Spirurida, e.g. Thelazia spp. Wuchereria spp., Brugia spp., Onchocerca spp., Dirofilari spp.a, Dipetalonema spp., Setaria spp., Elaeophora spp., Spirocerca lupi, and Habronema spp.,

Thorny headed worms (Acanthocephala), e.g. Acanthocephalus spp., Macracanthorhynchus hirudinaceus and Oncicola spp,

Planarians (Plathelminthes):

Flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alana alata, Paragonimus spp., and Nanocyetes spp,

Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

The compounds of formula I and compositions containing them are particularly useful for the control of pests from the orders Diptera, Siphonaptera and Ixodida.

Moreover, the use of the compounds of formula I and compositions containing them for combating mosquitoes is especially preferred.

The use of the compounds of formula I and compositions containing them for combating flies is a further preferred embodiment of the present invention.

Furthermore, the use of the compounds of formula I and compositions containing them for combating fleas is especially preferred.

The use of the compounds of formula I and compositions containing them for combating ticks is a further preferred embodiment of the present invention.

The compounds of formula I also are especially useful for combating endoparasites (roundworms nematoda, thorny headed worms and planarians).

Administration can be carried out both prophylactically and therapeutically.

Administration of the active compounds is carried out directly or in the form of suitable preparations, orally, topically/dermally or parenterally.

For oral administration to warm-blooded animals, the formula I compounds may be formulated as animal feeds, animal feed premixes, animal feed concentrates, pills, solutions, pastes, suspensions, drenches, gels, tablets, boluses and capsules. In addition, the formula I compounds may be administered to the animals in their drinking water. For oral administration, the dosage form chosen should provide the animal with 0.01 mg/kg to 100 mg/kg of animal body weight per day of the formula I compound, preferably with 0.5 mg/kg to 100 mg/kg of animal body weight per day.

Alternatively, the formula I compounds may be administered to animals parenterally, for example, by intraruminal, intramuscular, intravenous or subcutaneous injection. The formula I compounds may be dispersed or dissolved in a physiologically acceptable carrier for subcutaneous injection. Alternatively, the formula I compounds may be formulated into an implant for subcutaneous administration. In addition the formula I compound may be transdermally administered to animals. For parenteral administration, the dosage form chosen should provide the animal with 0.01 mg/kg to 100 mg/kg of animal body weight per day of the formula I compound.

The formula I compounds may also be applied topically to the animals in the form of dips, dusts, powders, collars, medallions, sprays, shampoos, spot-on and pour-on formutations and in ointments or oil-in-water or water-in-oil emulsions. For topical application, dips and sprays usually contain 0.5 ppm to 5,000 ppm and preferably 1 ppm to 3,000 ppm of the formula I compound. In addition, the formula I compounds may be formulated as ear tags for animals, particularly quadrupeds such as cattle and sheep.

Suitable preparations are:
Solutions such as oral solutions, concentrates for oral administration after dilution, solutions for use on the skin or in body cavities, pouring-on formulations, gels;
Emulsions and suspensions for oral or dermal administration; semi-solid preparations;
Formulations in which the active compound is processed in an ointment base or in an oil-in-water or water-in-oil emulsion base;
Solid preparations such as powders, premixes or concentrates, granules, pellets, tablets, boluses, capsules; aerosols and inhalants, and active compound-containing shaped articles.

Compositions suitable for injection are prepared by dissolving the active ingredient in a suitable solvent and optionally adding further ingredients such as acids, bases, buffer salts, preservatives, and solubilizers. The solutions are filtered and filled sterile.

Suitable solvents are physiologically tolerable solvents such as water, alkanols such as ethanol, butanol, benzyl alcohol, glycerol, propylene glycol, polyethylene glycols, N-methyl-pyrrolidone, 2-pyrrolidone, and mixtures thereof.

The active compounds can optionally be dissolved in physiologically tolerable vegetable or synthetic oils which are suitable for injection.

Suitable solubilizers are solvents which promote the dissolution of the active compound in the main solvent or prevent its precipitation. Examples are polyvinylpyrrolidone, polyvinyl alcohol, polyoxyethylated castor oil, and polyoxyethylated sorbitan ester.

Suitable preservatives are benzyl alcohol, trichlorobutanol, p-hydroxybenzoic acid esters, and n-butanol.

Oral solutions are administered directly. Concentrates are administered orally after prior dilution to the use concentration. Oral solutions and concentrates are prepared according to the state of the art and as described above for injection solutions, sterile procedures not being necessary.

Solutions for use on the skin are trickled on, spread on, rubbed in, sprinkled on or sprayed on.

Solutions for use on the skin are prepared according to the state of the art and according to what is described above for injection solutions, sterile procedures not being necessary.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of application, and the like.

The compositions which can be used in the invention can comprise generally from about 0.001 to 95% of the compound of formula I.

Generally it is favorable to apply the compounds of formula I in total amounts of 0.5 mg/kg to 100 mg/kg per day, preferably 1 mg/kg to 50 mg/kg per day.

Ready-to-use preparations contain the compounds acting against parasites, preferably ectoparasites, in concentrations of 10 ppm to 80 per cent by weight, preferably from 0.1 to 65 per cent by weight, more preferably from 1 to 50 per cent by weight, most preferably from 5 to 40 per cent by weight.

Preparations which are diluted before use contain the compounds acting against ectoparasites in concentrations of 0.5 to 90 per cent by weight, preferably of 1 to 50 per cent by weight.

Furthermore, the preparations comprise the compounds of formula I against endoparasites in concentrations of 10 ppm to 2 per cent by weight, preferably of 0.05 to 0.9 per cent by weight, very particularly preferably of 0.005 to 0.25 per cent by weight.

In a preferred embodiment of the present invention, the compositions comprising the compounds of formula I them are applied dermally/topically.

In a further preferred embodiment, the topical application is conducted in the form of compound-containing shaped articles such as collars, medallions, ear tags, bands for fixing at body parts, and adhesive strips and foils.

Generally it is favorable to apply solid formulations which release compounds of formula I in total amounts of 10 mg/kg to 300 mg/kg, preferably 20 mg/kg to 200 mg/kg, most preferably 25 mg/kg to 160 mg/kg body weight of the treated animal in the course of three weeks.

For the preparation of the shaped articles, thermoplastic and flexible plastics as well as elastomers and thermoplastic elastomers are used. Suitable plastics and elastomers are polyvinyl resins, polyurethane, polyacrylate, epoxy resins, cellulose, cellulose derivatives, polyamides and polyester which are sufficiently compatible with the compounds of formula I. A detailed list of plastics and elastomers as well as preparation procedures for the shaped articles is given e.g. in WO 03/086075.

### EXAMPLES

The present invention is now illustrated in further details by the following examples, without imposing any limitation thereto.

### S. Synthesis Examples

### S.1. 3-[5-(1H-Pyrazol-3-yl)-4-trifluoromethyl-thiazol-2-yl]-pyridine

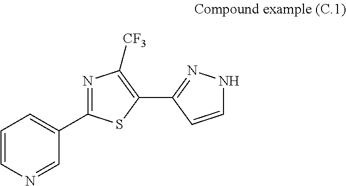 [SMILES: FC(F)(F)c1nc(-c2cccnc2)sc1-c1cc[nH]n1]

### Step 1.1: 2-Pyridin-3-yl-4-trifluoromethyl-thiazole-5-carboxylic acid methoxy-methyl-amide

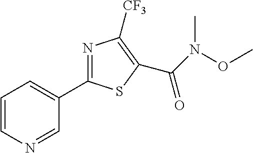 [SMILES: CON(C)C(=O)c1sc(-c2cccnc2)nc1C(F)(F)F]

To pyridin-3-yl-4-trifluoromethyl-thiazole-5-carboxylic acid (1.10 g, 4.0 mmol) was added thionyl chloride (15 mL) and the resulting solution was stirred at 80° C. for 3 h before concentrating in vacuo. The resulting acid chloride (4.0 mmol) was then dissolved in CH₂Cl₂ (10 mL) and added dropwise to a solution of dimethylhydroxylamine hydrochloride (663 mg, 6.8 mmol) and EtNPr₂ (3.77 mL, 22 mmol) in CH₂Cl₂ (20 mL) at 0° C. The reaction was stirred at ambient temperature for 16 h and then diluted with CH₂Cl₂ (20 mL) and washed with water (2×20 mL), dried (MgSO₄) and concentrated to afford the title compound (1.17 g, 92%).

HPLC-MS: Rₜ (min) and [M + H]

| | |
|---|---|
| R_{T} = 2.178 min (column 1) | (M + H) = 317 |

### Step 1.2: 1-(2-Pyridin-3-yl-4-trifluoromethyl-thiazol-5-yl)-ethanone

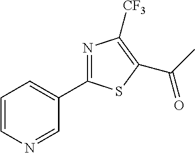 [SMILES: CC(=O)c1sc(-c2cccnc2)nc1C(F)(F)F]

To methylmagnesiumbromide (1.4 M in toluene/THF, 1 mL, 1.4 mmol) was added 2-pyridin-3-yl-4-trifluoromethyl-thiazole-5-carboxylic acid methoxy-methyl-amide (295 mg, 0.93 mmol) in CH₂Cl₂ (1 mL). The reaction was stirred at ambient temperature for 16 h and then quenched with saturated aqueous ammonium chloride solution (2 mL). The organic phase was then separated, dried (MgSO₄) and concentrated to afford the title compound (247 mg, 98%).

HPLC-MS: Rₜ (min) and [M + H]

| | |
|---|---|
| R_{T} = 2.365 min (column 1) | (M + H) = 273 |

### Step 1.3: (E)-3-Dimethylamino-1-(2-pyridin-3-yl-4-trifluoromethyl-thiazol-5-yl)-propenone

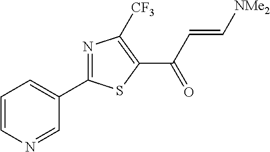 [SMILES: CN(C)/C=C/C(=O)c1sc(-c2cccnc2)nc1C(F)(F)F]

1-(2-Pyridin-3-yl-4-trifluoromethyl-thiazol-5-yl)-ethanone (727 mg, 2.76 mmol) and N,N-dimethylformamide dimethylacetal (428 mg, 3.59 mmol) were heated together for 16 h at 100° C. The reaction was then concentrated to afford the desired product (852 mg, 94%).

HPLC-MS: Rₜ (min) and [M + H]

| | |
|---|---|
| R_{T} = 0.807 min | (M + H) = 328 |

### Step 1.4: 3-[5-(1H-Pyrazol-3-yl)-4-trifluoromethyl-thiazol-2-yl]-pyridine

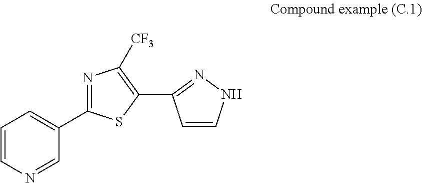 [SMILES: FC(F)(F)c1nc(-c2cccnc2)sc1-c1cc[nH]n1]

To (E)-3-dimethylamino-1-(2-pyridin-3-yl-4-trifluoromethyl-thiazol-5-yl)-propenone (282 mg, 0.91 mmol) in EtOH (10 mL) was added hydrazine hydrate (100 mg, 2.0 mmol). The reaction was heated to reflux for 1 h, then allowed to cool and concentrated in vacuo to afford the title compound (257 mg, 95%).

### S.2. 2-[3-(2-Pyridin-3-yl-4-trifluoromethyl-thiazol-5-yl)-pyrazol-1-yl]-pyrimidine

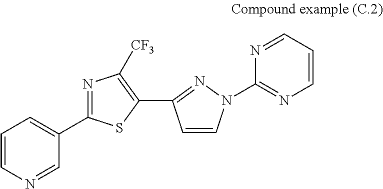 [SMILES: FC(F)(F)c1nc(-c2cccnc2)sc1-c1ccn(-c2ncccn2)n1]

To 3-[5-(1H-pyrazol-3-yl)-4-trifluoromethyl-thiazol-2-yl]pyridine (168 mg, 0.57 mmol) in DMF (5 mL) was added 2-chloropyrimidine (65 mg, 0.57 mmol) and sodium hydride (60% in mineral oil, 46 mg, 1.14 mmol). The reaction was heated to 80° C. for 48 h and then concentrated in vacuo. Column chromatography (cyclohexane/ethylacetate) afforded the desired product (91 mg, 43%).

### S.3 2-Phenyl-4-(2-pyridin-3-yl-4-trifluoromethyl-thiazol-5-yl)-pyrimidine

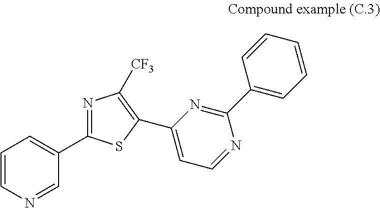 [SMILES: FC(F)(F)c1nc(-c2cccnc2)sc1-c1ccnc(-c2ccccc2)n1]

To (E)-3-dimethylamino-1-(2-pyridin-3-yl-4-trifluoromethyl-thiazol-5-yl)-Propenone (164 mg, 0.5 mmol) in EtOH (5 mL) was added benzamidine hydrochloride (86 mg, 0.55 mol) and sodium ethoxide (41 mg, 0.6 mmol). The reaction was heated at reflux for 48 h and then concentrated in vacuo. Column chromatography (cyclohexane/ethylacetate) afforded the desired product (83 mg, 44%).

### S.4 4-chloro-5-(2-pyridyl)-2-(3-pyridyl)thiazole

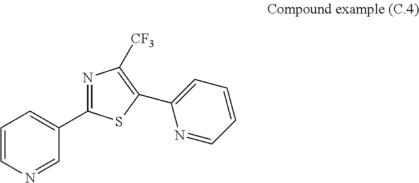 [SMILES: FC(F)(F)c1nc(-c2cccnc2)sc1-c1ccccn1]

### Step 4.1: 2-(3-pyridyl)thiazole

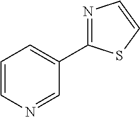 [SMILES: c1cncc(-c2nccs2)c1]

To a solution of thionicotinamide (15.3 g, 0.11 mol) in acetic acid (75 mL) at room temperature was added a solution of chloroacetaldehyde (34.5 g, 0.22 mol, 50% in water) in acetic anhydride (52 mL, 0.55 mol). The reaction mixture was heated at 70° C. for 16 h, allowed to cool to room temperature, and poured into a solution of ice/ammonium hydroxide. A saturated solution of aq. sodium chloride was then added and the mixture was stirred for 3 h. The mixture was then filtered and extracted with methyl t-butylether. The combined organic layers were dried (MgSO₄) then concentrated in vacuo. The concentrate was then tritiated with ethyl acetate, filtered and concentrated in vacuo. Column chromatography of the concentrate (cyclohexane/ethylacetate) afforded the desired product (2.48 g, 14%).

HPLC-MS: Rₜ (min) and [M + H]

| | |
|---|---|
| RT = 1.123 min (column 1) | (M + H) = 163 |

### Step 4.2: 5-(2-pyridyl)-2-(3-pyridyl)thiazole

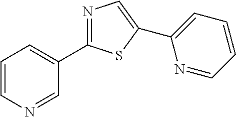 [SMILES: c1ccc(-c2cnc(-c3cccnc3)s2)nc1]

A suspension of palladium(II) chloride (35 mg, 0.2 mmol), tri(o-tolyl)phosphine (61 mg, 0.2 mmol), t-butylammonium chloride (56 mg, 0.2 mmol), 2-(3-pyridyl)thiazole (162 mg mg, 1 mmol), 2-bromopyridine (190 mg, 1.2 mmol) and potassium carbonate (263 mg, 1.9 mmol) in DMF (5 mL) under argon was heated at 120° C. for 16 h. The reaction mixture was then concentrated in vacuo, the concentrate was dissolved in water and extracted with ethyl acetate. The combined organic layers were dried (MgSO₄) and then concentrated in vacuo. Column chromatography (dichloromethane/methanol) afforded the desired product (70 mg, 29%).

HPLC-MS: Rₜ (min) and [M + H]

| | |
|---|---|
| RT = 1.887 min (column 2) | (M + H) = 240 |

### Step 4.3: 4-chloro-5-(2-pyridyl)-2-(3-pyridyl)thiazole

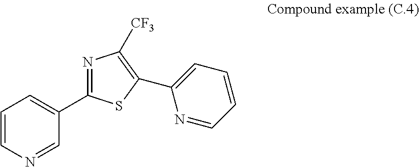 [SMILES: FC(F)(F)c1nc(-c2cccnc2)sc1-c1ccccn1]

A suspension of 5-(2-pyridyl)-2-(3-pyridyl)thiazole (69 mg, 0.29 mmol) and N-chlorosuccinimide (378 mg, 2.9 mmol) in DMF (1 mL) was heated at 120° C. for 2 d. The reaction mixture was then cooled to room temperature and concentrated in vacuo. The concentrate was then dissolved in water and extracted with ethyl acetate. The combined organic layers were dried (MgSO₄) and then concentrated in vacuo. Column chromatography (cyclohexane/ethylacetate) afforded the desired product (30 mg, 33%).

### C. Compound Examples

Some compound examples of the present invention are already shown in the synthesis examples above.

Additional examples are listed herein below:

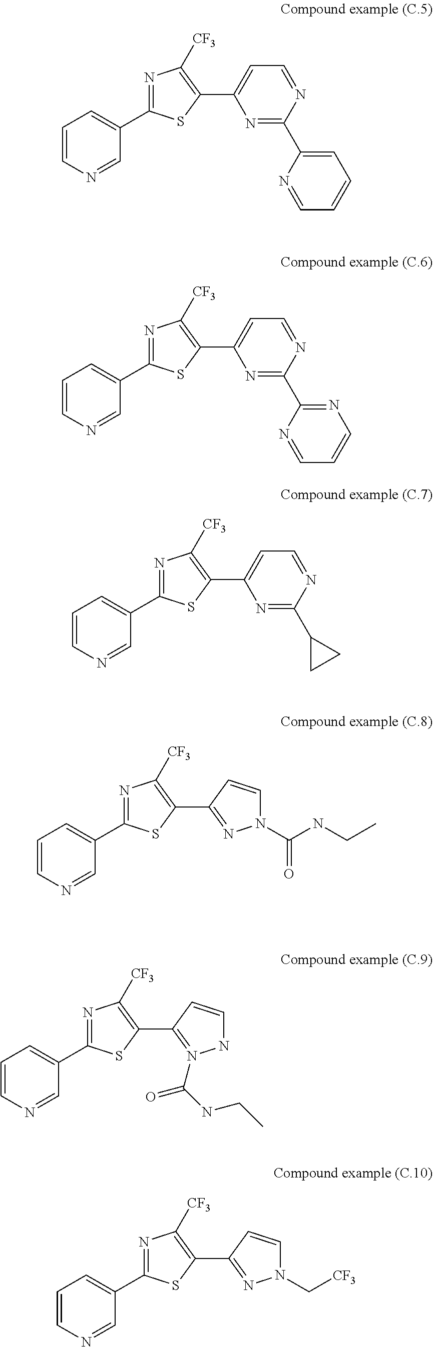

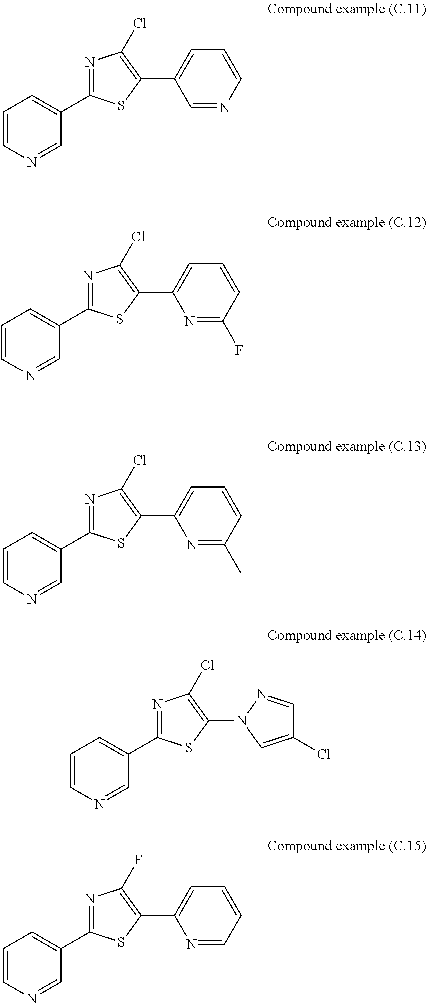 [SMILES: Clc1nc(-c2cccnc2)sc1-c1cccnc1 ; Fc1cccc(-c2sc(-c3cccnc3)nc2Cl)n1 ; Cc1cccc(-c2sc(-c3cccnc3)nc2Cl)n1 ; Clc1cnn(-c2sc(-c3cccnc3)nc2Cl)c1 ; Fc1nc(-c2cccnc2)sc1-c1ccccn1]

Compounds can in general be characterized e.g. by coupled High Performance Liquid Chromatography/mass spectrometry (HPLC/MS), by ¹H-NMR and/or by their melting points.

Analytical HPLC column 1: RP-18 column Chromolith Speed ROD from Merck KgaA, Germany). Elution: acetonitrile+0.1% trifluoroacetic acid (TFA)/water+0.1% trifluoroacetic acid (TFA) in a ratio of from 5:95 to 95:5 in 5 minutes at 40° C.

Rₜ or r.t.=HPLC retention time; m/z of the [M+H]+, [M+Na]+ or [M+K]+ peaks.

Analytical HPLC column 2: Phenomenex Kinetex 1.7 μm XB-C18 100A; 50×2.1 mm Elution: A: acetonitrile+0.1% trifluoroacetic acid (TFA)/water+0.1% trifluoroacetic acid (TFA) in a ratio of from 5:95 to 95:5 in 1.5 minutes at 50° C.

Rₜ or r.t.=HPLC retention time; m/z of the [M+H]+, [M+Na]+ or [M+K]+ peaks.

¹H-NMR, respectively ¹³C-NMR: The signals are characterized by chemical shift (ppm) vs. tetramethylsilane, respectively CDCl₃ for ¹³C-NMR, by their multiplicity and by their integral (relative number of hydrogen atoms given). The following abbreviations are used to characterize the multiplicity of the signals: m=multiplet, q=quartet, t=triplet, d=doublet and s=singlet.

### Characterization Data of Compound Examples:

TABLE P.1

| Compound Example. | HPLC-MS: Rₜ (min) and [M + H] | |
|---|---|---|
| C. 1 | Rₜ = 1.159 min (column 2) | (M + H) = 297 |
| C. 2 | Rₜ = 0.957 min (column 2) | (M + H) = 375 |
| C. 3 | Rₜ = 1.233 min (column 2) | (M + H) = 385 |
| C. 4 | Rₜ = 2.468 min (column 1) | (M + H) = 274 |
| C. 5 | Rₜ = 0.789 min (column 2) | (M + H) = 386 |
| C. 6 | Rₜ = 3.132 min (column 1) | (M + H) = 387 |
| C. 7 | Rₜ = 3.173 min (column 1) | (M + H) = 349 |
| C. 8 | Rₜ = 2.875 min (column 1) | (M + H) = 368 |
| C. 9 | Rₜ = 2.641 min (column 1) | (M + H) = 368 |
| C. 10 | Rₜ = 1.058 min (column 2) | (M + H) = 379 |
| C. 11 | Rₜ = 1.620 min (column 1) | (M + H) = 274 |
| C. 12 | Rₜ = 2.923 min (column 1) | (M + H) = 292 |
| C. 13 | Rₜ = 2.937 min (column 1) | (M + H) = 288 |
| C. 14 | Rₜ = 1.033 min (column 2) | (M + H) = 297 |
| C. 15 | Rₜ = 2.318 min (column 1) | (M + H) = 258 |

TABLE P.2

| Compound | |
|---|---|
| Example. | ¹H-NMR (400 MHz, CDCl₃) |
| C. 5 | NMR (500 MHz, CDCl3) 9.25 (s, 1H), 9.09 (d, J = 5 Hz, 1H), |
| | 8.88 (d, J = 3.5 Hz, 1H), 8.75 (d, J = 4 Hz, 1H), 8.57 (d, J = |
| | 8 Hz, 1H), 8.35 (d, J = 7.5 Hz, 1H), 7.92 (t, J = 6.5 Hz, 1H), |
| | 7.74 (d, J = 5 Hz, 1H), 7.47-7.44 (m, 2H). |
| C. 6 | NMR (500 MHz, CDCl₃) 9.25(s, 1H), 9.09 (d, J = 5 Hz, 1H), |
| | 8.88 (d, J = 3.5 Hz, 1H), 8.75 (d, J = 4 Hz, 1H), 8.57 (d, J = |
| | 8 Hz, 1H), 8.35 (d, J = 7.5 Hz, 1H), 7.92 (t, J = 6.5 Hz, 1H), |
| | 7.74 (d, J = 5 Hz, 1H), 7.47-7.44 (m, 2H). |
| C. 7 | NMR (500 MHz, CDCl₃) 9.25 (s, 1H), 9.17 (d, J = 6 Hz, |
| | 1H), 9.08 (d, J = 5 Hz, 2H), 8.76 (d, J = 5 Hz, 1H), 8.36 |
| | (d, J = 8.5 Hz, 1H), 7.87 (d, J = 5.5 Hz, 1H), 7.51-7.45 (m, 2H). |
| C. 8 | NMR (500 MHz, CDCl₃) 9.21 (s, 1H), 8.73 (d, J = 4.5 Hz, |
| | 1H), 8.69 (d, J = 4.5 Hz, 1H), 8.32 (d, J = 7.5 Hz, 1H), 7.48 |
| | (d, J = 5 Hz, 1H), 7.45 (dd, J = 8, 5 Hz, 1H), 2.32-2.29 (m, 1H), |
| | 1.22-1.20 (m, 2H), 1.16-1.14 (m, 2H). |
| C. 9 | NMR (500 MHz,CDCl₃) 9.23 (d, J = 1.5 Hz, 1H), 8.75 (dd, |
| | J = 5, 1.5 Hz, 1H), 8.48 (dt, J = 8.5, 1.5, 1H), 8.32 (d, J = 3 Hz, |
| | 1H), 7.59 (dd, J = 8, 5 Hz, 1H), 7.20-7.18 (m, 1H), 6.80 (dd, J = |
| | 2.5, 1.5 Hz, 1H), 3.55-3.52 (m, 2H), 1.34 (t, J = 7.5 Hz, 3H). |
| C. 10 | NMR (500 MHz, CDCl₃) 9.23 (d, J = 1.5 Hz, 1H), 8.80 (dd, |
| | J = 5, 2 Hz, 1H), 8.55 (dt, J = 8, 2, 1H), 7.69-7.67 (m, 2H), 6.63 |
| | (d, J = 1.5 Hz, 1H), 6.50 (s, 1H), 3.47-3.43 (m, 2H), 1.34 |
| | (t, J = 7.5 Hz, 3H). |

### B. Biological Examples

The biological activity of the compounds of formula I of the present invention can evaluated in biological tests as described in the following.

### General Conditions

If not otherwise specified, most test solutions are to be prepared as follows: The active compound is dissolved at the desired concentration in a mixture of 1:1 (vol:vol) distilled water: acteon. The test solutions are prepared at the day of use (and, if not otherwised specified, in general at concentrations wt/vol).

B.1 Green Peach Aphid (Myzus persicae)

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone:50% water (v/v). A nonionic surfactant (Kinetic®) was included in the solution at a volume of 0.01% (v/v).

Bell pepper plants at the first true-leaf stage were infested prior to treatment by placing heavily infested leaves from the main colony on top of the treatment plants. Aphids were allowed to transfer overnight to accomplish an infestation of 30-50 aphids per plant and the host leaves were removed. The infested plants were then sprayed by an automated electrostatic plant sprayer equipped with an atomizing spray nozzle. The plants were dried in the sprayer fume hood, removed, and then maintained in a growth room under fluorescent lighting in a 24-hr photoperiod at about 25° C. and about 20-40% relative humidity. Aphid mortality on the treated plants, relative to mortality on untreated control plants, was determined after 5 days.

In this test, the following compounds at 500 ppm showed a mortality of at least 75% in comparison with untreated controls: C.2, C.4, C.6, C.8, C.10, C.11, C.12, C.13, C.14, C.15.

B.2 Cotton Aphid (Aphis gossypii)

The active compounds were formulated in 50:50 (vol:vol) acetone: water and 100 ppm Kinetica™ surfactant.

Cotton plants at the cotyledon stage (one plant per pot) weare infested by placing a heavily infested leaf from the main colony on top of each cotyledon. The aphids were allowed to transfer to the host plant overnight, and the leaf used to transfer the aphids was removed. The cotyledons were dipped in the test solution and allowed to dry. After 5 days, mortality counts were made.

In this test, the following compounds at 500 ppm showed a mortality of at least 75% in comparison with untreated controls: C.2, C.6, C.8, C.10, C.11, C.12, C.13, C.14, C.15

B.3 Cowpea Aphid (Aphis craccivora)

The active compound was dissolved at the desired concentration in a mixture of 1:1 (vol:vol) distilled water:acetone. The test solution was prepared at the day of use.

Potted cowpea plants colonized with approximately 100-150 aphids of various stages were sprayed after the pest population had been recorded. Population reduction was assessed after 24, 72, and 120 hours.

In this test, the following compounds at 500 ppm showed a mortality of at least 75% in comparison with untreated controls: C.2, C.4, C.5, C.6, C.7, C.10, C.11, C.12, C.15.

B.4 Silverleaf Whitefly (bemisia argentifolii)

The active compounds were formulated in cyclohexanone as a 10,000 ppm solution supplied in tubes. The tubes were inserted into an automated electrostatic sprayer equipped with an atomizing nozzle and they serve as stock solutions for which lower dilutions are made in 50% acetone:50% water (v/v). A nonionic surfactant (Kinetic®) was included in the solution at a volume of 0.01% (v/v).

Cotton plants at the cotyledon stage (one plant per pot) were sprayed by an automated electrostatic plant sprayer equipped with an atomizing spray nozzle. The plants were dried in the sprayer fume hood and then removed from the sprayer. Each pot was placed into a plastic cup and about 10 to 12 whitefly adults (approximately 3-5 days old) were introduced. The insects were collected using an aspirator and a nontoxic Tygon® tubing connected to a barrier pipette tip. The tip, containing the collected insects, were then gently inserted into the soil containing the treated plant, allowing insects to crawl out of the tip to reach the foliage for feeding. Cups were covered with a reusable screened lid. Test plants were maintained in a growth room at about 25° C. and about 20-40% relative humidity for 3 days, avoiding direct exposure to fluorescent light (24 hour photoperiod) to prevent trapping of heat inside the cup. Mortality was assessed 3 days after treatment, compared to untreated control plants.

In this test, the following compounds at 500 ppm showed a mortality of at least 75% in comparison with untreated controls: C.4, C.10, C.11, C.12, C.13, C.15.

1-24. (canceled)

25. A method for combating or controlling invertebrate pests or for protecting crops, plants, plant propagation material and/or growing plants from attack or infestation by invertebrate pests comprising contacting the invertebrate pests, or their food supply, habitat or breeding grounds with a substituted 3-pyridyl thiazole compound of the general formula (I) or a composition comprising at least one compound of formula (I) 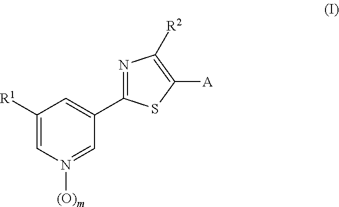
Wherein
m is 0 or 1;
R¹ is selected from the group consisting of hydrogen, cyano and halogen;
R² is selected from the group consisting of halogen and C₁-C₆-haloalkyl, the latter may be partially or fully halogenated and may optionally be further substituted by 1, 2, 3 or 4 radicals R⁷;
A is
a 4-, 5-, 6- or 7-membered heterocyclic or heteroaromatic ring, or
a 8-10-membered heteroaromatic bicyclic ring,
wherein each ring is on its carbon atoms optionally substituted with 1, 2, 3, 4, 5 or 6 substituents R⁶, wherein each
R⁶, when R² is C₁-C₆-haloalkyl and the number of R⁶ present is 0, 1, 2, 3, 4, 5 or 6, is selected independently from one another from the group consisting of hydrogen, halogen, cyano, azido, nitro, SCN, SF₅, C₁-C₁₀-alkyl, C₃-C₈-cycloalkyl, C₂-C₁₀-alkenyl, C₂-C₁₀-alkinyl, and wherein the carbon atoms of the aforementioned aliphatic and cycloaliphatic radicals may optionally be further substituted independently from one another with one or more R⁷, OR⁸, NR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, Si(R¹¹)₂R¹²;
phenyl, optionally substituted with 1, 2, 3, 4 or 5 substituents selected independently from R¹⁰;
and a 3-, 4-, 5-, 6- or 7-membered saturated, partly saturated or unsaturated aromatic heterocyclic ring comprising 1, 2, 3 or 4 heteroatoms selected from oxygen, nitrogen and/or sulfur, optionally substituted with 1, 2, 3, 4, or 5 substituents selected independently from R¹⁰, and wherein the nitrogen and/or the sulfur atom(s) of the heterocyclic ring may optionally be oxidized;
or two R⁶ present on one ring carbon atom may together form ═O, ═CR¹³R¹⁴; ═S; ═S(O)ₙR¹⁶; ═S(O)₆NR^{17a}R^{17b}, ═NR^{17a}, ═NOR¹⁶; ═NNR^{17a};
or two R⁶ together form a C₂-C₇ alkylene chain, thus forming, together with the ring atoms to which they are bound, a 3-, 4-, 5-, 6-, 7- or 8-membered ring, where the alkylene chain may be interrupted by 1 or 2 O, S or NR^{17a} or 1 or 2 of the CH₂ groups of the alkylene chain may be replaced by a group C═O, C═S or C═NR^{17a}; or the alkylene chain may be substituted by one or more radicals selected from the group consisting of halogen, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R¹⁸, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁸;
or,
R⁶, when R² is halogen and the number of R⁶ present is 1, 2, 3, 4, 5 or 6, is selected from the group consisting of halogen, cyano, azido, nitro, SCN, SF₅, C₃-C₈-cycloalkyl, C₂-Cₘ-alkenyl, C₂—Cm-alkinyl, and wherein the carbon atoms of the aforementioned aliphatic and cycloaliphatic radicals may optionally be further substituted independently from one another with one or more R⁷,
OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, and Si(R¹¹)₂R¹²;
or two R⁶ present on one ring carbon atom may together form ═O, ═CR¹³R¹⁴; ═S; —NR^{17a}; ═NOR¹⁶; ═NR^{17a};
or two R⁶ together form a C₂-C₇ alkylene chain, thus forming, together with the ring atoms to which they are bound, a 3-, 4-, 5-, 6-, 7- or 8-membered ring, where the alkylene chain may be interrupted by 1 or 2 O, S or NR^{17a} or 1 or 2 of the CH₂ groups of the alkylene chain may be replaced by a group C═O, C═S and/or C═NR^{17a}; or the alkylene chain may be substituted by one or more radicals selected from the group consisting of halogen, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R¹⁸, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁸;
or
R⁶, when R² is halogen and the number of R⁶ present is 2, 3, 4, 5 or 6, is selected from the group consisting of halogen, cyano, azido, nitro, SCN, SF₅, C₃-C₈-cycloalkyl, C₂-C₁₀-alkenyl, C₂-C₁₀-alkinyl, and wherein the carbon atoms of the aforementioned aliphatic and cycloaliphatic radicals may optionally be further substituted independently from one another with one or more R⁷,
OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, and Si(R¹¹)₂R¹²;
or two R⁶ present on one ring carbon atom may together form ═O, ═CR¹³R¹⁴; ═S; ═NR^{17a}, ═NOR¹⁶; ═NNR^{17a};
phenyl, optionally substituted with
1, 2, 3, 4, or 5 substituents selected independently from R¹⁰; or
a 3-, 4-, 5-, 6- or 7-membered saturated, partly saturated or unsaturated aromatic heterocyclic ring comprising 1, 2, 3 or 4 heteroatoms selected from the group consisting of oxygen, nitrogen and sulfur, optionally substituted with 1, 2, 3, 4, or 5 substituents selected independently from R¹⁰, and wherein the nitrogen or the sulfur atom(s) of the heterocyclic ring may optionally be oxidized
or two R⁶ together form a C₂-C₇ alkylene chain, thus forming, together with the ring atoms to which they are bound, a 3-, 4-, 5-, 6-, 7- or 8-membered ring, where the alkylene chain may be interrupted by 1 or 2 O, S or NR^{17a} or 1 or 2 of the CH₂ groups of the alkylene chain may be replaced by a group C═O, C═S or C═NR^{17a}; or the alkylene chain may be substituted by one or more radicals selected from the group consisting of halogen, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl,
phenyl, which may be substituted by 1, 2, 3, 4 or 5 radicals R¹⁸, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁸;
and wherein
R⁷ is each independently from one another selected from the group consisting of hydrogen, halogen, cyano, azido, nitro, —SCN, SF₅, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl, Si(R¹¹)₂R¹², OR¹⁶, OSO₂R¹⁶, S(O)ₙR¹⁶, S(O)ₙNR^{17a}R^{17b}, NR^{17a}R^{17b}, C(═O)NR^{17a}R^{17b}, C(═S)NR^{17a}R^{17b}, C(═O)OR¹⁶,
phenyl, optionally substituted with 1, 2, 3, 4 or 5 substituents R¹⁸, which are independently selected from one another, and
a 3-, 4-, 5-, 6- or 7-membered saturated, partly saturated or unsaturated aromatic heterocyclic ring comprising 1, 2 or 3 heteroatoms selected from the group consisting of oxygen, nitrogen and sulfur, optionally substituted with 1, 2, 3 or 4, substituents R¹⁸, selected independently from one another, and wherein the nitrogen and/or the sulfur atom(s) of the heterocyclic ring may optionally be oxidized,
or
two R⁷ present on one carbon atom may together form ═O, ═CR¹³R¹⁴; ═S; ═S(O)ₙR¹⁶; ═S(O)ₙNR^{17a}R^{17b}, ═NR^{17a}, ═NOR¹⁶; ═NNR^{17a};
or
two R⁷ may form a 3-, 4-, 5-, 6-, 7- or 8-membered saturated or partly unsaturated carbocyclic or heterocyclic ring together with the carbon atoms to which the two R⁷ are bonded;
R⁸ is each independently from one another selected from the group consisting of hydrogen, cyano, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₄-C₈-alkylcycloalkyl, C₃-C₈-halocycloalkyl, C₂-C₆-alkenyl, C₂-C₆-haloalkenyl, C₂-C₆-alkinyl, C₂-C₆ haloalkinyl, —Si(R¹¹)₂R¹², S(O)ₙR¹⁶, S(O)ₙNR^{17a}R^{17b}, NR^{17a}R^{17b}, —N═CR¹³R¹⁴, —C(═O)R¹⁵, C(═O)NR^{17a}R^{17b}, C(═S)NR^{17a}R^{17b}, C(═O)OR¹⁶,
phenyl, optionally substituted with one or more substituents R¹⁸, which are selected independently from one another, and
a 3-, 4-, 5-, 6- or 7-membered saturated, partly saturated or unsaturated aromatic heterocyclic ring comprising 1, 2 or 3 heteroatoms selected from the group consisting of oxygen, nitrogen and sulfur, optionally substituted with 1, 2, 3 or 4, substituents R¹⁸, selected independently from one another, and wherein the nitrogen and/or the sulfur atom(s) of the heterocyclic ring may optionally be oxidized;
R^{9a}, R^{9b} are each independently from one another selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl,
S(O)ₙR¹⁶, —S(O)ₙNR^{17a}R^{17b}, C(═O)R¹⁵, C(═O)OR¹⁶, C(═O)NR^{17a}R^{17b}, C(═S)R¹⁵, C(═S)SR¹⁶, C(═S)NR^{17a}R^{17b}, C(═NR^{17a})R¹⁵;
phenyl, optionally substituted with 1, 2, 3 or 4, substituents R¹⁸, which are selected independently from one another; and
a 3-, 4-, 5-, 6- or 7-membered saturated, partly saturated or unsaturated aromatic heterocyclic ring comprising 1, 2, 3 or 4 heteroatoms selected from the group consisting of oxygen, nitrogen and sulfur, optionally substituted with 1, 2, 3 or 4, substituents R¹⁸, selected independently from one another, and wherein the nitrogen or the sulfur atom(s) of the heterocyclic ring may optionally be oxidized;
or,
R^{9a} and R^{9b} are together a C₂-C₇ alkylene chain and form a 3-, 4-, 5-, 6-, 7- or 8-membered saturated, partly saturated or unsaturated aromatic ring together with the nitrogen atom to which they are bonded, wherein the alkylene chain may contain one or two heteroatoms selected from the group consisting of oxygen, sulfur and nitrogen, and may optionally be substituted with halogen, C₁-C₆-alkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆ haloalkinyl,
phenyl, optionally substituted with one or more substituents R¹⁸, which are selected independently from one another, or
a 3-, 4-, 5-, 6,- or 7-membered saturated, partly saturated or unsaturated aromatic heterocyclic ring comprising 1, 2 or 3 heteroatoms selected from the group consisting of oxygen, nitrogen and sulfur, optionally substituted with one or more substituents R¹⁸, selected independently from one another, and wherein the nitrogen or the sulfur atom(s) of the heterocyclic ring may optionally be oxidized;
or
R^{9a} and R^{9b} together may form a ═CR¹³R¹⁴, ═S(O)ₙ(R¹⁶)₂, ═NR¹⁷ or ═NOR¹⁶;
R¹⁰ is each independently from one another selected from the group consisting of hydrogen, halogen, cyano, azido, nitro, SCN, SF₅, C₁-C₁₀-alkyl, C₃-C₈-cycloalkyl, C₂-C₁₀-alkenyl, C₂-C₁₀-alkinyl, wherein the carbon atoms of the aforementioned aliphatic and cycloaliphatic radicals may optionally be substituted with one or more R¹⁵, which are selected independently from one another, Si(R¹¹)₂R¹², OR¹⁶, OS(O)ₙR¹⁶, —S(O)ₙR¹⁶, S(O)ₙNR^{17a}R^{17b}, NR^{17a}R^{17b}, C(═O)R¹⁵, C(═O)OR¹⁶, —C(═NR^{17a})R¹⁵, C(═O)NR^{17a}R^{17b}, C(═S)NR^{17a}R^{17b},
phenyl, optionally substituted with halogen, cyano, nitro, C₁-C₆-alkyl, C₁-C₆-haloalkyl, C₁-C₆-alkoxy or C₁-C₆-haloalkoxy, and
a 3-, 4-, 5-, 6- or 7-membered saturated, partly saturated or unsaturated aromatic heterocyclic ring comprising 1, 2 or 3 heteroatoms selected from the group consisting of oxygen, nitrogen and sulfur, optionally substituted with one or more substituents selected independently from one another from the group consisting of halogen, cyano, NO₂, C₁-C₆-alkyl, C₁-C₆-haloalkyl, C₁-C₆-alkoxy and C₁-C₆-haloalkoxy, and wherein the nitrogen or the sulfur atom(s) of the heterocyclic ring may optionally be oxidized;
or
two R¹⁰ present together on one atom of a partly saturated heterocyclic may be ═O, ═CR¹³R¹⁴; ═NR^{17a}, ═NOR¹⁶ or ═NNR^{17a};
or,
two R¹⁰ on adjacent carbon atoms may be a bridge selected from the group consisting of CH₂CH₂CH₂CH₂, CH═CH—CH═CH, N═CH—CH═CH, CH═N—CH═CH, N═CH—N═CH, OCH₂CH₂CH₂, OCH═CHCH₂, CH₂OCH₂CH₂, OCH₂CH₂O, OCH₂OCH₂, CH₂CH₂CH₂, CH═CHCH₂, CH₂CH₂O, CH═CHO, CH₂OCH₂, CH₂C(═O)O, C(═O)OCH₂, O(CH₂)O, SCH₂CH₂CH₂, SCH═CHCH₂, CH₂SCH₂CH₂, SCH₂CH₂S, SCH₂SCH₂, CH₂CH₂S, CH═CHS, CH₂SCH₂, CH₂C(═S)S, C(═S)SCH₂, S(CH₂)S, CH₂CH₂NR^{17a}, CH₂CH═N, CH═CH—NR^{17a}, OCH═N, and SCH═N and form together with the carbon atoms to which the two R¹⁰ are bonded to a 5-membered or 6-membered partly saturated or unsaturated, aromatic carbocyclic or heterocyclic ring, wherein the ring may optionally be substituted with one or two substituents selected from the group consisting of ═O, OH, CH₃, OCH₃, halogen, cyano, halomethyl and halomethoxy;
R¹¹, R¹² are each independently from one another selected from the group consisting of hydrogen, halogen, C₁-C₆ alkyl, C₁-C₆ haloalkyl, C₁-C₆ alkoxy, C₁-C₆ alkoxyalkyl, C₂-C₆ alkenyl, C₂-C₆ haloalkenyl, C₂-C₆ alkinyl, C₂-C₆ haloalkinyl, C₃-C₈ cycloalkyl, C₃-C₈ halocycloalkyl, C₁-C₆ alkoxyalkyl, C₁-C₆ haloalkoxyalkyl and phenyl, optionally substituted with one or more substituents R¹⁸, which are selected independently from one another;
R¹³, R¹⁴ are each independently from one another selected from the group consisting of hydrogen, C₁-C₄ alkyl, C₁-C₆ cycloalkyl, C₁-C₄ alkoxyalkyl, phenyl and benzyl;
R¹⁵ is each independently from one another selected from the group consisting of hydrogen, halogen, cyano, nitro, OH, SH, SCN, SF₅, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl, C₁-C₆-alkyl, C₂-C₆-alkenyl, C₂-C₆-alkinyl, C₃-C₈-cycloalkyl, wherein the four last mentioned aliphatic and cycloaliphatic radicals may be unsubstituted, partially or fully halogenated or oxgenated or may carry 1 or 2 radicals selected from C₁-C₄ alkoxy; phenyl, benzyl, pyridyl, and phenoxy, wherein the last four radicals may be unsubstituted, partially or fully halogenated or to carry 1, 2 or 3 substituents selected from the group consisting of C₁-C₆-alkyl, C₁-C₆-haloalkyl, C₁-C₆-alkoxy, C₁-C₆ haloalkoxy, (C₁-C₆-alkoxy)carbonyl, (C₁-C₆-alkyl)amino and di-(C₁-C₆-alkyl)amino,
or
two R¹⁵ present on the same carbon atom may together be ═O, ═CH(C₁-C₄), ═C(C₁-C₄-alkyl), ═N(C₁-C₆-alkyl) or ═NO(C₁-C₆-alkyl);
R¹⁶ is each independently from one another selected from the group consisting of hydrogen, cyano, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl, C₁-C₆-alkyl, C₂-C₆-alkenyl, C₂-C₆-alkinyl, and C₃-C₈-cycloalkyl, wherein the four last mentioned radicals may be unsubstituted, partially or fully halogenated or oxygenated or may carry 1 or 2 radicals selected from C₁-C₄ alkoxy; phenyl, benzyl, pyridyl, and phenoxy, wherein the last four radicals may be unsubstituted, partially or fully halogenated or carry 1, 2 or 3 substituents selected from the group consisting of C₁-C₆-alkyl, C₁-C₆-haloalkyl, C₁-C₆-alkoxy, C₁-C₆ haloalkoxy and (C₁-C₆-alkoxy)carbonyl;
R^{17a}, R^{17b} are each independently from one another selected from the group consisting of hydrogen, cyano, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl, C₁-C₆-alkyl, C₂-C₆-alkenyl, C₂-C₆-alkinyl, C₃-C₈-cycloalkyl, wherein the four last mentioned aliphatic and cycloaliphatic radicals may be unsubstituted, partially or fully halogenated and/or oxygenated and/or may carry 1 or 2 radicals selected from the group consisting of C₁-C₄-alkoxy;
phenyl, benzyl, pyridyl, and phenoxy, wherein the four last mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy or (C₁-C₆-alkoxy)carbonyl,
or,
R^{17a} and R^{17b} may together be a C₂-C₆ alkylene chain forming a 3- to 7-membered saturated, partly saturated or unsaturated ring together with the nitrogen atom R^{17a} and R^{17b} are bonded to, wherein the alkylene chain may contain 1 or 2 heteroatoms selected from the group consisting of oxygen, sulfur and nitrogen, and may optionally be substituted with halogen, C₁-C₄-haloalkyl, C₁-C₄-alkoxy or C₁-C₄-haloalkoxy, and wherein the nitrogen or the sulfur atom(s) of the heterocyclic ring may optionally be oxidized;
R¹⁸ is each independently from one another selected from the group consisting of hydrogen, halogen, nitro, cyano, OH, SH, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl,
C₁-C₆-alkyl, C₂-C₆-alkenyl, C₂-C₆-alkinyl, C₃-C₈-cycloalkyl, wherein the four last mentioned aliphatic and cycloaliphatic radicals may be unsubstituted, partially or fully halogenated and/or oxygenated and/or may carry 1 or 2 radicals selected from C₁-C₄-alkoxy; phenyl, benzyl, pyridyl, and phenoxy, wherein the four last mentioned radicals may be unsubstituted, partially or fully halogenated and/or carry 1, 2 or 3 substituents selected the group consisting of from C₁-C₆-alkyl, C₁-C₆-haloalkyl, C₁-C₆-alkoxy, C₁-C₆ haloalkoxy), and (C₁-C₆-alkoxy)carbonyl;
or
two R¹⁸ present together on one atom of a partly saturated atom may be ═O, ═S, ═N(C₁-C₆-alkyl), ═NO(C₁-C₆-alkyl), ═CH(C₁-C₄alkyl) or ═C(C₁-C₄-alkyl)C₁-C₄-alkyl;
or,
two R¹⁸ on two adjacent carbon atoms may be together a C₂-C₆ alkylene chain, which form together with the carbon atom they are bonded to a 3-, 4-, 5-, 6- or 7-membered saturated, partly saturated or unsaturated aromatic, wherein the alkylene chain may contain 1 or 2 heteroatoms selected from the group consisting of oxygen, sulfur and nitrogen, and may optionally be substituted with halogen, C₁-C₄-haloalkyl, C₁-C₄-alkoxy or C₁-C₄-haloalkoxy, and wherein the nitrogen and/or the sulfur atom(s) of the heterocyclic ring may optionally be oxidized;
n is 0, 1 or 2;
or an enantiomer, diastereomer or agriculturally or veterinarily acceptable salt thereof.

26. A method for protecting crops, plants, plant propagation material and/or growing plants from attack or infestation by invertebrate pests comprising contacting or treating the crops, plants, plant propagation material and growing plants, or soil, material, surface, space, area or water in which the crops, plants, plant propagation material is stored or the plant is growing, with the substituted 3-pyridyl thiazole compound of claim 25.

27. A method for treating, controlling, preventing or protecting animals against infestation or infection by parasites by administering or applying orally, topically or parenterally to the animals the substituted 3-pyridyl thiazole compound of claim 25.

28. The method of claim 25, wherein
R¹ is selected from the group consisting of hydrogen and fluoro.

29. The method of claim 25, wherein
R² is a partially or fully halogenated C₁-C₄ haloalkyl.

30. The method of claim 25, wherein
R¹ is selected from the group consisting of hydrogen and fluoro;
R² is selected from the group consisting of CHF₂, CF₃, CHCl₂, CCl₃ and C₂-C₄ haloalkyl.

31. The method of claim 25, wherein the substituted 3-pyridyl thiazole compounds are of the general formula (I-3) 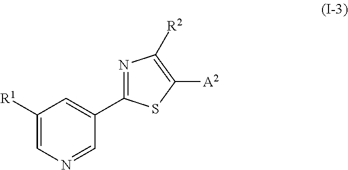
Wherein
R¹ is selected from the group consisting of hydrogen and fluoro;
R² is a partially or fully halogenated C₁-C₄ haloalkyl;
A² is a nitrogen containing 4-, 5-, or 6-membered heterocyclic or hetero-aromatic ring system, optionally substituted with 1, 2, 3, 4, 5, or 6 substituents R⁶, which are selected independently from one another;
R⁶ is selected from the group consisting of hydrogen, halogen, cyano, nitro, SCN, C₁-C₆-alkyl, C₃-C₆-cycloalkyl, C₂-C₆-alkenyl, C₂-C₆-alkinyl, wherein the aforementioned aliphatic and cycloaliphatic radicals may optionally be substituted with one or more R⁷, which are selected independently from one another,
OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b};
phenyl, optionally substituted with 1, 2, 3, 4, or 5 substituents selected independently from R¹⁰;
and a 3-, 4-, 5-, 6- or 7-membered saturated, partly saturated or unsaturated aromatic heterocyclic ring comprising 1, 2, 3 or 4 heteroatoms selected from oxygen, nitrogen and/or sulfur, optionally substituted with 1, 2, 3, 4, or 5 substituents selected independently from R¹⁰, and wherein the nitrogen and/or the sulfur atom(s) of the heterocyclic ring may optionally be oxidized;
or
two R⁶ present on one ring carbon atom may together form ═O, ═S, ═NR^{17a};

32. The method of claim 25, wherein
A is a nitrogen containing 4-, 5-, or 6-membered heterocyclic or hetero-aromatic ring system selected from any of the following ring systems D-1-D-135: 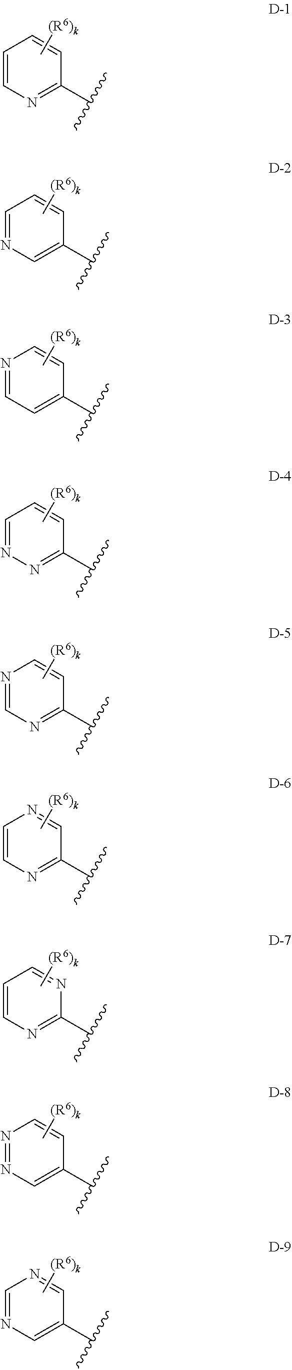 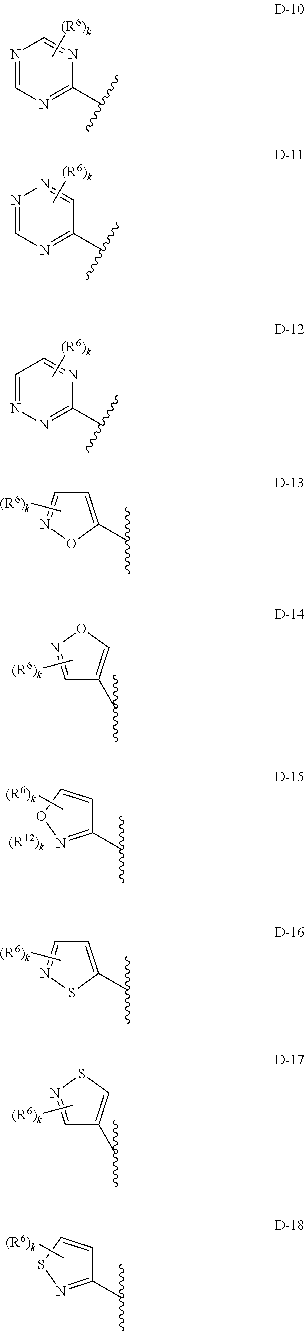 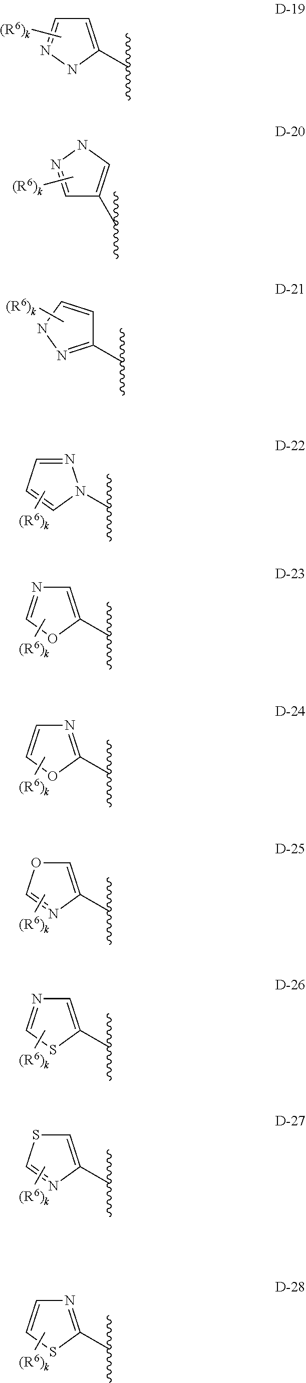 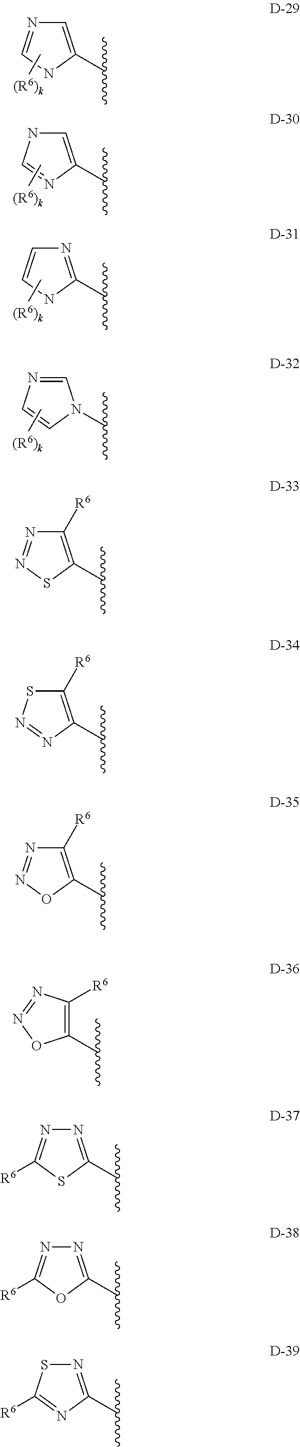 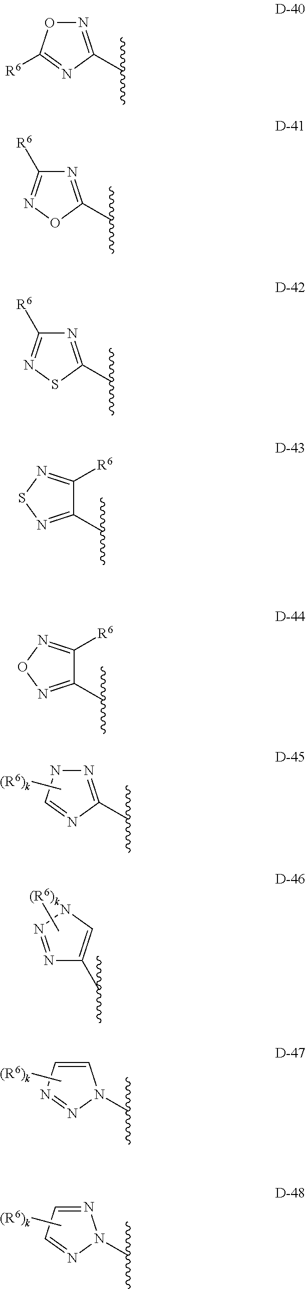 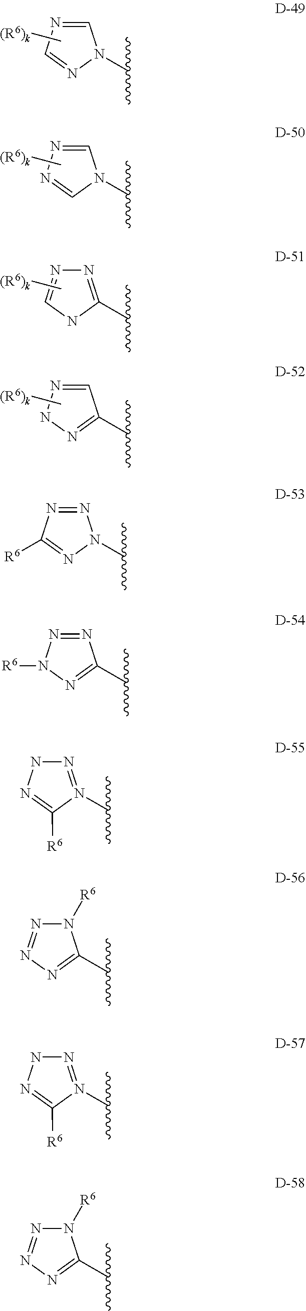 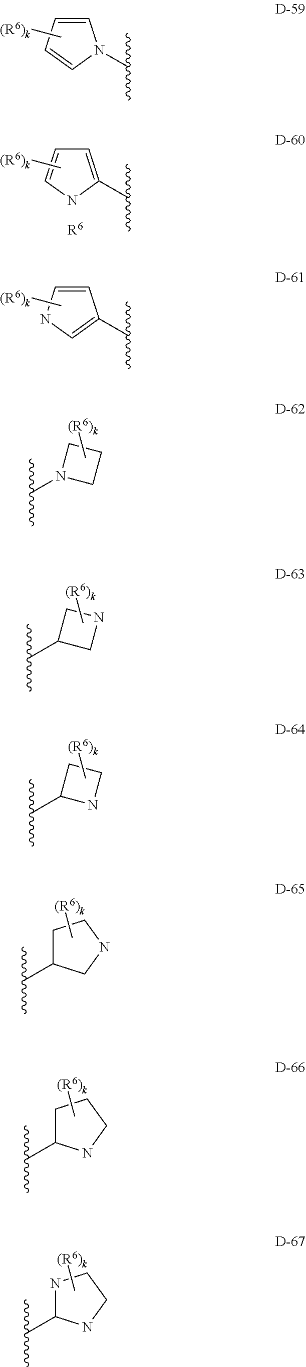 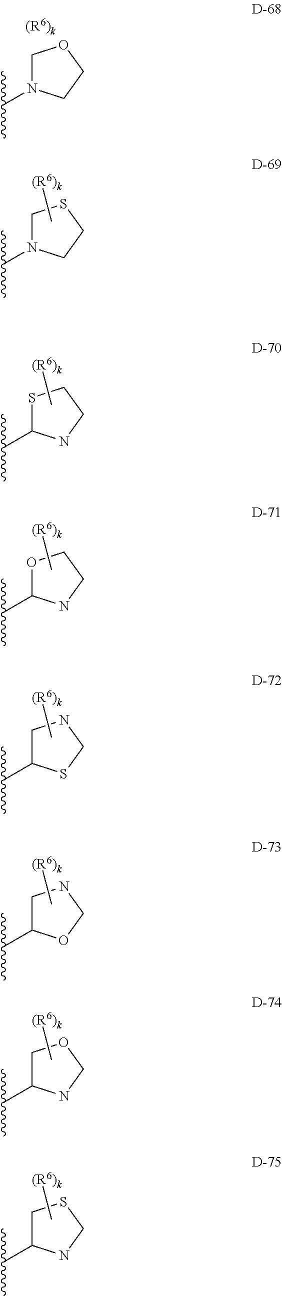 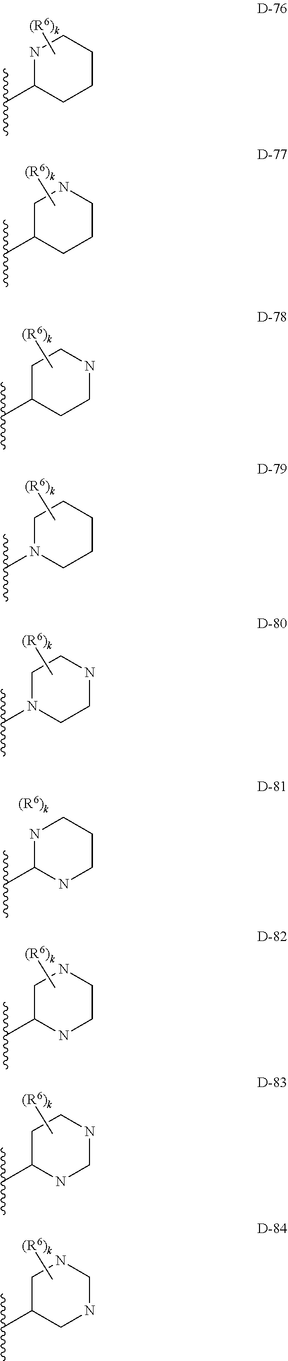 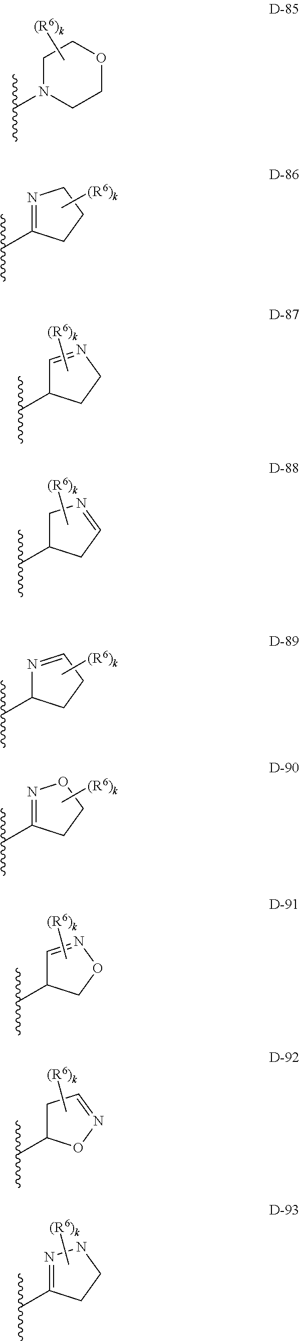 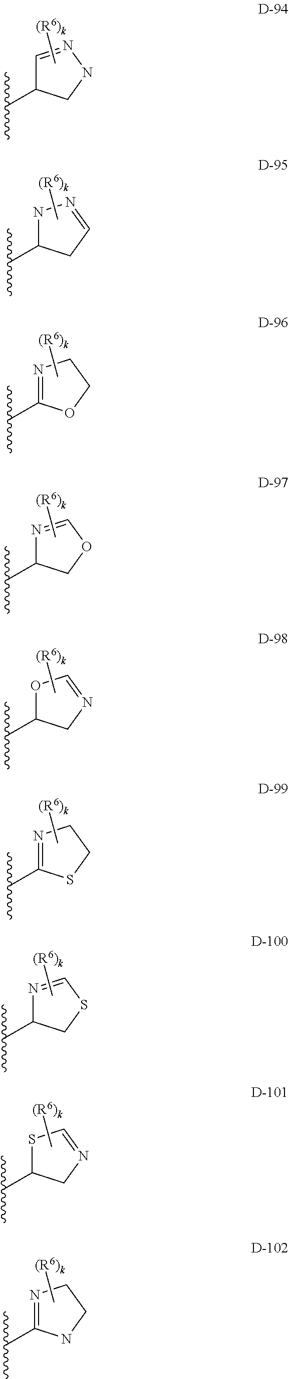 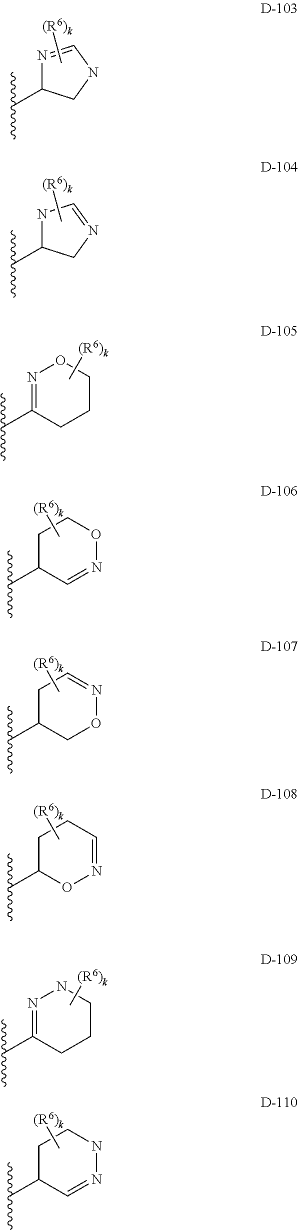 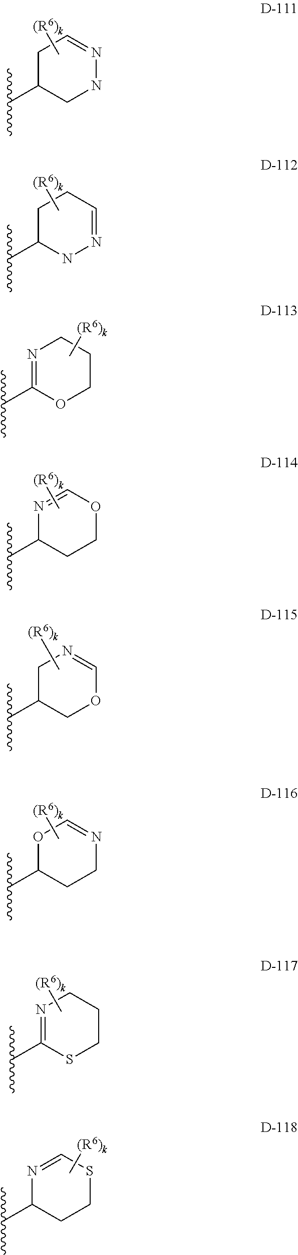 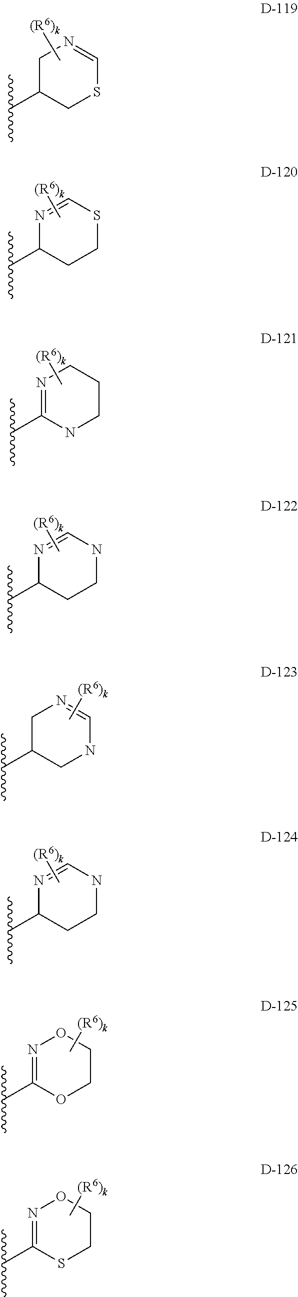 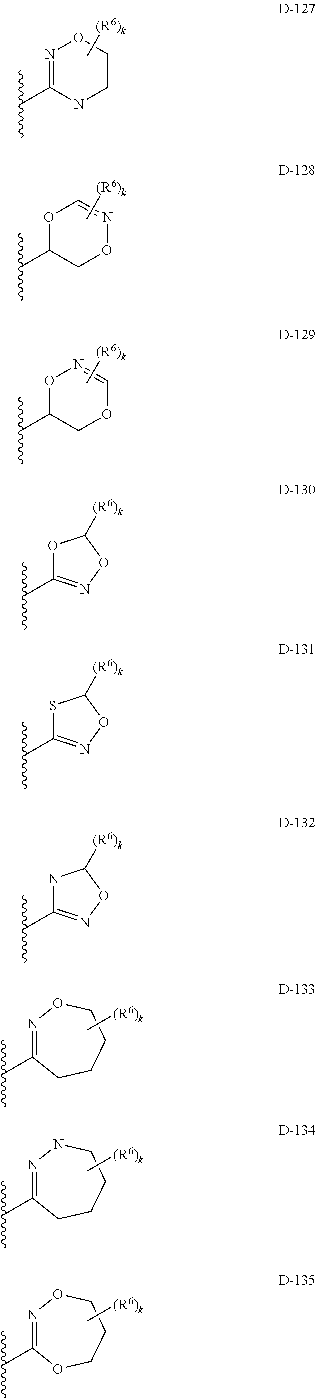
wherein the zigzag line denotes the bond to the thiazole ring of formula (I), k is an integer selected from 0, 1, 2, 3, 4, 5 or 6.

33. The method of claim 25, wherein
R² is a partially or fully halogenated C₁-C₄ haloalkyl;
and
A is a nitrogen containing 4-, 5-, or 6-membered heterocyclic or hetero-aromatic ring system selected from any of the following ring systems:
D-1, D-2, D-3, D-5, D-7, D-9, D-19, D-21, D-22, D-26, D-27, D-28, D-29, D-30, D-31, D-32, D-37, D-45, D-49, D-50, D-51, D-90, D-93, D-96, D-99, D-102, D-113, D-117, D-121, D-125, D-126, D-127, D-130, D-131, D-132, D-135, and wherein each
R⁶ is independently from one another selected from the group consisting of hydrogen, halogen, cyano, C₃-C₆-cycloalkyl, C₂-C₆-alkenyl, C₂-C6-alkinyl, and wherein the carbon atoms of the aforementioned aliphatic and cycloaliphatic radicals may optionally be further substituted independently from one another with one or more R¹⁵,
OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶, phenyl, pyridyl or pyrimidyl, each optionally substituted with 1, 2, 3, 4, or 5 substituents selected independently from R¹⁸;
or two R⁶ present on one ring carbon atom may together form ═O, ═S; ═NR^{17a};
and wherein
k is 0, 1, 2 or 3;

34. The method of claim 32, wherein
R² is halogen;
and
A is a nitrogen containing 4-, 5-, or 6-membered heterocyclic or hetero-aromatic ring system as defined in claim 8 selected from any of the following ring systems:
D-1, D-2, D-3, D-5, D-7, D-9, D-19, D-21, D-22, D-26, D-27, D-28, D-29, D-30, D-31, D-32, D-37, D-45, D-49, D-50, D-51, D-90, D-93, D-96, D-99, D-102, D-113, D-117, D-121, D-125, D-126, D-127, D-130, D-131, D-132, D-135, and wherein each
R⁶ is independently from one another selected from the group consisting of, halogen, cyano, C₁-C₁₆-alkyl, C₃-C₆-cycloalkyl, C₂-C₆-alkenyl, C₂-C6-alkinyl, and wherein the carbon atoms of the aforementioned aliphatic and cycloaliphatic radicals may optionally be further substituted independently from one another with one or more R¹⁵,
OR¹⁶, NR^{17a}R^{17b}, S(O)ₙR¹⁶, S(O)ₙNR^{17a}R^{17b}, C(═O)R¹⁵, C(═O)NR^{17a}R^{17b}, and C(═O)OR¹⁶;
or two R⁶ present on one ring carbon atom may together form ═O, ═CR¹³R¹⁴; ═S; ═NR^{17a}, ═NOR¹⁶; ═NNR^{17a};
and wherein
k is 1, 2 or 3;

35. The method of claim 32, wherein
R² is halogen;
and
A is a nitrogen containing 4-, 5-, or 6-membered heterocyclic or hetero-aromatic ring system as defined in claim 8 selected from any of the following ring systems:
D-1, D-2, D-3, D-5, D-7, D-9, D-19, D-21, D-22, D-26, D-27, D-28, D-29, D-30, D-31, D-32, D-37, D-45, D-49, D-50, D-51, D-90, D-93, D-96, D-99, D-102, D-113, D-117, D-121, D-125, D-126, D-127, D-130, D-131, D-132, D-135, and wherein each
R⁶ is independently from one another selected from the group consisting of, halogen, cyano, C₃-C₆-cycloalkyl, C₂-C₆-alkenyl, C₂-C₆-alkinyl, and wherein the carbon atoms of the aforementioned aliphatic and cycloaliphatic radicals may optionally be further substituted independently from one another with one or more R¹⁵,
OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶;
phenyl, optionally substituted with 1, 2, or 3, substituents selected independently from R¹⁰; and a 5- or 6-membered saturated, partly saturated or unsaturated aromatic heterocyclic ring comprising 1, 2, or 3 heteroatoms selected from oxygen, nitrogen and/or sulfur, optionally substituted with 1, 2, or 3 substituents selected independently from R¹⁰, and wherein the nitrogen and/or the sulfur atom(s) of the heterocyclic ring may optionally be oxidized, or two R⁶ present on one ring carbon atom may together form ═O, ═CR¹³R¹⁴; ═S; ═NR^{17a}, ═NOR¹⁶; ═NNR^{17a};
and wherein
k is 2 or 3;

36. A substituted 3-pyridyl thiazole compound of the general formula (I) 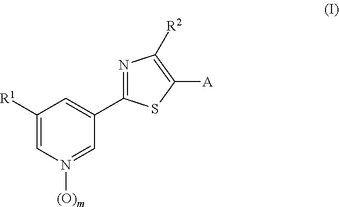
Wherein
m is 0 or 1;
R¹ is selected from the group consisting of hydrogen, cyano and halogen;
R² is selected from the group consisting of halogen and C₁-C₆-haloalkyl, the latter may be partially or fully halogenated and may optionally be further substituted by 1, 2, 3 or 4 radicals R⁷;
A is a molecular group representing a nitrogen containing ring system selected from
a 4-, 5-, 6- or 7-membered heterocyclic or heteroaromatic ring system, or
a 8-10-membered heteroaromatic bicyclic ring system,
wherein each ring is on its carbon atoms optionally substituted with one or more substituents R⁶, selected depending on the number of R⁶ present on the ring system and selected depending on the nature of R², wherein each
R⁶, when R² is C₁-C₆-haloalkyl and the number of R⁶ present is 0, 1, 2, 3, 4, 5 or 6, is selected independently from one another from the group consisting of hydrogen, halogen, cyano, azido, nitro, SCN, SF₅, C₃-C₈-cycloalkyl, C₂-C₁₀-alkenyl, C₂-C₁₀-alkinyl, and wherein the carbon atoms of the aforementioned aliphatic and cycloaliphatic radicals may optionally be further substituted independently from one another with one or more R⁷, OR⁸, NR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, Si(R¹¹)₂R¹²;
phenyl, optionally substituted with 1, 2, 3, 4 or 5 substituents selected independently from R¹⁰;
a 3-, 4-, 5-, 6- or 7-membered saturated, partly saturated or unsaturated aromatic heterocyclic ring comprising 1, 2, 3 or 4 heteroatoms selected from the group consisting of oxygen, nitrogen and sulfur, optionally substituted with 1, 2, 3, 4, or 5 substituents selected independently from R¹⁰, and wherein the nitrogen and/or the sulfur atom(s) of the heterocyclic ring may optionally be oxidized;
or two R⁶ present on one ring carbon atom may together form ═O, ═CR¹³R¹⁴; ═S; ═S(O)ₙR¹⁶; ═S(O)ₙNR^{17a}R^{17b}, ═NR^{17a}, ═NOR¹⁶; ═NNR^{17a};
or two R⁶ together form a C₂-C₇ alkylene chain, thus forming, together with the ring atoms to which they are bound, a 3-, 4-, 5-, 6-, 7- or 8-membered ring, where the alkylene chain may be interrupted by 1 or 2 O, S and/or NR^{17a} and/or 1 or 2 of the CH₂ groups of the alkylene chain may be replaced by a group C═O, C═S and/or C═NR^{17a}; and/or the alkylene chain may be substituted by one or more radicals selected from the group consisting of halogen, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R¹⁸, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁸;
or,
R⁶, when R² is halogen and the number of R⁶ present is 1, 2, 3, 4, 5 or 6, is selected from the group consisting of halogen, cyano, azido, nitro, SCN, SF₅, C₃-C₈-cycloalkyl, C₂-C₁₀-alkenyl, C₂-C₁₀-alkinyl, and wherein the carbon atoms of the aforementioned aliphatic and cycloaliphatic radicals may optionally be further substituted independently from one another with one or more R⁷,
OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, Si(R¹¹)₂R¹²;
or two R⁶ present on one ring carbon atom may together form ═O, ═CR¹³R¹⁴; ═S; ═NR^{17a}, ═NOR¹⁶; ═NNR^{17a};
or two R⁶ together form a C₂-C₇ alkylene chain, thus forming, together with the ring atoms to which they are bound, a 3-, 4-, 5-, 6-, 7- or 8-membered ring, where the alkylene chain may be interrupted by 1 or 2 O, S and/or NR^{17a} and/or 1 or 2 of the CH₂ groups of the alkylene chain may be replaced by a group C═O, C═S and/or C═NR^{17a}; and/or the alkylene chain may be substituted by one or more radicals selected from the group consisting of halogen, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R¹⁸, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁸;
or
R⁶, when R² is halogen and the number of R⁶ present is 2, 3, 4, 5 or 6, is selected from the group consisting of halogen, cyano, azido, nitro, SCN, SF₅, C₁-C₁₀-alkyl, C₃-C₈-cycloalkyl, C₂-C₁₀-alkenyl, C₂-C₁₀-alkinyl, and wherein the carbon atoms of the aforementioned aliphatic and cycloaliphatic radicals may optionally be further substituted independently from one another with one or more R⁷,
OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, Si(R¹¹)₂R¹²;
or two R⁶ present on one ring carbon atom may together form ═O, ═CR¹³R¹⁴; ═S; ═NR^{17a}, ═NOR¹⁶; ═NNR^{17a};
phenyl, optionally substituted with 1, 2, 3, 4, or 5 substituents selected independently from R¹⁰; or
a 3-, 4-, 5-, 6- or 7-membered saturated, partly saturated or unsaturated aromatic heterocyclic ring comprising 1, 2, 3 or 4 heteroatoms selected from the group consisting of oxygen, nitrogen and sulfur, optionally substituted with 1, 2, 3, 4, or 5 substituents selected independently from R¹⁰, and wherein the nitrogen and/or the sulfur atom(s) of the heterocyclic ring may optionally be oxidized
or two R⁶ together form a C₂-C₇ alkylene chain, thus forming, together with the ring atoms to which they are bound, a 3-, 4-, 5-, 6-, 7- or 8-membered ring, where the alkylene chain may be interrupted by 1 or 2 O, S and/or NR^{17a} and/or 1 or 2 of the CH₂ groups of the alkylene chain may be replaced by a group C═O, C═S and/or C═NR^{17a}; and/or the alkylene chain may be substituted by one or more radicals selected from the group consisting of halogen, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl, which may be substituted by 1, 2, 3, 4 or 5 radicals R¹⁸, and
a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁸;
and wherein the other substituents
n, R⁷, R⁸, R^{9a}, R^{9a}, R¹⁰, R¹¹, R¹², R¹³, R¹⁴, R¹⁵, R¹⁶, R^{17a}, R^{17b} and R¹⁸ are defined as in claim 25;
and/or an enantiomer, diastereomer or agriculturally or veterinarily acceptable salts thereof.

37. The compound of claim 36, wherein
R¹ is selected from the groups consisting of hydrogen and fluoro.

38. The compound of claim 36, wherein
A is a nitrogen containing 4-, 5-, or 6-membered heterocyclic or hetero-aromatic ring system selected from any of the ring systems D-1-D-135.

39. The compound of claim 36, wherein
R² is partially or fully halogenated haloalkyl; and
A is a nitrogen containing 4-, 5-, or 6-membered heterocyclic or hetero-aromatic ring system, selected from any of the following ring systems:
D-1, D-2, D-3, D-5, D-7, D-9, D-19, D-21, D-22, D-26, D-27, D-28, D-29, D-30, D-31, D-32, D-37, D-45, D-49, D-50, D-51, D-90, D-93, D-96, D-99, D-102, D-113, D-117, D-121, D-125, D-126, D-127, D-130, D-131, D-132, D-135, and wherein each
R⁶ is selected independently from one another from the group consisting of hydrogen, halogen, cyano, azido, nitro, SCN, SF₅, C₁-C₁₀-alkyl, C₃-C₈-cycloalkyl, C₂-C₁₀-alkenyl, C₂-C₁₀-alkinyl, and wherein the carbon atoms of the aforementioned aliphatic and cycloaliphatic radicals may optionally be further substituted independently from one another with one or more R⁷, OR⁸, NR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, Si(R¹¹)₂R¹²;
phenyl, optionally substituted with 1, 2, 3, 4 or 5 substituents selected independently from R¹⁰;
and a 3-, 4-, 5-, 6- or 7-membered saturated, partly saturated or unsaturated aromatic heterocyclic ring comprising 1, 2, 3 or 4 heteroatoms selected from oxygen, nitrogen and sulfur, optionally substituted with 1, 2, 3, 4, or 5 substituents selected independently from R¹⁰, and wherein the nitrogen and/or the sulfur atom(s) of the heterocyclic ring may optionally be oxidized;
or two R⁶ present on one ring carbon atom may together form ═O, ═CR¹³R¹⁴; ═S; ═S(O)ₙR¹⁶; ═S(O)ₙNR^{17a}R^{17b}, ═NR^{17a}, ═NOR¹⁶; ═NNR^{17a};
or two R⁶ together form a C₂-C₇ alkylene chain, thus forming, together with the ring atoms to which they are bound, a 3-, 4-, 5-, 6-, 7- or 8-membered ring, where the alkylene chain may be interrupted by 1 or 2 O, S and/or NR^{17a} or 1 or 2 of the CH₂ groups of the alkylene chain may be replaced by a group C═O, C═S or C═NR^{17a}; or the alkylene chain may be substituted by one or more radicals selected from the group consisting of halogen, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R¹⁸, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁸;
and wherein
k is 0, 1, 2 or 3.

40. The compound of claim 36, wherein
R² is halogen; and
A is a nitrogen containing 4-, 5-, or 6-membered heterocyclic or hetero-aromatic ring system selected from any of the following ring systems:
D-1, D-2, D-3, D-5, D-7, D-9, D-19, D-21, D-22, D-26, D-27, D-28, D-29, D-30, D-31, D-32, D-37, D-45, D-49, D-50, D-51, D-90, D-93, D-96, D-99, D-102, D-113, D-117, D-121, D-125, D-126, D-127, D-130, D-131, D-132, D-135, and wherein each
R⁶ is selected from the group consisting of halogen, cyano, azido, nitro, SCN, SF₅, C₁-C₁₀-alkyl, C₃-C₈-cycloalkyl, C₂-C₁₀-alkenyl, C₂-C₁₀-alkinyl, and wherein the carbon atoms of the aforementioned aliphatic and cycloaliphatic radicals may optionally be further substituted independently from one another with one or more R⁷,
OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, and Si(R¹¹)₂R¹²;
or two R⁶ present on one ring carbon atom may together form ═O, ═CR¹³R¹⁴; ═S; ═NR^{17a}, ═NOR¹⁶; ═NNR^{17a};
or two R⁶ together form a C₂-C₇ alkylene chain, thus forming, together with the ring atoms to which they are bound, a 3-, 4-, 5-, 6-, 7- or 8-membered ring, where the alkylene chain may be interrupted by 1 or 2 O, S or NR^{17a} or 1 or 2 of the CH₂ groups of the alkylene chain may be replaced by a group C═O, C═S or C═NR^{17a}; or the alkylene chain may be substituted by one or more radicals selected from the group consisting of halogen, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R¹⁸, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁸;
and wherein
k is 1, 2 or 3.

41. The compound of claim 36, wherein
R² is halogen; and
A is a nitrogen containing 4-, 5-, or 6-membered heterocyclic or hetero-aromatic ring system selected from any of the following ring systems:
D-1, D-2, D-3, D-5, D-7, D-9, D-19, D-21, D-22, D-26, D-27, D-28, D-29, D-30, D-31, D-32, D-37, D-45, D-49, D-50, D-51, D-90, D-93, D-96, D-99, D-102, D-113, D-117, D-121, D-125, D-126, D-127, D-130, D-131, D-132, D-135, and wherein each
R⁶ is selected from the group consisting of halogen, cyano, azido, nitro, SCN, SF₅, C₁-C₁₀-alkyl, C₃-C₈-cycloalkyl, C₂-C₁₀-alkenyl, C₂-C₁₀-alkinyl, and wherein the carbon atoms of the aforementioned aliphatic and cycloaliphatic radicals may optionally be further substituted independently from one another with one or more R⁷,
OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, Si(R¹¹)₂R¹²;
or two R⁶ present on one ring carbon atom may together form ═O, ═CR¹³R¹⁴; ═NR^{17a}, ═NOR¹⁶; ═NNR^{17a};
phenyl, optionally substituted with 1, 2, 3, 4, or 5 substituents selected independently from R¹⁰;
and a 3-, 4-, 5-, 6- or 7-membered saturated, partly saturated or unsaturated aromatic heterocyclic ring comprising 1, 2, 3 or 4 heteroatoms selected from oxygen, nitrogen and sulfur, optionally substituted with 1, 2, 3, 4, or 5 substituents selected independently from R¹⁰, and wherein the nitrogen or the sulfur atom(s) of the heterocyclic ring may optionally be oxidized;
or two R⁶ together form a C₂-C₇ alkylene chain, thus forming, together with the ring atoms to which they are bound, a 3-, 4-, 5-, 6-, 7- or 8-membered ring, where the alkylene chain may be interrupted by 1 or 2 O, S or NR^{17a} or 1 or 2 of the CH₂ groups of the alkylene chain may be replaced by a group C═O, C═S or C═NR^{17a}; or the alkylene chain may be substituted by one or more radicals selected from the group consisting of halogen, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl, which may be substituted by 1, 2, 3, 4 or 5 radicals R¹⁸, and
a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁸;
and wherein
k is 2 or 3.

42. An intermediate compound of the formula (I-4) 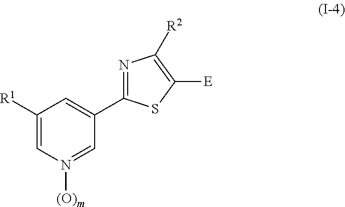
wherein
m is 0 or 1;
R¹ is selected from the group consisting of hydrogen and fluoro;
R² is selected from the group consisting of halogen and C₁-C₆-haloalkyl, the latter may be partially or fully halogenated and may optionally be further substituted by 1, 2, 3 or 4 radicals R⁷ as defined in claim 25;
E is selected from the group consisting of Cl, Br, I, the molecular group E1 and the molecular group E2, wherein
the molecular group E1 is 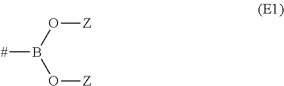
wherein # denotes the bond to the thiazole ring in formula (I-4), and each Z is independently from one another selected from the group consisting of hydrogen and C₁-C₄ alkyl; and
the molecular group (E2) is 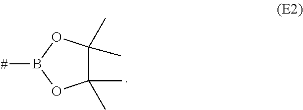
wherein # denotes the bond to the thiazole ring in formula (I-4).

43. An agricultural or veterinary composition comprising the compound of claim 25.

44. The method of claim 25, wherein the invertebrate pests or parasites are insects, arachnids or nematodes.

45. The method of claim 26, wherein the plant propagation material are seeds.

46. A seed treated with the compound of claim 25.
